(19) United States
(12) Patent Application Publication
(10) Pub. No.: US 2004/0019187 A1
(43) Pub. Date: Jan. 29, 2004

(54) Immunomodulatory human mhc class II antigens-binding polypeptides

(76) Inventors: Zoltan Nagy, Munchen (DE); Michael Tesar, Weilheim (DE); Elisabeth Thomassen-Wolf, Munchen (DE)

Correspondence Address: ROPES & GRAY LLP, ONE INTERNATIONAL PLACE, BOSTON, MA, 02110-2624, US

(21) Appl. No.: 10/275,046

(22) PCT Filed: May 14, 2001
(86) PCT No.: PCT/US01/15626

(30) Foreign Application Priority Data
May 12, 2000 (EP) 00110063.5

(51) Int. Cl. C07K 16/28

(52) U.S. Cl. 530/388.22

(57) ABSTRACT

The present invention relates to human polypeptides causing or leading to the modulation of the immune system. The invention further relates to nucleic acids encoding the polypeptides, methods for production of the polypeptides, methods for immunosuppression, pharmaceutical and diagnostic compositions and kits comprising the polypeptides and uses of the polypeptides.

### BACKGROUND OF THE INVENTION

Diseases involving the immune system are significantly debilitating to suffering individuals and are predicted to increase in prevalence over the next 10 years. Such diseases include rheumatoid arthritis (RA), multiple sclerosis (MS), type I diabetes, transplant rejection (TR) and graft vs. host disease (GvHD). For example, the number of patients suffering from rheumatoid arthritis is expected to grow world-wide from 6.6 million to 7 million by 2010. The recorded number of patients suffering from these diseases in 1995, the predicted number of patients for 2010 and corresponding market sizes are shown below.

| | |
|---|---|
| Number of | |
| patients (Mio) | Market size (Bio.$) |

| Disease | 1995 | 2010 (est) | 1994 | 2010 (est) |
|---|---|---|---|---|
| Rheumatoid Arthritis | 6.6 | 7 | 2.4 | >3.7 |
| Multiple Sclerosis | 0.62 | 0.65 | 0.3 | >1.5 |
| Type I diabetes | 1.8 | 1.9 | 1.5 | >1.5 |
| Transplant/GvHD | 0.05 | 0.1 | 0.9 | >1.5 |

Current therapies in diseases of the immune system include anti-inflammatory drugs, e.g., NSAIDS (non-steroidal anti-inflammatory drugs), corticosteroids, cytostatics (methotrexate for RA), and cytokines (interferon beta for MS). These therapies are symptomatic; none of them induces complete remission of the disease. A general problem with most current drugs is also their lack of selectivity: they suppress the whole immune system, and thus, the patients treated will become highly susceptible to infections. Finally, the side effect profile of most presently used anti-inflammatory agents also warrants the development of new therapeutics for these diseases. Therefore, there is a pressing unmet medical need for selective, disease-mechanism-based therapeutics to treat diseases of the immune system such as RA and MS.

The underlying immunological mechanisms of TR and GvHD are similar to those of other diseases of the immune system. In TR, the recipient's immune system attacks the foreign organ, whereas in GvHD the foreign hematopoietic cells introduced into immunocomprimised hosts attack the host. At present, corticosteroids, Azathioprine, Cydosporin A, and CellCept are used for prevention of rejection, and high dose corticosteroids, OKT3 (a monoclonal antibody (mAb) to a pan-T cell marker), and Zenapax (mAb to IL-2R on activated T cells) for its treatment. In GvHD corticosteroids are used, but no satisfactory treatment is available. There is an unmet medical need for a better tolerated and more effective immunosuppressant, particularly for the treatment of GvHD.

Every mammalian species, which has been studied to date carries a cluster of genes coding for the so called major histocompatibility complex (MHC). This tightly linked cluster of genes code for surface antigens, which play a central role in the development of both humoral and cell-mediated immune responses. In humans the products coded for by the MHC are referred to as Human Leukocyte Antigens or HLA. The MHC-genes are organised into regions encoding three classes of molecules, class I to III.

Class I MHC molecules are 45 kD transmembrane glycoproteins, noncovalently associated with another glycoprotein, the 12 kD beta-2 microglobulin (Brown et al., 1993). The latter is not inserted into the cell membrane, and is encoded outside the MHC. Human class I molecules are of three different isotypes, termed HLA-A, -B, and -C, encoded in separate loci. The tissue expression of class I molecules is ubiquitous and codominant. MHC class I molecules present peptide antigens necessary for the activation of cytotoxic T-cells.

Class II MHC molecules are noncovalently associated heterodimers of two transmembrane glycoproteins, the 35 kD α chain and the 28 kD β chain (Brown et al., 1993). In humans, class II molecules occur as three different isotypes, termed human leukocyte antigen DR (HLA-DR), HLA-DP, and HLA-DQ. Polymorphism in DR is restricted to the β chain, whereas both chains are polymorphic in the DP and DQ isotypes. Class II molecules are expressed codominantly, but in contrast to class I, exhibit a restricted tissue distribution: they are present only on the surface of cells of the immune system, for example dendritic cells, macrophages, B lymphocytes, and activated T lymphocytes. They are also expressed on human adrenocortical cells in the zona reticularis of normal adrenal glands and on granulosa-lutein cells in corpora lutea of normal ovaries (Kahoury et al., 1990). Their major biological role is to bind antigenic peptides and present them on the surface of antigen presenting cells (APC) for recognition by CD4 helper T (Th) lymphocytes (Babbitt et al., 1985.) MHC class II molecules can also be expressed on the surface of non-immune system cells. For example, cells in an organ other than lymphoid cells can express MHC class II molecules during a pathological inflammatory response. These cells may include synovial cells, endothelial cells, thyroid stromal cells and glial cells.

Class III MHC molecules are also associated with immune responses, but encode somewhat different products. These include a number of soluble serum proteins, enzymes and proteins like tumour necrosis factor or steroid 21-hydroxylase enzymes. In humans, class III molecules occur as three different isotypes, termed Ca, C2 and Bf (Kuby, 1994 the page number for this reference is mising).

A large body of evidence has demonstrated that susceptibility to many diseases, in particular diseases of the immune system, is strongly associated with specific alleles of the major histocompatibility complex (reviewed in Tiwari et al., 1985). Although some class I associated diseases exist, most autoimmune conditions have been found to be associated with class II alleles. For example, class II alleles DRB1*0101, 0401, 0404, and 0405 occur at increased frequency among rheumatoid arthritis (RA) patients (McMichael et al., 1977; Stasny, 1978; Ohta et al., 1982; Schiff et al., 1982), whereas DRB1*1501 is associated with multiple sclerosis (MS), and the DQ allele combination DQA1*0301/B1*0302 with insulin dependent diabetes mellitus;(IDDM). In RA, altogether >94% of rheumatoid factor positive patients carry one of the susceptibility alleles (Nepom et al., 1989).

Class II MHC molecules are the primary targets for immunosuppressive intervention for the following reasons: First, MHC-II molecules activate T helper (Th) cells that are central to immunoregulation, and are responsible for most of the immunopathology in inflammatory diseases. Second, most diseases of the immune system are genetically associated with class II alleles. Third, MHC-II molecules are only expressed on cells of the immune system, whereas MHC-I molecules are present on most somatic cells.

At least three mechanisms are believed to play some part in immunosuppression mediated by proteins binding to MHC class II molecules. First, since Th cells recognise antigenic peptides bound to class II molecules, monoclonal antibodies (mAb) specific for class II moleculescan sterically hinder the interaction between the MHC class II molecule and the T cell receptor, and thereby prevent Th cell activation. Indeed, this has been shown to occur both in vitro and in vivo (Baxevanis et al., 1980; Nepom et al., 1981; Rosenbaum et al., 1983). Second, down regulation of cell surface expression of MHC class II molecules has been shown to associate with immunosuppression using certain mouse anti-MHC class II antibodies (Vidovic et al., 1995). Third, killing of activated lymphoid cells occurs when certain anti-MHC class II antibodies bind to antigen expressed on the surface of these cells (Vidovic et al., 1995a). Increased selectivity of treatment is achieved since only cells expressing the specific MHC class II antigen can be targeted by a specific monoclonal antibody and hence only the immune response mediated by these allotypes is modulated. Host-defence immune reactions which are mediated by other MHC molecules are not targeted by the specific antibody and hence remain unmodulated and non-comprimised.

Based on these observations, anti-class II mAb have been envisaged for a number of years as therapeutic candidates for the immunosuppressive treatment of disorders of the immune system including transplant rejection. Indeed, this hypothesis has been supported by the beneficial effect of mouse-derived anti-class II mAbs in a series of animal disease models (Waldor et al., 1983; Jonker et al., 1988; Stevens et al., 1990; Smith et al., 1994).

Despite these early supporting data, to date no anti-MHC class II mAb of human composition has been described that displays the desired immunomodulatory and other biological properties that may include affinity, inhibition of proliferation or reduction in cytokine secretion. Indeed, despite the relative ease by which mouse-derived mAbs may be obtained, work using mouse-derived mAbs has demonstrated the difficulty of obtaining an immunomodulatory antibody with the desired biological properties. For example, significant and not fully understood differences were observed in the T cell inhibitory capacity of different murine anti-class II mAbs (Naquet et al., 1983). Furthermore, the application of certain mouse-derived mAbs in vivo was associated with unexpected side effects, sometimes resulting in death of laboratory primates (Billing et al., 1983; Jonker et al., 1991).

It is generally accepted that mouse-derived mAbs (including chimeric and so-called ‘humanized’ mAbs) carry an increased risk of generating an adverse immune response (Human anti-murine antibody—HAMA) in patients compared to treatment with a human mAb (for example, Vose et al, 200; Kashmiri et al., 2001). This risk is potentially increased when treating chronic diseases such as rheumatoid arthritis or multiple sclerosis with any mouse-derived mAb; prolonged exposure of the human immune system to a non-human molecule often leads to the development of an adverse immune reaction. Furthermore, it is has proven very difficult to obtain mouse-derived antibodies with the desired specificity or affinity to the desired antigen (Pichla et al. 1997). Such observations may have a significant influence or reduce the overall therapeutic effect or advantage provided by mouse-derived mAbs. Examples of disadvantages for mouse-derived mAbs may include the following. First, mouse-derived mAbs may be limited in the medical conditions or length of treatment for a condition for which they are appropriate. Second, the dose rate for mouse-derived mAbs may need to be relatively high in order to compensate for a relatively low affinity or therapeutic effect (low affinity or theraputic effect are not associated with murine origin. Half life in the human body, however, may be, as a murine mAb would likely be cleared more quickly from the blood. This could also necessitate higher dosing), hence making the dose not only more severe but potentially more immunogenic and perhaps dangerous. Third, such restrictions in suitable treatment regimes and high-dose rates that require high production amounts may significantly add to the cost of treatment and could mean that such a mouse-derived mAb be uneconomical to develop as a commercial therapeutic. Finally, even if a mouse mAb could be identified that displayed the desired specificity or affinity, often these desired features are detrimentally affected during the ‘humanization’ or ‘chimerization’ procedures necessary to reduce immunogenic potential (Slavin-Chiorini et al., 1997). Once a mouse-derived mAb has been ‘humanized’ or chimerized, then it is very difficult to optimize its specificity or affinity.

The art has sought over a number of years for anti-MHC class II mAbs of human composition that show immunomodulatory and other biological properties suitable for use in a pharmaceutical composition for the treatment of humans. Workers in the field have practised the process steps of first identifying a mouse-derived mAb, and then modifying the structure of this mAb with the aim of improving immunotolerance of this non-human molecule for human patients (for further details, see Jones et al., 1986, Riechmann et al., 1988; Presta, 1992). This modification is typically made using so-called ‘humanisation’ procedures or by fabricating a human-mouse chimeric mAb. Other workers have attempted to identify human antibodies that bind to human antigens having desired properties within natural repertoires of human antibody diversity. For example, by exploring the foetal-tolerance mechanism in pregnant women (Bonagura et al.,1987) or by panning libraries of natural diversifies of antibodies (Stausbøl-Grøn et al., 1996; Winter et al., 1994). However, to date no anti-MHC class II mAb of human composition has been described that displays the desired biological properties of immunomodulation, specificity, low immunogenicity and affinity.

### SUMMARY OF THE INVENTION

One aspect of the present invention provides a composition including a polypeptide comprising at least one antibody-based antigen-binding domain of human composition with a binding specificity for an antigen expressed on the surface of a cell. In preferred embodiments, treating cells (lymphoids or non-lymphoid cells) expressing the antigen with one or more of the polypeptides causes or leads to suppression of an immune response, e.g., wherein the IC50 for the suppressive activity is 1 μM or lower, and even more preferably 100 nM, 10 nM or even 1 nM or less.

In certain preferred embodiments, the antibody-based antigen-binding domain comprises a monovalent antibody fragment selected from Fv, scFv, dsFv and Fab fragment. In other preferred embodiments, the polypeptide comprises an F(ab)′₂ antibody fragment or a mini-antibody fragment. In a further preferred embodiment the polypeptide is a multivalent composition comprising at least one full antibody selected from IgG1, IgG2a, IgG2b, IgG3, IgG4, IgA and IgM.

According to a preferred embodiment, the polypeptide is directed to a lymphoid cell or a non-lymphoid cell that expresses MHC class II molecules. The latter type of cells occurs for example at pathological sites of inflammation and/or diseases of the immune system. Said cells may include synovial cells, endothelial cells, thyroid stromal cells and glial cells.

In certain embodiments, the polypeptide binds to at least one epitope in the alpha-chain of an HLA-DR molecule. According to a further preferred embodiment, the polypeptide binds to at least one epitope of the first domain of the alpha-chain of HLA-DR, e.g., the polypeptide binds to at least one epitope within the alpha-helix ranging from Glu⁵⁵ to Tyr⁷⁹ of the alpha-chain of HLA-DR.

In certain preferred embodiments, the polypeptide binds to at least one epitope in the beta-chain of an HLA-DR molecule, and preferably binds to at least one epitope of the first domain of the beta-chain of HLA-DR.

In certain embodiments, the subject polypeptide includes at least one antibody-based antigen-binding domain which specifically binds to a human MHC class II antigen with a Kd of 1 μM or less, and even more preferably 100 nM, 10 nM or even 1 nM or less. To further illustrate, the antibody-based antigen-binding domain specifically binds to a human HLA DR antigen. For instance, the antibody based antigen-binding domain can include a combination of a VH domain and a VL domain found in one of the clones taken from the list of MS-GPC-1, MS-GPC-2, MS-GPC-3, MS-GPC-4, MS-GPC-5, MS-GPC-6, MS-GPG7, MS-GPC-8, MS-GPC-10, MS-GPC-11, MS-GPC-14, MS-GPG15, MS-GPC-16, MS-GPC-8-1, MS-GPC-8-6, MS-GPC-8-9, MS-GPC8-10, MS-GPC,8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPG8-27-10 and MS-GPC-8-27-41.

In certain embodiments, the invention provides a composition including a polypeptide having at least one antibody-based antigen-binding domain with a binding specificity for a human MHC class II antigen, such as HLA DR, with a Kd of 1 μM or less, more preferably 100 nM, 10 nM or even 1 nM or less. The antibody based antigen-binding domain can be isolated by a method which includes isolation of VL and VH domains of human composition from a recombinant antibody library by ability to bind to human MHC class II antigen. The method may also include the further steps of:

a. generating a library of variants at least one of the CDR1, CDR2 and CDR3 sequences of one or both of the VL and VH domains, and

b. isolation of VL and VH domains from the library of variants by ability to bind to human MHC class II antigen with a Kd of 1 μM or less; and

c. (optionally) repeating steps (a) and (b) with further CDR1, CDR2 and CDR3 sequences.

In certain preferred embodiments, the antibody based antigen-binding domain of the subject polypeptides binds to the β-chain of HLA-DR, and even more preferably binds to an epitope of the first domain of the β-chain of HLA-DR.

One aspect of the present invention provides a multivalent composition of at least one polypeptide according to the invention is capable of leading to cell death of activated cells without requiring any further additional measures and with limited immunogenic side effects on the treated patient. Further, the multivalent composition comprising a polypeptide according to the invention has the capability of binding to at least one epitope on the target antigen, however, several epitope binding sites might be combined in one molecule. In a preferred embodiment the polypeptide is a multivalent composition comprising at least two monovalent antibody fragments selected from Fv, scFv, dsFv and Fab fragments, and further comprises a cross-linking moiety or moieties.

In a further preferred embodiment the polypeptide affects killing affects at least 50%, preferably at least 80%, of activated cells compared to killing of less than 15%, preferably less than 10%, of non-activated cells.

The compositions of the present invention can be used to treat a variety of cells, such as lymphoids and non-lymphoid cell, tough preferably those which express MHC class II antigens in the case of the latter.

In certain preferred embodiments, the subject compositions have an IC50 for inhibiting IL-2 secretion of 1 μM or less, and even more preferably 100 nM, 10 nM or even 1 nM or less.

In certain preferred embodiments, the subject compositions have an IC50 for inhibiting T cell proliferation of 1 μM or less, and even more preferably 100 nM, 10 nM or even 1 nM or less.

The composition of the present invention include polypeptides wherein the antibody based antigen-binding domain binds to one or more HLA-DR types selected from the group consisting of DR1-0101, DR2-15021, DR3-0301, DR4Dw4-0401, DR4Dw10-0402, DR4Dw14-0404, DR6-1302, DR6-1401, DR8-8031, DR9-9012, DRw53-B4*0101 and DRw52-B3*0101. In preferred embodiments, the the antigen binding domains of the subject compositions provide broad-DR reactivity, that is, the antigen-binding domain(s) of a given composition binds to epitopes on at least 3, and more preferably at least 5 or even 7 different of said HLA-DR types. In certain embodiments, the antigen binding domain(s) of a polypeptide(s) of the subject compositions binds to a plurality of HLA-DR types as to bind to HLA DR expressing cells for at least 60 percent of the human population, more preferably at least 75 percent, and even more preferably 85 percent of the human population.

In certain embodiments, the antibody based antigen-binding domain includes a combination of a VH domain and a VL domain, wherein the combination is found in one of the clones taken from the list MS-GPC-1, MS-GPC-2, MS-GPC-3, MS-GPG4, MS-GPC-5, MS-GPC-6, MS-GPC-7, MS-GPC-8, MS-GPC-10, MS-GPC-11, MS-GPC-14, MS-GPC-15, MS-GPC-16, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

In other embodiments, the antibody based antigen-binding domain includes a combination of HuCAL VH2 and HuCAL Vλ1, wherein the VH CDR3, VL CDR1 and VL CDR3 is found in one of the clones taken from the list of list MS-GPC-1, MS-GPC-4, MS-GPC-7, MS-GPC-8, MS-GPC-10, MS-GPC-11, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

In certain preferred embodiments, the antibody based antigen-binding domain includes a combination of HuCAL VH2 and HuCAL Vλ1, wherein the VH CDR3 sequence is taken from the consensus CDR3 sequence

nnnnRGnFDn

wherein each n independently represents any amino acid residue; and/or wherein the VL CDR3 sequence is taken from the consensus CDR3 sequence

QSYDnnnn

wherein each n independently represents any amino acid residue. Preferably, the VH CDR3 sequence is SPRYGAFDY and/or the VL CDR3 sequence is QSYDLIRH or QSYDMNVH.

In certain embodiments, the antibody based antigen-binding domain competes for antigen-binding with an antibody including a combination of HuCAL VH2 and HuCAL Vλ1. Preferably, the VH CDR3 sequence of the competing antibody is taken from the consensus CDR3 sequence

nnnnRGnFDn

each n independently represents any amino acid residue; and/or the VL CDR3 sequence is taken from the consensus CDR3 sequence

QSYDnnnn

each n independently represents any amino acid residue. In preferred embodiments, the VH CDR3 sequence is SPRYGAFDY and/or the VL CDR3 sequence is QSYDLIRH or QSYDMNVH.

The antibody based antigen-binding domains of the subject polypeptide can include a VL CDR1 sequence represented in the general formula

SGSnnNIGnNYVn

wherein each n independently represents any amino acid residue. In preferred embodiments, the CDR1 sequence is SGSESNIGNNYVQ.

In certain embodiments of the composition of the present invention, suppression of an immune response is brought about by or manifests itself in down-regulation of expression of said antigen expressed on the surface of said cell. Suppression of an immune response may also, or additionally, be brought about by or manifests itself in inhibition of the interaction between said cell and other cells, wherein said interaction would normally lead to an immune response, or the killing of the cells. In the instance of the latter, the killing can mediated by treating the cells expressing antigen with a plurality of antibody based antigen-binding domains, each of the antibody based antigen-binding domains being part of at least one multivalent polypeptide. In such instances, neither cytotoxic entities nor immunological mechanisms are needed to causes or leads to said killing.

In preferred embodiments of the subject polypeptide compositions, the killing affects at least 50%, preferably at least 75%, more preferably at least 85% of activated cells compared to killing of less than 30%, preferably less than 20%, more preferably less than 10% of non activated cells.

The compositions of the subject invention can also be characterized by inducing cell killing that is mediated by an innate, pre-programmed process of the cells. Where cell killing is an activity of the subject polypeptides, the killing is preferably non-apoptotic, and dependent on the action of non-caspase proteases, e.g., the the killing is independent of caspases that can be inhibited by zVAD-fmk or zDEVD-fmk.

In certain preferred embodiments, the composition of the present invention include antibody fragments selected from Fv, scFv, dsFv, Fab fragments, F(ab′)2, and mini-antibody fragments. The subject compositions can also include at least one full antibody, e.g., selected from the antibodies of classes IgG1, 2a, 2b, 3, 4, IgA, and IgM.

In certain instances, it may be desirable for subject compositions to include a cross-linking moiety or moieties, such that the antigen-binding sites are cross-linked to a polymer.

In preferred embodiments, the subject compositions can be formulated in a pharmaceutically acceptable carrier and/or diluent. For example, the subject invention specifically contemplates a pharmaceutical preparation including the subject antigen-binding composition in an amount sufficient to suppress an immune response in an animal, such as where said animal is human.

The present invention provides a pharmaceutical preparation including the subject antigen binding composition in an amount sufficient to inhibit IL-2 secretion in an animal, such as a human.

The present invention provides a pharmaceutical preparation including the subject antigen binding composition in an amount sufficient to inhibit T cell proliferation in an animal, such as where said animal is human.

The subject method also provides diagnostic compositions including the antigen binding compositions.

In still another embodiment, the subject method utilizes the antigen-binding compositions of present invention for preparing a pharmaceutical preparation for the treatment of animals, such as where said animals are human.

The present invention also provides nucleic acid including a protein coding sequence for polypeptide comprising at least one, antibody-based antigen-binding domain of human composition with a binding specificity for an antigen expressed on the surface of a cell. In preferred embodiments, treating cells expressing, the antigen with a polypeptide encoded by the nucleic acid causes or leads to suppression of an immune response, e.g., wherein the IC50 for the suppressive activity is 1 μM or lower, and even more preferably 100 nM, 10 nM or even 1 nM or less. Vectors including the protein coding sequence, and a transcriptional regulatory sequence operably linked thereto, are specifically contemplated, as are cells harboring the nucleic acid or the vector.

Such recombinant host cells can be used for the production of an immunosuppressive composition, by culturing the cells under conditions wherein the nucleic acid is expressed.

Another aspect of the present invention provides a method for suppressing activation and/or proliferation of a cell of the immune system, by treating the cell with a composition of a polypeptide including at least one antibody-based antigen-binding domain of human composition with a binding specificity for an antigen expressed on the surface of a cell. In preferred embodiments, treating cells expressing the antigen with a polypeptide encoded by the nucleic acid causes or leads to suppression of an immune response, e.g., wherein the IC50 for the suppressive activity is 1 μM or lower, and even more preferably 100 nM, 10 nM or even 1 nM or less. Similar methods can be used to inhibit IL-2 expression and/or cell-cell interactions involving cells of the immune system. In certain preferred embodiments, the subject method can be used for immunosuppressing a patient, e.g., by administering to the patient an effective amount of the antigen-binding composition.

Yet another aspect of the invention provides a method for killing a cell expressing an antigen on the surface of said cell comprising the step of treating the cell with a composition including a plurality of antibody based antigen-binding domains, e.g., as described above, wherein the antibody based antigen-binding domains are part of at least one multivalent polypeptide, and where neither cytotoxic entities nor immunological mechanisms are needed to cause or lead to said killing. Preferably, the antibody based antigen-binding domains bind to HLA DR.

Such methods can be used used for treating a disorder selected from rheumatoid arthritis, juvenile arthritis, multiple sclerosis, Grave's disease, insulin-dependent diabetes, narcolepsy, psoriasis, systemic lupus erythematosus, ankylosing spondylitis, transplant rejection, graft vs. host disease, Hashimoto's disease, myasthenia gravis, pemphigus vulgaris, glomerulonephritis, thyroiditis, pancreatitis, insulitis, primary biliary cirrhosis, irritable bowel disease and Sjogren syndrome.

In still another embodiment, there is provided a method to identify patients that can be treated with a antigen-binding composition of the present invention, including the steps of

a. Isolating cells from a patient;

b. Contacting said cells with a composition of the antigen-binding polypeptides; and

c. Measuring the degree of killing, immunosuppression, IL2 secretion or proliferation of said cells.

Such as method can be carried out using, e.g., a kit comprising

a. an antigen-binding composition of the present invention, and

b. a cross-linking moiety, and/or a detectable moiety or moieties (optionally including reagents and/or solutions to effect and/or detect binding to an antigen).

In yet other embodiments, the subject method provides a cytotoxic composition comprising the subject antigen-binding composition operably linked to a cytotoxic agent.

Another embodiment provides an immunogenic composition comprising the subject antigen-binding composition operably linked to an immunogenic agent.

Still another aspect of the invention provides a method to kill a cell expressing an antigen on the surface of said cell, comprising contacting said cell with an antigen-binding composition of the present invention operably linked to a cytotoxic or immunogenic agent. In this regard, the invention also specifically contemplates the use of the subject antigen-binding compositions operably linked to a cytotoxic or immunogenic agent for the preparation of a pharmaceutical composition for the treatment of animals.

Yet another aspect of the present invention provides a method for conducting a pharmaceutical business comprising:

(i) isolating one or more antibody based antigen-binding domains that bind to MHC class II expressed on the surface of human cells with a Kd of 1 μM or less;

(ii) generating a composition comprising said antibody based antigen-binding domains, which composition is immunosuppressant with an IC50 of 100 nM or less;

(iii) conducting therapeutic profiling of said composition for efficacy and toxicity in animals;

(iv) preparing a package insert describing the use of said composition for immunosuppression therapy; and

(v) marketing said composition for use as an immunosuppressant.

Another embodiment for a method of conducting a life science business includes:

(i) isolating one or more antibody based antigen-binding domains that bind to MHC class II expressed on the surface of human cells with a Kd of 1 μM or less;

(ii) generating a composition comprising said antibody based antigen-binding domains, which composition is immunosuppressant with an IC50 of 100 nM or less;

(iii) licensing, jointly developing or selling, to a third party, the rights for selling said composition.

According to the subject business methods, the antibody based antigen-binding domain can be isolated by a method which includes

a. isolation of VL and VH domains of human composition from a recombinant antibody library by ability to bind to HLA DR,

b. generating a library of variants at least one of the CDR1, CDR2 and CDR3 sequences of one or both of the VL and VH domains, and

c. isolation of VL and VH domains from the library of variants by ability to bind to HLA DR with a Kd of 1 μM or less.

According to the subject business methods, the antigen-binding domain can be a combination of VH and VL domains found in the clones taken from the list of MS-GPC-1, MS-GPC-2, MS-GPC-3, MS-GPC-4, MS-GPC-5, MS-GPC-6, MS-GPC-7, MS-GPC-8, MS-GPC-10, MS-GPC-11, MS-GPC-14, MS-GPC-15, MS-GPC-16, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

As used herein, the term “peptide” relates to molecules consisting of one or more chains of multiple, i. e. two or more, amino adds linked via peptide bonds.

The term “protein” refers to peptides where at least part of the peptide has or is able to acquire a defined three-dimensional arrangement by forming secondary, tertiary, or quaternary structures within and/or between its peptide chain(s). This definition comprises proteins such as naturally occurring or at least partially artificial proteins, as well as fragments or domains of whole proteins, as long as these fragments or domains are able to acquire a defined three-dimensional arrangement as described above.

The term “polypeptide” is used interchangeably to refer to peptides-and/or proteins. Moreover, the terms “polypeptide ” and “protein”, as the context will admit, include multi-chain protein complexes, such as immunoglobulin polypeptides having separate heavy and light chains.

In this context, a “polypeptide comprising at least one antibody-based antigen-binding domain” refers to an immunoglobulin (e.g. IgG, IgA or IgM molecules or antibody) or to a functional fragment thereof. The term “functional fragment”, or “antibody fragment” as it may be occasionally referred to, refers to a fragment of an immunoglobulin which retains the antigen-binding moiety of an immunoglobulin. Functional immunoglobulin fragments according to the present invention may be Fv (Skerra and Plückthun, 1988), scFv (Bird et al., 1988; Huston et al., 1988), disulfide-linked Fv (Glockshuber et al., 1992; Brinkmann et al., 1993), Fab, F(ab′)₂ fragments or other fragments well-known to the practitioner skilled in the art, which comprise the variable domains of an immunoglobulin or functional immunoglobulin fragment.

Examples of polypeptides consisting of one chain are single-chain Fv antibody fragments, and examples for polypeptides consisting of more chains are Fab antibody fragments.

The term “antibody” as used herein, unless indicated otherwise, is used broadly to refer to both antibody molecules and a variety of antibody derived molecules. Such antibody derived molecules comprise at least one variable region (either a heavy chain of light chain variable region) and include such fragments as described above, as well as individual antibody light chains, individual antibody heavy chains, chimeric fusions between antibody chains and other molecules, and the like.

For the purposes of this application, “valent” refers to the number of antigen binding sites the subject polypeptide possess. Thus, a bivalent polypeptide refers to a polypeptide with two binding sites. The term “multivalent polypeptide” encompasses bivalent, trivalent, tetravalent, etc. forms of the polypeptide.

The “antigen-binding site” of an immunoglobulin molecule refers to that portion of the molecule that is necessary for binding specifically to an antigen. An antigen binding site preferably binds to an antigen with a Kd of 1 μM or less, and more preferably less than 100 nM, 10 nM or even 1 nM in certain instances. Binding specifically to an antigen is intended to include binding to the antigen which significantly higher affinity than binding to any other antigen.

The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Accordingly, an “antibody-based antigen-binding domain” refers to polypeptide or polypeptides which form an antigen-binding site retaining at least some of the structural features of an antibody, such as at least one CDR sequence. In certain preferred embodiments, antibody-based antigen-binding domain includes sufficient structure to be considered a variable domain, such as three CDR regions and interspersed framework regions. Antibody-based antigen-binding domain can be formed single polypeptide chains corresponding to VH or VL sequences, or by intermolecular or intramolecular association of VH and VL sequences.

The term “recombinant antibody library” describes a collection of display packages, e.g., biological particles, which each have (a) genetic information for expressing at least one antigen binding domain on the surface of the particle, and (b) genetic information for providing the particle with the ability to replicate. For instance, the package can display a fusion protein including an antigen binding domain. The antigen binding domain portion of the fusion protein is presented by the display package in a context which permits the antigen binding domain to bind to a target epitope that is contacted with the display package. The display package will generally be derived from a system that allows the sampling of very large variegated antibody libraries. The display package can be, for example, derived from vegetative bacterial cells, bacterial spores, and bacterial viruses.

In an exemplary embodiment of the present invention, the display package is a phage particle which comprises a peptide fusion coat protein that includes the amino acid sequence of a test antigen binding domains. Thus, a library of replicable phage vectors, especially phagemids (as defined herein), encoding a library of peptide fusion coat proteins is generated and used to transform suitable host cells. Phage particles formed from the chimeric protein can be separated by affinity, selection based on the ability of the antigen binding site associated with a particular phage particle to specifically bind a target eptipope. In a preferred embodiment, each individual phage particle of the library includes a copy of the corresponding phagemid encoding the peptide fusion coat protein displayed on the surface of that package. Exemplary phage for generating the present variegated peptide libraries include M13, f1, fd, If1, Ike, Xf, Pf1, Pf3, λ, T4, T7, P2, P4, φX-174, MS2 and f2.

The term “generating a library of variants of at least one of the CDR1, CDR2 and CDR3” refers to a process of generating a library of variant antigen binding sites in which the members of the library differ by one or more changes in CDR sequences, e.g., not FR sequences. Such libraries can be generated by random or semi-random mutagenesis of one or more CDR sequences from a selected antigen binding site.

As used herein, an “antibody-based antigen-binding domain of human composition” preferably means a polypeptide comprising at least an antibody VH domain and an antibody VL domain, wherein a homology search in a database of protein sequences comprising immunoglobulin sequences results for both the VH and the VL domain in an immunoglobulin domain of human origin as hit with the highest degree of sequence identity. Such a homology search may be a BLAST search, e.g. by accessing sequence databases available through the National Center for Biological Information and performing a “BasicBLAST” search using the “blastp” routine. See also Altschul et al. (1990) J Mol Biol 215:403-410. Preferably, such a composition does not result in an adverse immune response thereto when administered to a human recipient. In certain preferred embodiments, the subject antigen-binding domains of human composition include the framework regions of native human immunoglobulins, as may be cloned from activated human B cells, though not necessarily all of the CDRs of a native human antibody.

As used herein, the term mini-antibody fragment” means a multivalent antibody fragment comprising at least two antigen-binding domains multimerized by self-associating domains fused to each of said domains (Pack, 1994), e.g. dimers comprising two scFv fragments, each fused to a self-associating dimerization domain. Dimerization domains, which are particularly preferred, include those derived from a leucine zipper (Pack and Plückthun, 1992) or helix-turn-helix motif (Pack et al., 1993).

As used herein, “activated cells” means cells of a certain population of interest, which are not resting. Activation might be caused by antigens, mitogens (e.g., lipopoysaccharide, phytohemagglutinine) or cytokines (e.g., interferon gamma). Preferably, said activation occurs during the stimulation of resting T and B cells in the course of the generation of an immune response. Activated cells may be certain lymphoid tumour cells. Preferably, activated cells are characterised by the feature of MHC class II molecules expressed on the cell surface and one or more additional feature including increased cell size, cell division, DNA replication, expression of CD45 or CD11 and production/secretion of immunoglobulin.

As used herein, “non-activated cells” means cells of a population of interest, the vast majority of which are resting and non-dividing. Said non-activated cells may include resting B cells as purified from healthy human blood. Such cells can, preferably, be characterised by lack or reduced level of MHC class II molecules expressed on the cell surface and lack or reduced level of one or more additional features including increased cell size, cell division, DNA replication, expression of CD45 or CD11 and production/secretion of immunoglobulin.

“Lymphoid cells” when used in reference to a cell line or a cell, means that the cell line or cell is derived from the lymphoid lineage and includes cells of both the B and the T lymphocyte lineages, and the macrophage lineage.

“Non lymphoid cells and express MHC class II” means cells other than lymphoid cells that express MHC class II molecules during a pathological inflammatory response. For example, said cells may include synovial cells, endothelial cells, thyroid stromal cells and glial cells and it may also comprise genetically altered cells capable of expressing MHC-class II molecules.

As used herein, the term “first domain of the alpha-chain of HLA-DR” means the N-terminal domain of the alpha-chain.

As used herein, the term “first domain of the beta-chain of HLA-DR” means the N-terminal domain of the beta-chain.

As used herein, the term “modulation of the immune response” relates to the changes in activity of the immune response of an individual or to changes of an in vitro system resembling parts of an immune system. Said changes in activity are causing or leading to immunosuppression.

The term “immunosuppress” refers to the prevention or diminution of the immune response, as by irradiation or by administration of antimetabolites, antilymphocyte serum, or specific antibody.

The term “immune response” refers to any response of the immune system, or a cell forming part of the immune system (lymphocytes, granulocytes, macrophages, etc), to an antigenic stimulus, including, without limitation, antibody production, cell-mediated immunity, and immunological tolerance.

As used herein, the term “IC50” with respect immunosuppression, refers to the concentration of the subject compositions which produces 50% of its maximum response or effect, such as inhibition of an immune response, such as may be manifest by T-cell activation (cellular response) or B-cell activation (humoral response).

The terms “apoptosis” and “apoptotic activity” refer to the form of cell death in mammals that is accompanied by one or more characteristic morphological and biochemical features, including nuclear and condensation of cytoplasm, chromatin aggregation, loss of plasma membrane microvilli, partition of cytoplasm and nucleus into membrane bound vesicles (apoptotic bodies) which contain ribosomes, morphologically intact mitochondria and nuclear material, degradation of chromosomal DNA or loss of mitochondrial function. Apoptosis follows a very stringent time course and is executed by caspases, a specific group of proteases. Apoptotic activity can be determined and measured, for instance, by cell viability assays, Annexin V staining or caspase inhibition assays. Apoptosis can be induced using a cross-linking antibody such as anti-CD95 as described in Example H.

The term “innate preprogrammed process” refers to a process that, once it is started, follows an autonomous cascade of mechanisms within a cell, which does not require any further auxiliary support from the environment of said cell in order to complete the process.

As used herein, the term “HuCAL” refers to a fully synthetic human combinatorial antibody library as described in Knappik et al. (2000).

As used herein, the term “CDR3” refers to the third complementarity-determining region of the VH and VL domains of antibodies or fragments thereof, wherein the VH CDR3 covers positions 95 to 102 (possible insertions after positions 100 listed as 100a to 100 z), and VL CDR3 positions 89 to 96 (possible insertions in Vλ after position 95 listed as 95a to 95c) (see Knappik et al., 2000).

The term “variable region” as used herein in reference to immunoglobulin molecules has the ordinary meaning given to the term by the person of ordinary skill in the act of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al “Sequences of Proteins of Immunological Interest: 5th Edition” U.S. Department of Health and Human Services, U.S. Government Printing Office (1991).

As used herein, the term “hybridises under stringent conditions” is intended to describe conditions for hybridisation and washing under which nucleotide sequences at least 60% homologous to each other typically remain hybridised to each other. Preferably, the conditions are such that at least sequences at least 65%, more preferably at least 70%, and even more preferably at least 75% homologous to each other typically remain hybridised to each other. Such stringent conditions are known to those skilled in the art and can be found in Current Protocols in Molecular Biology, John Wiley & Sons, New York (1999), 6.3.1-6.3.6. A preferred, non-limiting example of stringent hybridisation conditions is hybridisation in 6× sodium chloride/sodium citrate (SSC) at about 45° C., followed by one or more washes in 0.2×SSC, 0.1 % SDS at 50°-65° C.

A “protein coding sequence” or a sequence which “encodes” a particular polypeptide or peptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences which encode a polypeptide, as the term is typically used, as well as DNA sequences which are transcribed into inhibitory antisense molecules.

As used herein, the term “transfection” means the introduction of a heterologous nucleic acid, e.g., an expression vector, into a recipient cell by nucleic acid-mediated gene transfer. “Transient transfection” refers to cases where exogenous DNA does not integrate into the genome of a transfected cell, e.g., where episomal DNA is transcribed into mRNA and translated into protein. A cell has been “stably transfected” with a nucleic acid construct when the nucleic acid construct is capable of being inherited by daughter cells.

“Expression vector” refers to a replicable DNA construct used to express DNA which encodes the desired protein and which includes a transcriptional unit comprising an assembly of (1) agent(s) having a regulatory role in gene expression, for example, promoters, operators, or enhancers, operatively linked to (2) a DNA sequence encoding a desired protein (such as a polypeptide of the present invention) which is transcribed into mRNA and translated into protein, and (3) appropriate transcription and translation initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

In the expression vectors, regulatory elements controlling transcription or translation can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

“Transcriptional regulatory sequence” is a generic term used throughout the specification to refer to DNA sequences, such as initiation signals, enhancers, and promoters and the like which induce or control transcription of protein coding sequences with which they are operably linked. It will be understood that a recombinant gene can be under the control of transcriptional regulatory sequences which are the same or which are different from those sequences which control transcription of the naturally-occurring form of the gene, if any.

“Operably linked” when describing the relationship between two DNA regions simply means that they are functionally related to each other. For example, a promoter or other transcriptional regulatory sequence is operably linked to a coding sequence if it controls the transcription of the coding sequence.

As used herein, the term “fusion protein” is art recognized and refer to a chimeric protein which is at least initially expressed as single chain protein comprised of amino acid sequences derived from two or more different proteins, e.g., the fusion protein is a gene product of a fusion gene.

As used herein the term “animal” refers to mammals, preferably mammals such as humans. Likewise, a “patient” or “subject to be treated by the method of the invention can mean either a human or non-human animal.

According to the methods of the invention, the peptide may be administered in a pharmaceutically acceptable composition. In general, pharmaceutically-acceptable carriers for monodonal antibodies, antibody fragments, and peptides are well-known to those of ordinary skill in the art. As used herein, the term “pharmaceutically acceptable carrier” includes any and all solvents, dispersion media, coatings, antibacterial and antifungal agents, isotonic and absorption delaying agents and the like. In preferred embodiments, the subject carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitoneal, with intravenous being preferred.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds, e.g., the subject polypeptides, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are, formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Upon formulation, solutions can be administered in a manner compatible with the dosage formulation and in such amount as is therapeutically effective. The formulations are easily administered in a variety of dosage forms such as injectable solutions, drug release capsules and the like.

As used herein, the term “prophylactic or therapeutic” treatment refers to administration to the host of the medical condition, e.g., to cause immunosuppression. If it is administered prior to exposure to the condition, the treatment is prophylactic, whereas if administered after infection or initiation of the disease, the treatment is therapeutic.

The polypeptide according to the invention is comprising at least one antibody-based antigen-binding domain of human composition with binding specificity for a human MHC class II antigen, wherein binding of said polypeptide to said antigen expressed on the surface of a cell causes or leads to a modulation of the immune response.

The present invention further relates to a pharmaceutical composition containing at least one antigen-binding polypeptide according to the invention, optionally together with a pharmaceutical acceptable carrier and/or diluent. The polypeptide according to the invention is preferably used for preparing a pharmaceutical composition for treating animals, preferably humans. The polypeptide according to the invention is preferably useful for the treatment or prevention of a condition characterised by MHC class II-mediated activation of T and/or B cells. In a further preferred embodiment said treatment is the treatment or prevention of a condition characterised by expression of MHC class II expression at pathological sites of inflammation. In a further preferred embodiment said treatment is the treatment or prevention of diseases of the immune system.

In a preferred embodiment the antigen-binding compositions of the invention can be used in the treatment of diseases of the immune system including conditions such as rheumatoid arthritis, juvenile arthritis, multiple sclerosis, Grave's disease, narcolepsy, psoriasis, systemic lupus erythematosus, transplant rejection, graft vs. host disease, Hashimoto's disease, myasthenia gravis, pemphigus vulgaris, glomerulonephritis, thyroiditis, insulitis, primary biliary cirrhosis, irritable bowel disease and Sjogren syndrome.

The invention further relates to a diagnostic composition containing at least one polypeptide and/or nucleic acid according to the invention optionally together with further reagents, such as buffers, for performing the diagnosis.

Additionally, the present invention relates to a kit comprising (i) a polypeptide according to the present invention, (ii) a detectable moiety or moieties, and (iii) reagents and/or solutions to effect and/or detect binding of (i) to an antigen.

### BRIEF DESCRIPTION OF THE DRAWINGS

FIG. 1

a. Specificity of the anti-HLA-DR antibody fragments: Binding of MS-GPC-8-27-7, MS-GPC-8-27-10, MS-GPC-8-6-13, MS-GPC-8-27-41, MS-GPC-8-6-47, MS-GPC-8-10-57, MS-GPC-8-6-27, MS-GPC-8 and MS-GPC-8-6 to HLA-DR protein, negative control proteins (BSA, testosterone-BSA, lysozyme and human apotransferrin), and an empty microtiter plate well (plastic). Specificity was assessed using standard ELISA procedures.

b. Specificity of the anti-HLA-DR antibody fragments MS-GPC-1, 2, 3, 4, 5, 6, 7, 8, 10, 11, 14, 15 & 16 isolated from the HuCAL library to HLA-DR protein, a mouse-human chimeric HLA protein and negative control proteins (lysozyme, transferrin, BSA and human gamma-globulin). Specificity was assessed using standard ELISA procedures. A non-related antibody fragment (irr. scFv) was used as control.

FIG. 2

Reactivity of the anti-HLA-DR antibody fragments MS-GPC-1, 2, 3, 4, 5, 6, 7, 8, 10, 11, 14, 15 & 16 and of the IgG forms of MS-GPC-8, MS-GPC-8-10-57, MS-GPC-8-27-41 & MS-GPC-8-6-17 to various cell lines expressing MHC class II molecules. “+” represents strong reactivity as detected using standard immunofluorescence procedure. “±” represents weak reactivity and “−” represents no detected reactivity between an anti-HLA-DR antibody fragment or IgG and a particular cell line.

FIG. 3

Viability of tumor cells in the presence of monovalent and cross-linked anti-HLA-DR antibody fragments as assessed by trypan blue staining. Viability of GRANTA-519 cells was assessed after 4 h incubation with anti-HLA-DR antibody fragments (MS-GPC-1, 6, 8 and 10) with and without ant-FLAG M2 mAb as cross-linking agent.

FIG. 4

Scatter plots and fitted logistic curves of data from Table 5 showing improved killing efficiency of 50 nM solutions of the IgG form of the human antibody fragments of the invention treated compared to treatment with 200 nM solutions of murine antibodies. Open circles represent data for cell lines treated with the murine antibodies L243 and 8D1 and closed circles for human antibodies MS-GPC-8, MS-GPC-8-27-41, MS-GPC-8-10-57 and MS-GPC-6-13. Fitted logistic curves for human (solid) and mouse (dashed) mAb cell killing data show the overall superiority of the treatment with human mAbs at 50 nM compared to the mouse mAbs despite treatment at a final concentration of 200 nM.

FIG. 5

Killing of activated versus non-activated cells. MHH-PREB-1 cells are activated with Lipopolysaccharide, Interferon-gamma and phyto-hemagglutin, and subsequently incubated for 4 h with 0.07 to 3300 nM of the IgG forms of the anti-HLA-DR antibody fragments MS-GPC-8-10-57 and MS-GPC-8-27-41. No loss of viability in the control non-activated MHH-PREB-1 cells is seen.

FIG. 6

Killing efficiency of control (no antibody, non-cytotoxic murine IgG 1oF12; light grey), and human (MS-GPC-8, MS-GPC-8-10-57 & MS-GPC-8-27-41; dark grey) IgG forms of anti-HLA-DR antibody fragments against CLL cells isolated from patients. Left panel, box-plot display of viability data from 10 patient resting cell cultures against antibodies after incubation for four (h4) and twenty four hours (h24). Right panel box-plot display of viability data from 6 patient activated cell cultures against antibodies after incubation for four (h4) and twenty four hours (h24).

FIG. 7

Concentration dependent cell viability for certain anti-HLA-DR antibody fragments of the invention. Vertical lines indicate the EC50 value estimated by logistic non-linear regression on replica data obtained for each of the antibody fragments. a) Killing curves of cross-linked bivalent anti-HLA-DR antibody F(ab) fragment dimers MS-GPC-10 (circles and solid line), MS-GPC-8 (triangles and dashed line) and MS-GPC-1 (crosses and dotted line). b) Killing curves of cross-linked bivalent anti-HLA-DR antibody (Fab) fragment dimers MS-GPC-8-17 (circles and solid line), and murine IgGs 8D1 (triangles and dashed line) and L243 (crosses and dotted line). c) Killing curves of cross-linked bivalent anti-HLA-DR antibody (Fab) fragment dimers GPC-8-6-2 (triangles and dashed line), and murine IgGs 8D1 (circles and solid line) and L243 (crosses and dotted line). d) Killing curves of IgG forms of human anti-HLA-DR antibody fragments MS-GPC-8-10-57 (crosses and dotted line), MS-GPC-8-27-41 (exes and dash-dot line), and murine IgGs 8D1 (circles and solid line) and L243 (triangles and dashed line). All concentrations are given in nM of the bivalent agent (IgG or crosslinked (Fab) dimer).

FIG. 8

a. Incubation of Priess cells with the anti-HLA-DR antibody fragment MS-GPC-8, cross-linked using the anti-FLAG M2 mAb, shows more rapid killing than a culture of Priess cells induced into apoptosis using anti-CD95 mAb. An Annexin V/PI staining technique identifies necrotic cells by Annexin V positive and PI positive staining.

b. Incubation of Priess cells with the anti-HLA-DR antibody fragment MS-GPC-8, cross-linked using the anti-FLAG M2 mAb, shows little evidence of an apoptotic mechanism compared to an apoptotic culture of Priess cells induced using anti-CD95 mAb. An Annexin V/PI staining technique identifies apoptotic cells by Annexin V positive and PI negative staining.

FIG. 9

a. Immunosuppressive properties of the IgG forms of the anti-HLA-DR antibody fragments MS-GPC-8-10-57, MS-GPC-8-27-41 & MS-GPC-8-6-13 using an assay to determine inhibition of IL-2 secretion from T-hybridoma cells.

b. Immunosuppressive properties of the monovalent Fab forms of the anti-HLA-DR antibody fragments MS-GPC-8-27-41 & MS-GPC-8-6-19 using an assay to determine inhibition of IL-2 secretion from T-hybridoma cells.

Concentrations for the IgG forms (bivalent) are represented as molar concentrations, while those for the Fab forms (monovalent) are expressed in terms of half the concentration of the Fab form to enable direct comparison to concentrations of IgG forms.

FIG. 10

Immunosuppressive properties of the IgG forms of the anti-HLA-DR antibody fragments MS-GPC-8-10-57 and MS-GPC-8-27-41 in an assay to determine inhibition of T cell proliferation.

FIG. 11

Vector map and sequence of scFv phage display vector pMORPH13_scFv.

The vector pMORPH13_scFv is a phagemid vector comprising a gene encoding a fusion between the C-terminal domain of the gene III protein of filamentous phage and a HUCAL scFv. In FIG. 11, a vector comprising a model scFv gene (combination of VH1A and Vλ3 (Knappik et al., 2000) is shown.

The original HUCAL master genes (Knappik et al. (2000): see FIG. 3 therein) have been constructed with their authentic Nermini: VH1A, VH1B, VH2, VH4 and VH6 with Q (=CAG) as the first amino acid. VH3 and VH5 with E (=GAA) as the first amino acid. Vector pMORPH13_scFv comprises the short FLAG peptide sequence (DYKD) fused to the VH chain, and thus all HuCAL VH chains in, and directly derived from, this vector have E (=GAA) at the first position (e.g. in pMx7_FS vector, see FIG. 12).

FIG. 12

Vector map and sequence of scFv expression vector pMx7_FS_{—}5D2.

The expression vector pMx7_FS_{—}5D2 leads to the expression of HuCAL scFv fragments (in FIG. 12, the vector comprises a gene encoding a “dummy” antibody fragment called “5D2”) when VH-CH1 is fused to a combination of a FLAG tag (Hopp et al., 1988; Knappik and Plückthun, 1994) and a STREP tag II (WSHPQFEK) (IBA GmbH, Göttingen, Germany; see: Schmidt and Skerra, 1993; Schmidt and Skerra, 1994; Schmidt et al., 1996; Voss and Skerra, 1997).

FIG. 13

Vector map and sequence of Fab expression vector pMx9_Fab_GPC-8.

The expression vector pMx9_Fab_GPC8 leads to the expression of HuCAL Fab fragments (in FIG. 13, the vector comprises the Fab fragment MS-GPC8) when VH-CH1 is fused to a combination of a FLAG tag (Hopp et al., 1988; Knappik and Plückthun, 1994) and a STREP tag II (WSHPQFEK) (IBA GmbH, Göttingen, Germany; see: Schmidt and Skerra, 1993; Schmidt and Skerra, 1994; Schmidt et al., 1996; Voss and Skerra, 1997).

In pMx9_Fab vectors, the HuCAL Fab fragments cloned from the scFv fragments (see figure caption of FIG. 11) do not have the short FLAG peptide sequence (DYKD) fused to the VH chain, and all HuCAL VH chains in, and directly derived from, that vector have Q (=CAG) at the first position.

FIG. 14

Vector map and sequence of Fab phage display vector pMORPH18_Fab_GPC8.

The derivatives of vector pMORPH18 are phagemid vectors comprising a gene encoding a fusion between the C-terminal domain of the gene III protein of filamentous phage and the VH-CH1 chain of a HuCAL antibody. Additionally, the vector comprises the separately encoded VL-CL chain. In FIG. 14, a vector comprising the Fab fragment MS-GPC-8 is shown.

In pMORPH18_Fab vectors, the HuCAL Fab fragments cloned from the scFv fragments (see figure caption of FIG. 11) do not have the short FLAG peptide sequence (DYKD) fused to the VH chain, and all HUCAL VH chains in, and directly derived from, that vector have Q (=CAG) at the first position.

FIG. 15

Amino acid sequences of VH and VL domains of MS-GPC-1, 2, 3, 4, 5, 6, 7, 8, 10, 11, 14, 15 & 16, and MS-GPC-8-6, MS-GPC-8-10, MS-GPC-8-17, MS-GPC-8-27, MS-GPC-8-6-13, MS-GPC-8-10-57, and MS-GPC-8-27-41.

The sequences in FIG. 15 show amino acid 1 of VH as constructed in the original HuCAL master genes (Knappik et al. (2000): see FIG. 3 therein). In scFv constructs, as described in this application, amino acid 1 of VH is always E (see figure caption of FIG. 11), in Fab constructs as described in this application, amino acid 1 of VH is always Q (see figure caption of FIG. 13).

### DETAILED DESCRIPTION OF THE INVENTION

The following examples illustrate the invention.

### EXAMPLES

All buffers, solutions or procedures without explicit reference can be found in standard textbooks, for example Current Protocols of Immunology (1997 and 1999) or Sambrook et al., 1989 (this reference has no publisher). Where not given otherwise, all materials were purchased from Sigma, Deisenhofen, DE, or Merck, Darmstadt, DE, or sources are given in the literature cited. Hybridoma cell lines LB3.1 and L243 were obtained from LGC Reference Materials, Middlesex, UK; data on antibody 8D1 were generously supplied by Dr. Matyas Sandor, University of Michigan, Madison, Wis., USA.

1. Preparation of a Human Antigen

To demonstrate that we could identify cytotoxic antigen-binding domains of human composition, we first prepared a purified form of a human antigen, the human MHC class II DR protein (DRA*0101/DRB1*0401) from PRIESS cells (Gorga et al., 1984; Gorga et al., 1986; Gorga et al., 1987; Stem et al., 1992) as follows.

First, PRIESS cells (ECACC, Salisbury UK) were cultured in RPMI and 10% fetal calf serum (FCS) using standard conditions, and 10¹⁰ cells were lysed in 200 ml phosphate buffered saline (PBS) (pH 7.5) containing 1% NP-40 (BDH, Poole, UK), 25 mM iodoacetamide, 1 mM phenylmethylsulfonylfluoride (PMSF) and 10 mg/l each of the protease inhibitors chymostatin, antipain, pepstatin A, soybean trypsin inhibitor and leupeptin. The lysate was centrifuged at 10.000 g (30 minutes, 4° C.) and the resulting supernatant was supplemented with 40 ml of an aqueous solution containing 5% sodium deoxycholate, 5 mM iodoacetamide and 10 mg/l each of the above protease inhibitors and centrifuged at 100.000 g for two hours (4° C.). To remove material that bound non-specifically and endogenous antibodies, the resulting supernatant was made 0.2 mM with PMSF and passed overnight (4° C.) through a rabbit serum affigel-10 column (5 ml; for preparation, rabbit serum (Charles River, Wilmington, Mass., USA) was incubated with Affigel 10 (BioRad, Munich, DE) at a volume ratio of 3:1 and washed following manufacturer's directions) followed by a Protein G Sepharose Fast Flow column (2 ml; Pharmacia) using a flow rate of 0.2 ml/min.

Second, the pre-treated lysate was batch incubated with 5 ml Protein G Sepharose Fast Flow beads coupled to the murine anti-HLA-DR antibody LB3.1 (obtained by Protein G-Sepharose FF (Pharmacia) affinity chromatography of a supernatant of hybridoma cell line LB3.1) (Stern et al., 1993) overnight at 4° C. using gentle mixing, and then transferred into a small column which was then washed extensively with three solutions: (1) 100 ml of a solution consisting of 50 mM Tris/HCl (pH 8.0), 150 mM NaCl, 0.5% NP-40, 0.5% sodium deoxycholate, 10% glycerol and 0.03% sodium azide at a flow rate of 0.6 ml/min). (2) 25 ml of a solution consisting of 50 mM Tris/HCl (pH 9.0); 0.5 M NaCl, 0.5 % NP-40, 0.5°/sodium deoxycholate, 10% glycerol and 0.03% sodium azide at a flow rate of 0.9 ml/min; (3) 25 ml of a solution consisting of 2 mM Tris/HCl (pH 8.0), 1% octyl-β-D-glucopyranoside, 10% glycerol and 0.03% sodium azide at a flow rate of 0.9 ml/min.

Third, MHC class II DR protein (DRA*0101/DRB1*0401) was eluted using 15 ml of a solution consisting of 50 mM diethylamine/HCl (pH 11.5), 150 mM NaCl, 1 mM EDTA, 1 mM EGTA, 1% octyl-β-D-glucopyranoside (Alexis Corp., Lausen, CH), 10% glycerol, 10 mM iodoacetamide and 0.03% sodium azide at a flow rate of 0.4 ml/min. 800 μl fractions were immediately neutralised with 100 μl M Tris/HCl (pH 6.8), 150 mM NaCl and 1% octyl-β-D-glucopyranoside. The incubation of the lysate with LB3.1-Protein G Sepharose Fast Flow beads was repeated until the lysate was exhausted of MHC protein. Pure eluted fractions of the MHC class II DR protein (as analyzed by SDS-PAGE) were pooled and concentrated to 1.0-1.3 g/l using Vivaspin concentrators (Greiner, Solingen, DE) with a 30 kDa molecular weight cut-off. Approximately 1 mg of the MHC class II DR preparation was re-buffered with PBS containing 1% octyl-β-D-glucopyranoside using the same Vivaspin concentrator to enable direct coupling of the protein to BIAcore CM5 chips.

2. Screening of HuCAL

2.1. Introduction

We identified certain antigen binding antibody fragments of human composition (MS-GPC-1, 2, 3, 4, 5, 6, 7, 8, 10, 11, 14, 15 & 16) against the human antigen (DRA*0101/DRB1*0401) from a human antibody library based on a novel concept that has been recently developed (Knappik et al., 2000). A consensus framework resulting in a total of 49 different frameworks here represents each of the VH- and VL-subfamilies frequently used in human immune responses. These master genes were designed to take into account and eliminate unfavorable residues promoting protein aggregation as well as to create unique restriction sites leading to modular composition of the genes. In HuCAL-scFv, both the VH- and VL-CDR3 encoding regions of the 49 master genes were randomized.

2.2. Phagemid Rescue, Phage Amplification and Purification

The HuCAL-scFv (Knappik et al., 2000) library, cloned into a phagemid-based phage display vector pMORPH13_scFv (see FIG. 11), in E. coli TG-1 was amplified in 2×TY medium containing 34 μg/ml chloramphenicol and 1% glucose (2×TY-CG). After helper phage infection (VCSM13) at 37° C. at an OD₆₀₀ of about 0.5, centrifugation and resuspension in 2×TY/34 μg/ml chloramphenicol/50 μg/ml kanamycin/0.1 mM IPTG, cells were grown overnight at 30° C. Phage were PEG-precipitated from the supernatant (Ausubel et al., 1998), resuspended in PBS/20% glycerol and stored at −80° C. Phage amplification between two panning rounds was conducted as follows: mid-log phase TG1-cells were infected with eluted phage and plated onto LB-agar supplemented with 1% of glucose and 34 μg/ml of chloramphenicol. After overnight incubation at 30° C. colonies were scraped off, adjusted to an OD₆₀₀ of 0.5 and helper phage added as described above.

2.3. Manual Solid Phase Panning

Wells of MaxiSorp™ microtiterplates (Nunc, Roskilde, DK) were coated with MHC-class II DRA*0101/DRB1*0401 (prepared as above) dissolved in PBS (2 μg/well). After blocking with 5% non-fat dried milk in PBS, 1-5×10¹² HuCAL-scFv phage purified as above were added for 1 h at 20° C. After several washing steps, bound phages were eluted by pH-elution with 100 mM triethylamine and subsequent neutralization with 1M TRIS-Cl pH 7.0. Three rounds of panning were performed with phage amplification conducted between each round as described above.

2.4. Mixed Solid Phase/Whole Cell Panning

Three rounds of panning and phage amplification were performed as described in 2.3. and 2.2. with the exception that in the second round between 1×10⁷ and 5×10⁷ PRIESS cells in 1 ml PBS/10% FCS were used in 10 ml Falcon tubes for whole cell panning. After incubation for 1 h at 20° C. with the phage preparation, the cell suspension was centrifuged (2000 rpm for 3 min) to remove non-binding phage, the cells were washed three times with 10 ml PBS, each time followed by centrifugation as described. Phage that specifically bound to the cells were eluted off by pH-elution using 100 mM HCl. Alternatively, binding phage could be amplified by directly adding E. coli to the suspension after triethlyamine treatment (100 mM) and subsequent neutralization.

2.5 Identification of HLA-DR Binding scFv Fragments

Clones obtained after three rounds of solid phase panning (2.3) or mixed solid phase/whole:.cell panning (02.4) were screened by FACS analysis on PRIESS cells for binding to HLA-DR on the cell surface. For expression, the scFv fragments were cloned via XbaI/EcoRI into pMx7_FS as expression vector (see FIG. 12). Expression conditions are shown below in example 3.2.

Aliquots of 10⁶ Priess cells were transferred at 4° C. into wells of a 96-well microfiterplate. ScFv in blocking buffer (PBS/5% FCS) were added for 60 min and detected using an anti-FLAG M2 antibody (Kodak) (1:5000 dilution) followed by a polyclonal goat anti-mouse IgG antibody-R-Phycoerythrin-conjugate (Jackson ImmunoResearch, West Grove, Pa., USA, Cat. No. 115-116-146, F(ab′)₂ fragment) (1:200 dilution). Cells were fixed in 4% paraformaldehyde for storage at 4° C. 10⁴ events were collected for each assay on the FAGS-Calibur (BD Immunocytometry Systems, San Jose, Calif., USA).

Only fifteen out of over 500 putative binders were identified which specifically bound to Priess cells. These clones were further analysed for immunomodulatory ability and for their killing activity as described below. Table 1 contains the sequence characteristics of clones MS-GPC-1, 2, 3, 4, 5, 6, 7, 8, 10, 11, 14, 15 & 16 identified thereby. The VH and VL families and the CDR3s listed refer to the HuCAL consensus-based antibody genes as described (Knappik et al., 2000); the sequences of the VH and VL CDRs are shown in Table 1, and the full sequences of the VH and VL domains are shown in FIG. 15.

3. Generation of Fab-Fragments.

3.1. Conversion of scFv to Fab

The Fab-fragment antigen binding polypeptides MS-GPC-1-Fab, MS-GP-6-Fab, MS-GPC-8-Fab and MS-GPC-10-Fab were generated from their corresponding scFv fragments as follows. Both heavy and light chain variable domains of scFv fragments were cloned into pMx9_Fab (FIG. 13), the heavy chain variable domains as MfeI/StyI-fragments, the variable domains of the kappa light chains as EcoRV/BsiWI-fragments. The lambda chains were first amplified from the corresponding pMORPH13_scFv vector as template with PCR-primers CRT5 (5′ primer) and CRT6 (3′ primer), wherein CRT6 introduces a unique DraIII restriction endonuclease site.

CRT5:
5′ GTGGTGGTTCCGATATC 3′
CRT6:
5′ AGCGTCACACTCGGTGCGGCTTTCGGCTGGCCAAGAACGGGTTA 3′

The PCR product is cut with EcoRV/DraIII and cloned into pMx9_Fab (see FIG. 13). The Fab light chains could be detected with a polyclonal goat anti-human IgG antibody-R-Phycoerythrin-conjugate (Jackson ImmunoResearch, West Grove, Pa., USA, Cat. No. 109-116-088, F(ab′)₂ fragment) (1:200 dilution).

3.2. Expression and Purification of HuCAL-Antibody Fragments in E. Coli

Expression in E. coli cells; (JM83) of scFv and Fab fragments from pMx7_FS or pMx9_Fab, respectively, were carried out in one litre of 2×TY-medium supplemented with 34 μg/ml chloramphenicol. After induction with 0.5 mM IPTG (scFv) or 0.1 mM IPTG (Fab), cells were grown at 22° C. for 12 hours. Cell pellets were lysed in a French Press (Thermo Spectronic, Rochester, N.Y., USA) in 20 mM sodium phosphate, 0.5 M NaCl, and 10 mM imidazole (pH 7.4). Cell debris was removed by centrifugation and the clear supernatant filtered through 0.2 μm pores before subjecting it to STREP tag purification using a Streptactin matrix and purification conditions according to the supplier (IBA GmbH, Göttingen, Germany). Purification by size exclusion chromatography (SEC) was performed as described by Rheinnecker et al. (1996). The apparent molecular weights were determined by SEC with calibration standards and confirmed in some instances by coupled liquid chromatography-mass spectrometry (TopLab GmbH, Martinsried, Germany).

4. Optimization of Antibody Fragments

In order to optimize certain biological characteristics of the HLA-DR binding antibody fragments, one of the Fab fragments, MS-GPC-8-Fab, was used to construct a library of Fab antibody fragments by replacing the parental VL λ1 chain by the pool of all lambda chains λ 1-3 randomized in CDR3 from the HuCAL library (Knappik et al., 2000).

The Fab fragment MS-GPC-8-Fab (see 3.1) was cloned via XbaI/EcoRI from pMx9_Fab_GPC-8 into pMORPH18_Fab, a phagemid-based vector for phage display of Fab fragments, to generate pMORPH18_Fab_GPC-8 (see FIG. 14). A lambda chain pool comprising a unique DraIII restriction endonuclease site (Knappik et al., 2000) was cloned into pMORPH18_Fab_GPC-8 cut with NsiI and DraIII (see vector map of pMORPH18_Fab_GPC-8 in FIG. 14).

The resulting Fab optimization library was screened by two rounds of panning against MHC-class II DRA*0101/DRB1*0401 (prepared as above) as described in 2.3 with the exception that in the second round the antigen concentration for coating was decreased to 12 ng/well). FACS identified optimized clones as described above in 2.5. Six of these clones, MS-GPC-8-1, MS-GPC-8-6, MS-GPC-8-9, MS-GPC-8-10, MS-GPC-8-17, MS-GPC-8-18 and MS-GPC-8-27, were further characterized and showed cell killing activity as found for the starting fragment MS-GPC-8. Table 1 contains the sequence characteristics of MS-GPC-8-1, MS-GPC-8-6, MS-GPC-8-9, MS-GPC-8-10, MS-GPC-8-17, MS-GPC-8-18 and MS-GPC-8-27. The VH and VL families and the CDR3s listed refer to the HuCAL consensus-based antibody genes as described (Knappik et al., 2000), the full sequences of the VH and VL domains of MS-GPC-8-6, MS-GPC-8-10, MS-GPC-8-17 and MS-GPC-8-27are shown in FIG. 15.

The optimized Fab forms of the anti-HLA-DR antibody fragments MS-GPC-8-6 and MS-GPC-8-17 showed improved characteristics over the starting MS-GPC-8. For example, the EC50 of the optimized antibodies was 15-20 and 5-20 nM (compared to 20-40 nM for MS-GPC-8, where the concentration is given as the concentration of the bivalent cross-linked Fab dimer), and the maximum capacity to kill MHH-Call 4 cells determined as 76 and 78% for MS-GPC-8-6 and MS-GPC-8-17 (compared to 65% for MS-GPC-8) respectively.

For further optimization, the VL CDR1 regions of a set of anti-HLA-DR antibody fragments derived from MS-GPC-8 (including MS-GPC-8-10 and MS-GPC-8-27) were optimized by cassette mutagenesis using trinucleotide-directed mutagenesis (Virnekäs et al., 1994). In brief, a VI1 CDR1 library cassette was synthesized containing six randomized positions (total variability: 7.43×10⁶), and was cloned into a VI1 framework. The CDR1 library was digested with EcoRV and BbsI, and the fragment comprising the CDR1 library ligated into the lambda light chains of the MS-GPC-8-derived Fab antibody fragments in pMORPH18_Fab (as described above), Digested with EcoRV and BbsI. The resulting library was screened as described above. Ten clones were identified as above by binding specifically to HLA DR (MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 & MS-GPC-8-27-41) and showed cell killing activity as found for the starting fragments MS-GPC-8, MS-GPC-8-10 and MS-GPC-8-27. Table 1 contains the sequence characteristics of MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 & MS-GPC-8-27-41. The VH and VL families and the CDR3s listed refer to the HuCAL consensus-based antibody genes as described (Knappik et al., 2000), the full sequences of the VH and VL domains of MS-GPC-8-6-13, MS-GPC,8-10-57 & MS-GPC-8-27-41 are shown in FIG. 15.

From these 10 clones, four Fab fragments were chosen (MS-GPC-8-6-2, MS-GPC-8-6-13, MS-GPC-8-10-57 and MS-GPC-8-27-41) as demonstrating significantly improved EC50 of cell killing as described in example 10. Table 1 shows the sequences of clones optimised at the CDR1 region.

Optimisation procedures not only increased the biological efficacy of anti-HLA DR antibody fragments generated by the optimisation process, but a physical characteristic—affinity of the antibody fragment to HLA DR protein—was also substantially improved. For example, the affinity of Fab forms of MS-GPC-8 and its optimised descendents was measured using a surface plasmon resonance instrument (Biacore, Upsala Sweden) according to example 7. The affinity of the MS-GPC-8 parental Fab was improved over 100 fold from 346 nM to ˜60 nM after VLCDR3 optimisation and further improved to single digit nanomolar affinity (range 3-9 nM) after VLCDR3+1 optimisation (Table 2).

5. Generation of IgG

5.1 Construction of HuCAL-Immunoglobulin Expression Vectors

Heavy chains were cloned as follows. The multiple cloning site of pcDNA3.1+(Invitrogen) was removed (NheI/ApaI), and a stuffer compatible with the restriction sites used for HuCAL-design was inserted for the ligation of the leader sequences (NheI/EcoRI), VH-domains (EcoRI/BlpI) and the immunoglobulin constant regions (BlpI/ApaI). The leader sequence (EMBL M83133) was equipped with a Kozak sequence (Kozak, 1987). The constant regions of human IgG1 (PIR J00228), IgG4 (EMBL K01316) and serum IgA1 (EMBL J00220) were dissected into overlapping oligonucleotides with lengths of about 70 bases. Silent mutations were introduced to remove restriction sites non-compatible with the HuCAL-design. The oligonucleotides were spliced by overlap extension-PCR.

Light chains were cloned as follows. The multiple cloning site of pcDNA3.1/Zeo+(Invitrogen) was replaced by two different stuffers. The κ-stuffer provided restriction sites for insertion of a κ-leader (NheI/EcoRV), HuCAL-scFv Vκ-domains (EcoRV/BsiWI) and the κ-chain constant region (BsiWI/ApaI). The corresponding restriction sites in the λ-stuffer were NheI/EcoRV (λ-leader), EcoRV/HpaI (Vλ-domains) and HpaI/ApaI (λ-chain constant region). The κ-leader (EMBL Z00022) as well as the λ-leader (EMBL L27692) were both equipped with Kozak sequences. The constant regions of the human κ-(EMBL J00241) and λ-chain (EMBL M18645) were assembled by overlap extension-PCR as described above.

5.2 Generation of IgG-Expressing CHO-Cells

All cells were maintained at 37° C. in a humidified atmosphere with 5% CO2 in media recommended by the supplier. CHO-K1 (CRL-9618) were from ATCC and were co-transfected with an equimolar mixture of IgG heavy and light chain expression vectors. Double-resistant transfectants were selected with 600 μg/ml G418 and 300 μg/ml Zeocin (Invitrogen) followed by limiting dilution. The supernatant of single clones was assessed for IgG expression by capture-ELISA. Positive clones were expanded in RPMI-1640 medium supplemented with 10% ultra-low IgG-FCS (Life Technologies). After adjusting the pH of the supernatant to 8.0 and sterile filtration, the solution was subjected to standard protein A column chromatography (Poros 20A, PE Biosystems).

The IgG forms of anti-HLA-DR antigen binding domains show improved characteristics over the antibody fragments. These improved characteristics include affinity (Example 7) and killing efficiency (Examples 9, 10 and 14).

6. HLA-DR Specificity Assay and Epitope Mapping

To demonstrate that antigen-binding domains selected from the HuCAL library bound specifically to a binding site on the N-terminal domain of human MHCII receptor largely conserved between alleles and hitherto unknown in the context of cell killing by receptor cross linking, we undertook an assessment of their binding specificity, and it was attempted to characterise the binding epitope.

The Fab antibody fragments MS-GPC-8-27-7, MS-GPC-8-27-10, MS-GPC-8-6-13, MS-GPC-8-27-41, MS-GPC-8-6-47, MS-GPC-8-10-57, MS-GPC-8-6-27, MS-GPC-8 and MS-GPC-8-6 showed specificity of binding to HLA-DR protein but not to non-HLA-DR proteins. Fab fragments selected from the HuCAL library were tested for reactivity with the following antigens: HLA-DR protein (DRA*0101/DRB1*0401; prepared as example 1, and a set of unrelated non-HLA-DR proteins consisting of BSA, testosterone-BSA, lysozyme and human apotransferrin. An empty well (Plastic) was used as negative control. Coating of the antigen MHCII was performed over night at 1 μg/well in PBS (Nunc-MaxiSorp TM) whereas for the other antigens (BSA, Testosterone-BSA, Lysozyme, Apotransferrin) 10 μg/well was used. Next day wells were blocked in 5% non-fat milk for 1 hr followed by incubation of the respective antibodies (anti-MHCII-Fabs and an unrelated Fab (Mac1-8A)) at 100 ng/well for 1 h. After washing in PBS the anti-human IgG F(ab′)2-peroxidase-conjugate at a 1:10000 dilution in TBS (supplemented with 5% w/v non-fat dry-milk/0.05% v/v Tween 20) was added to each well for 1 h. Final washes were carried out in PBS followed the addition the substrate POD (Roche). Color-development was read at 370 nM in an ELISA-Reader.

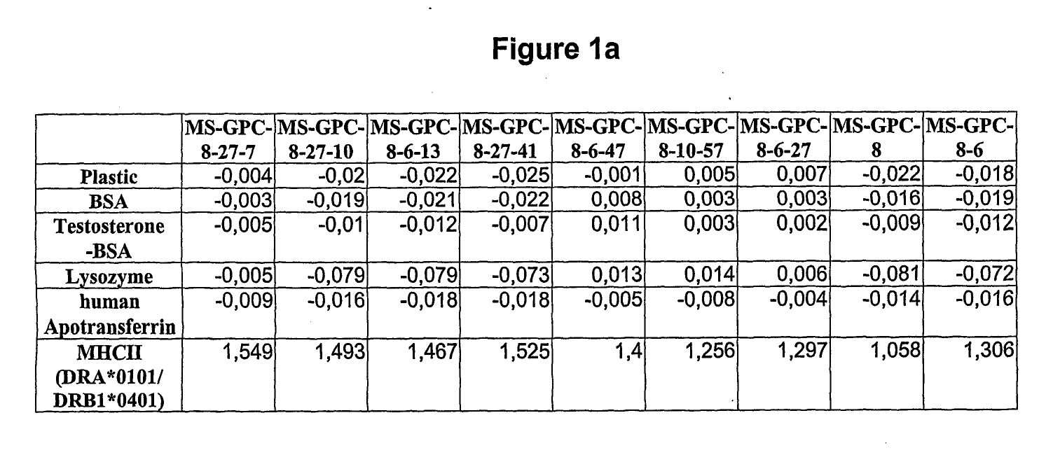

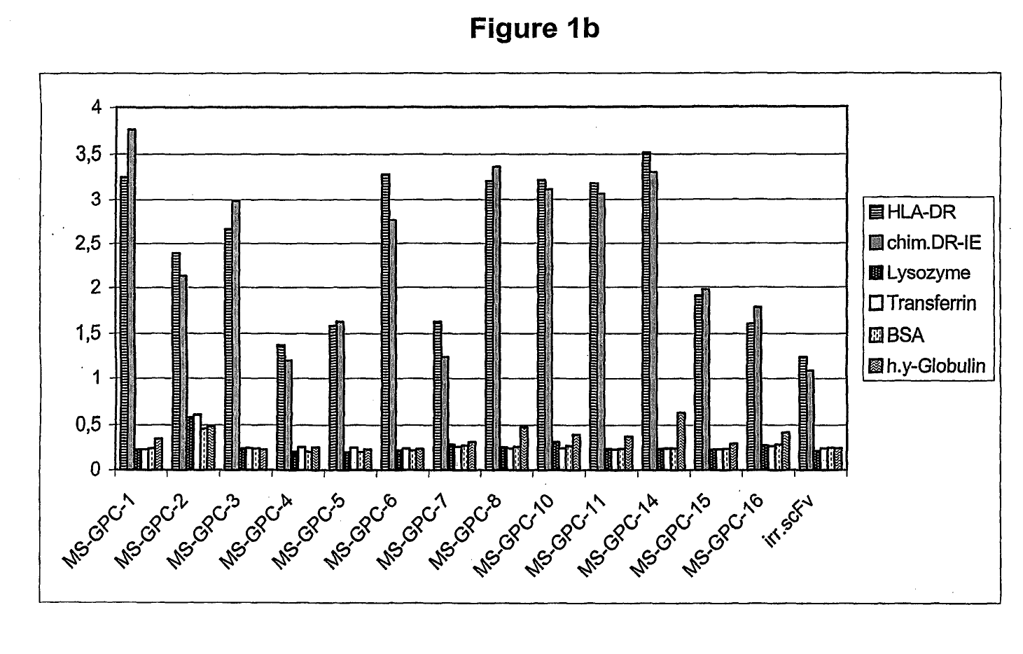

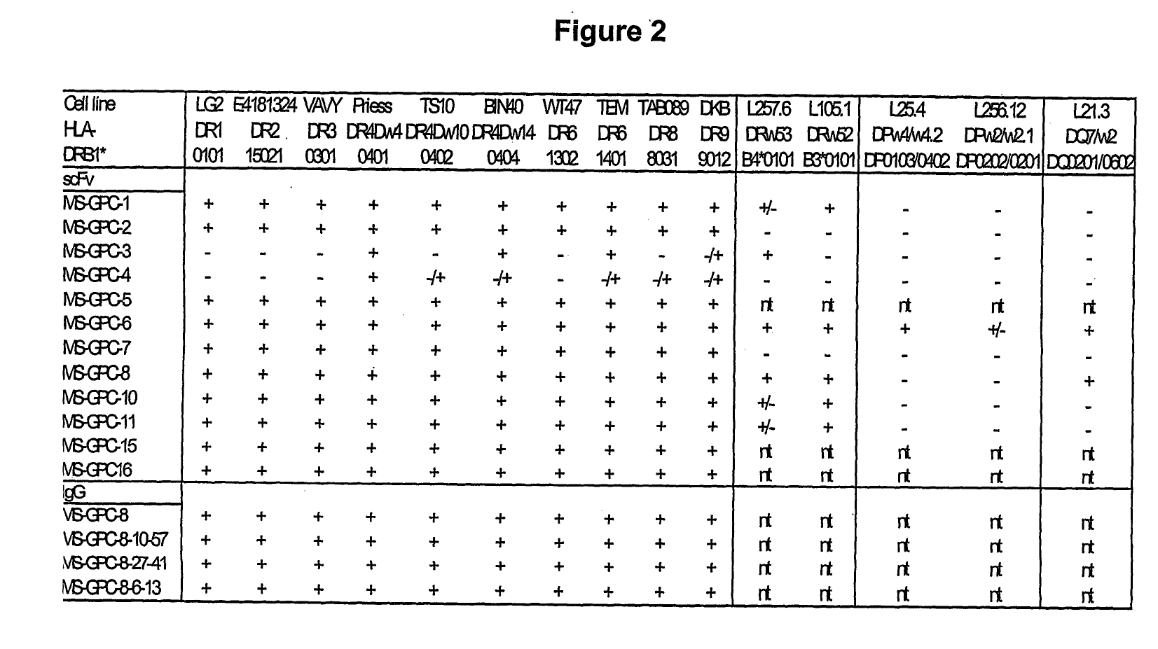

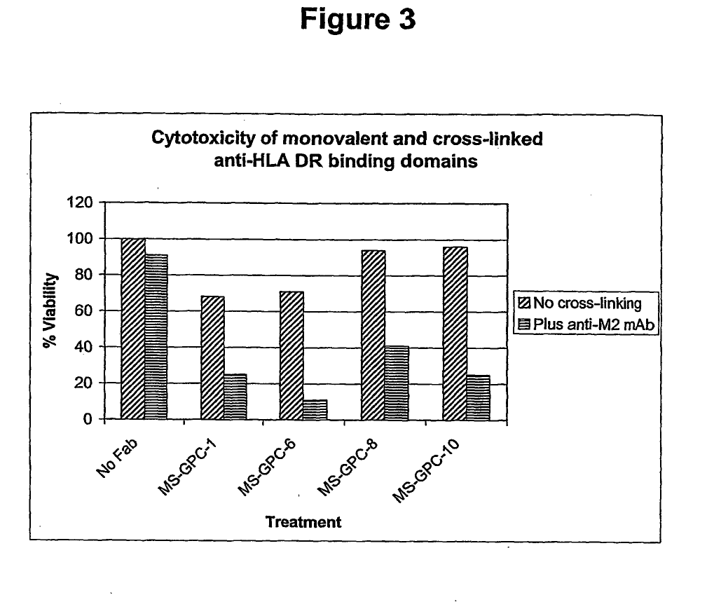

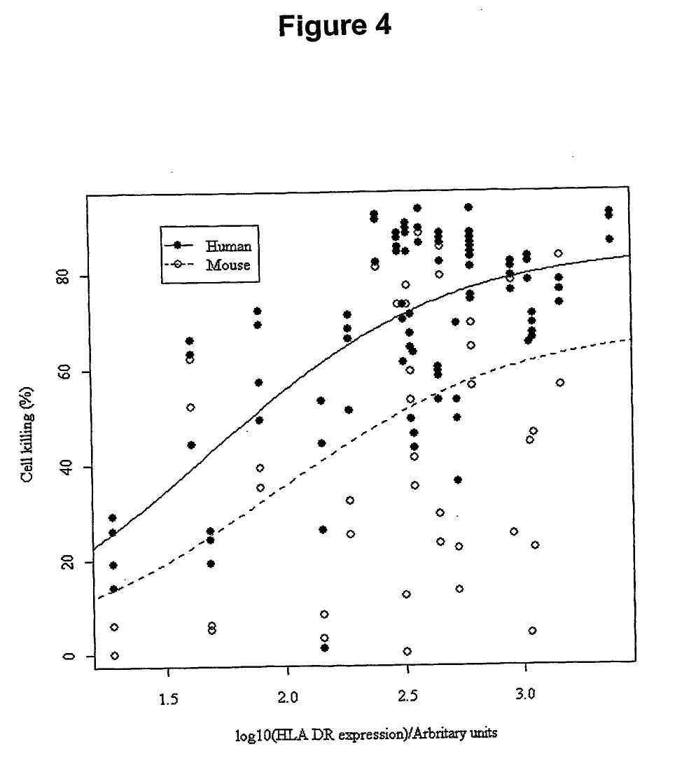

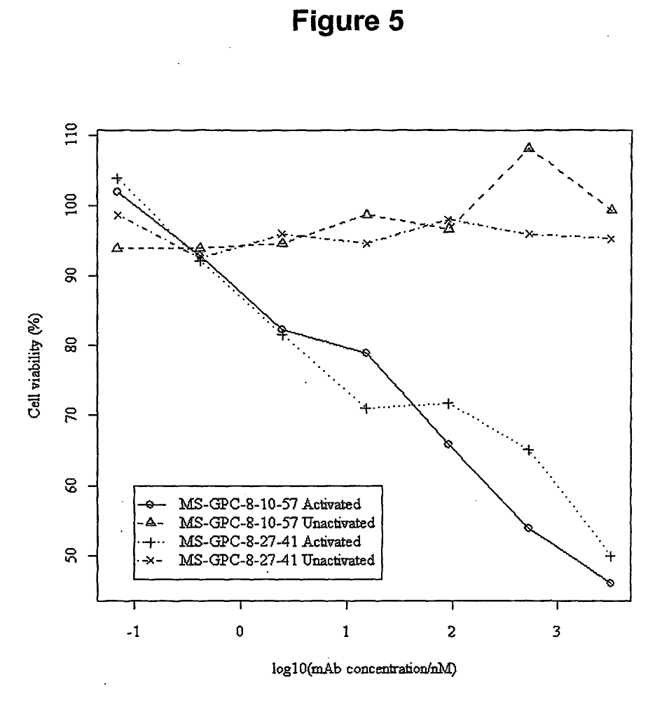

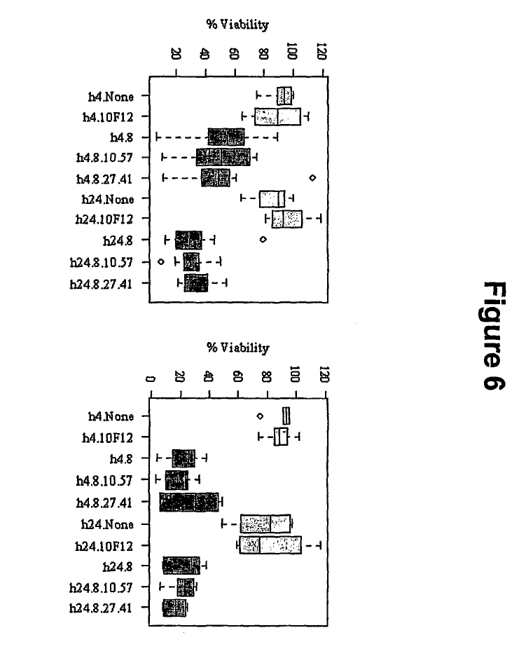

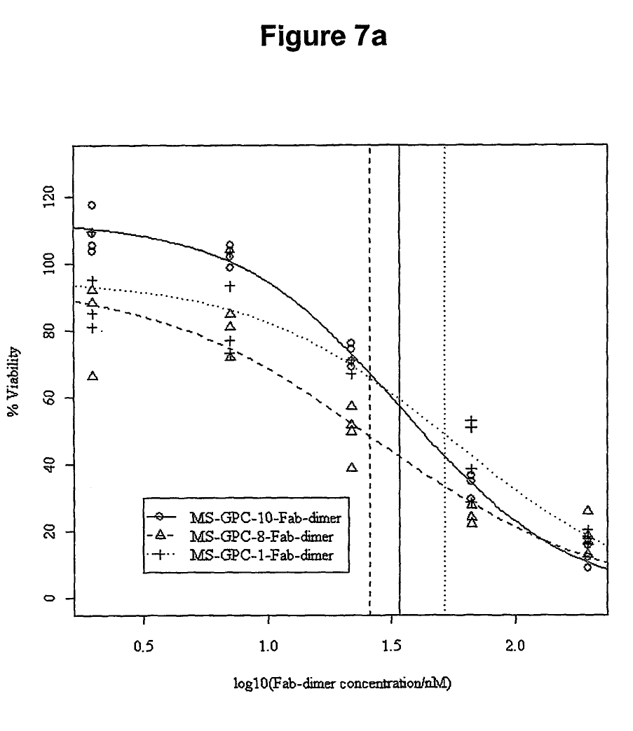

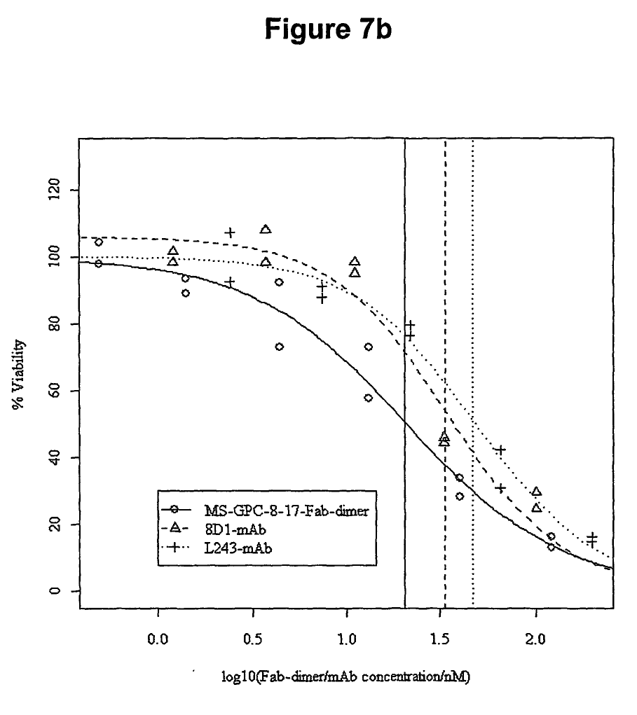

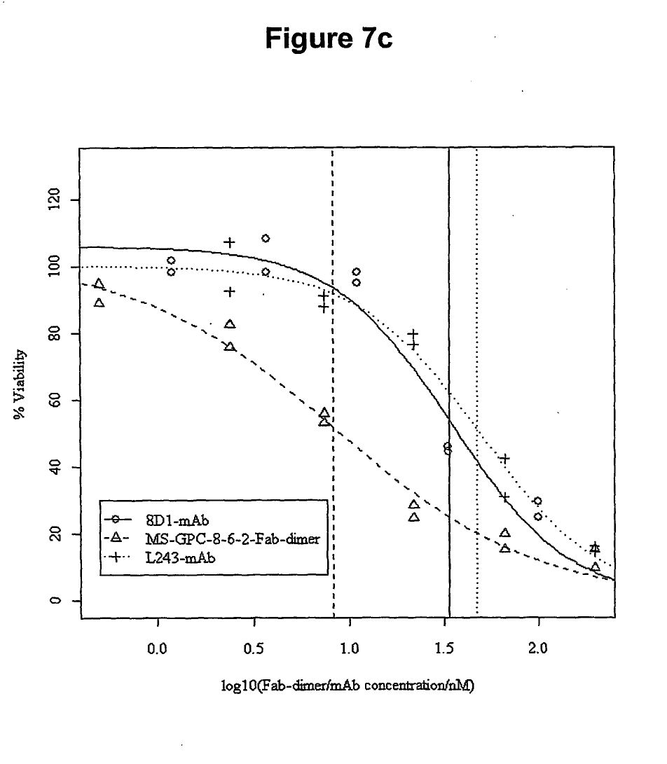

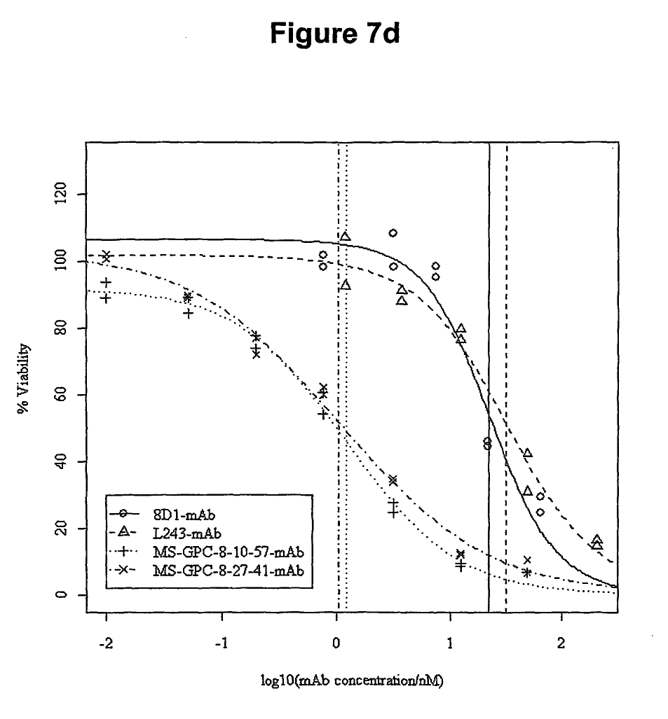

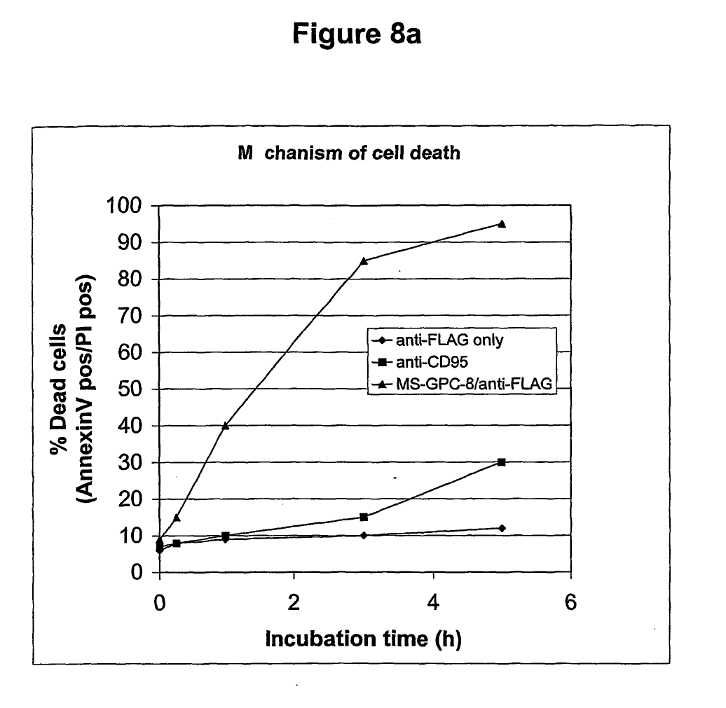

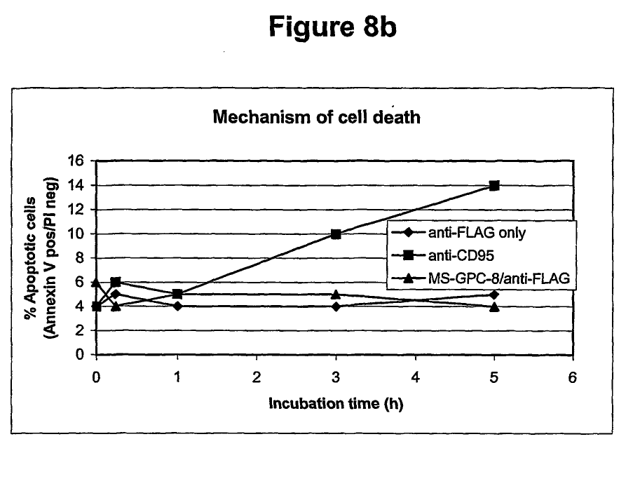

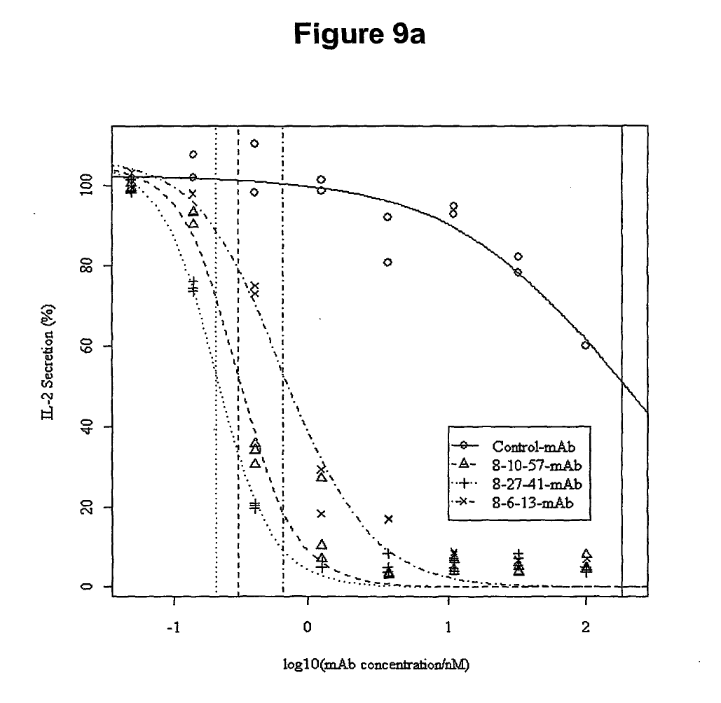

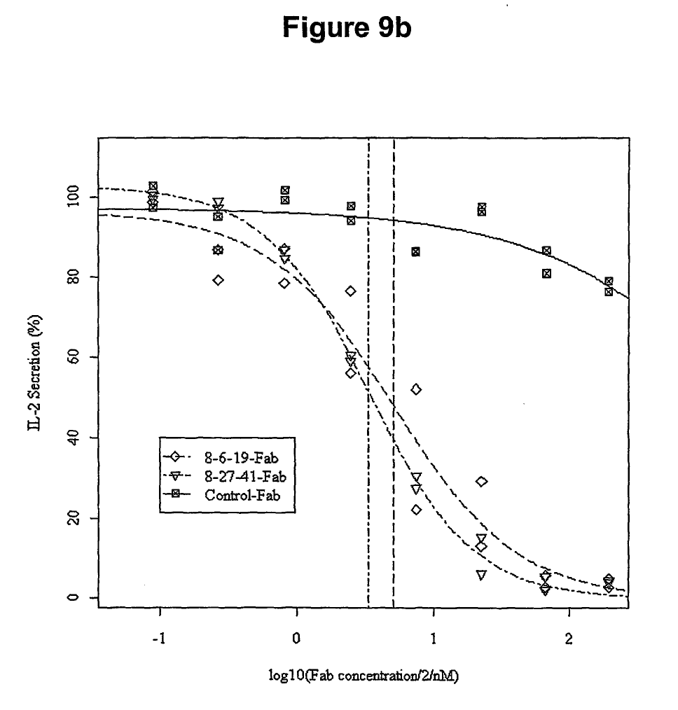

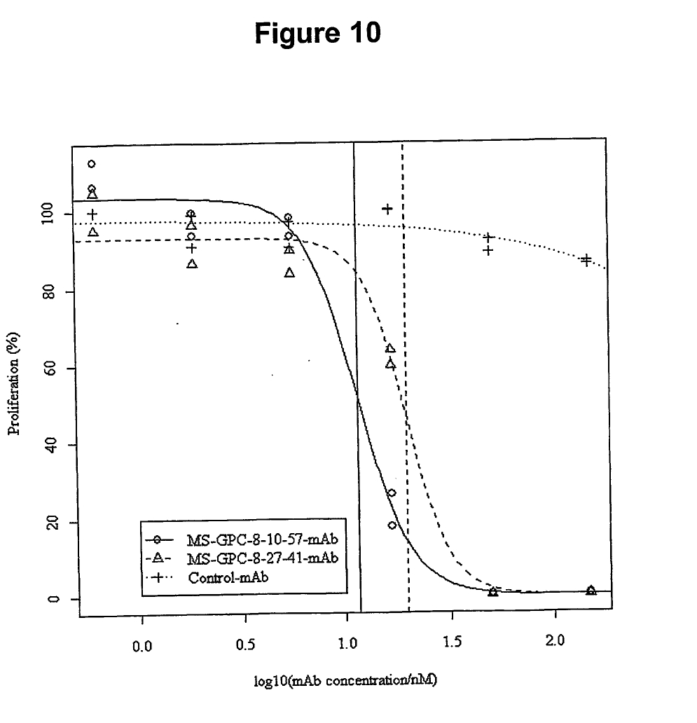

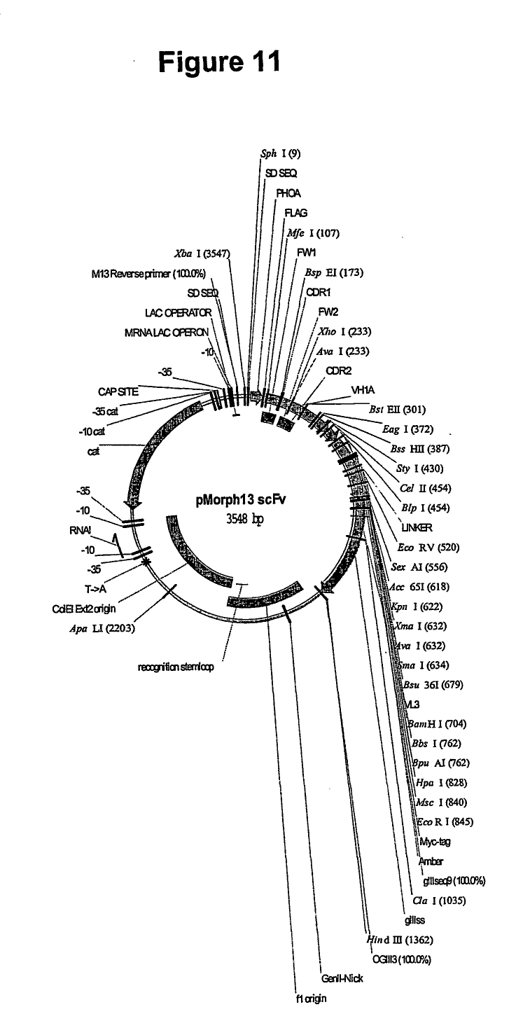

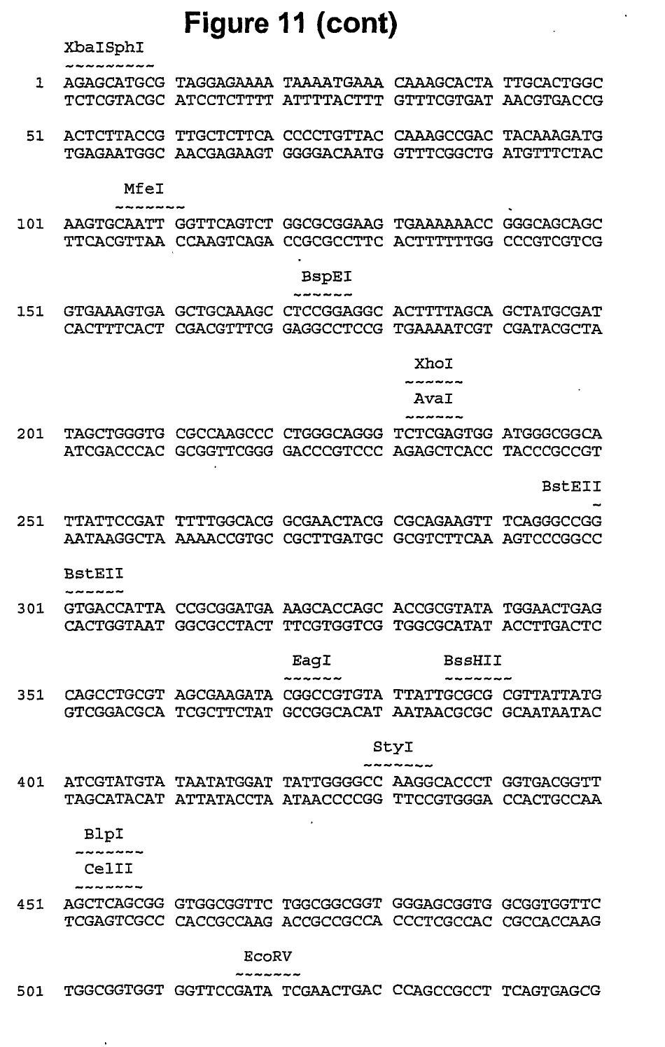

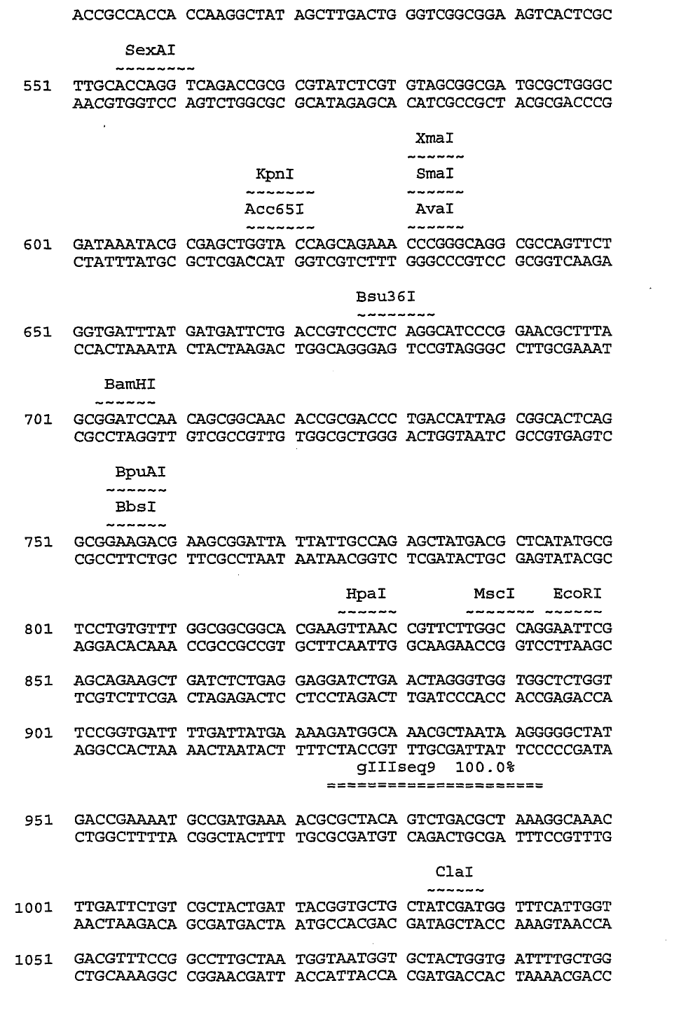

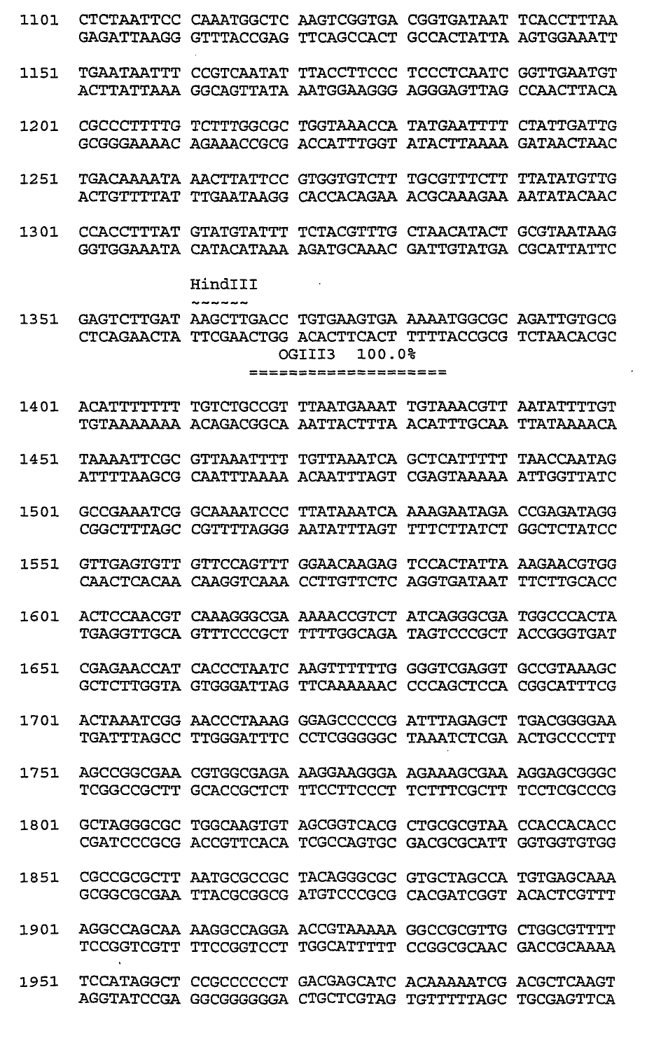

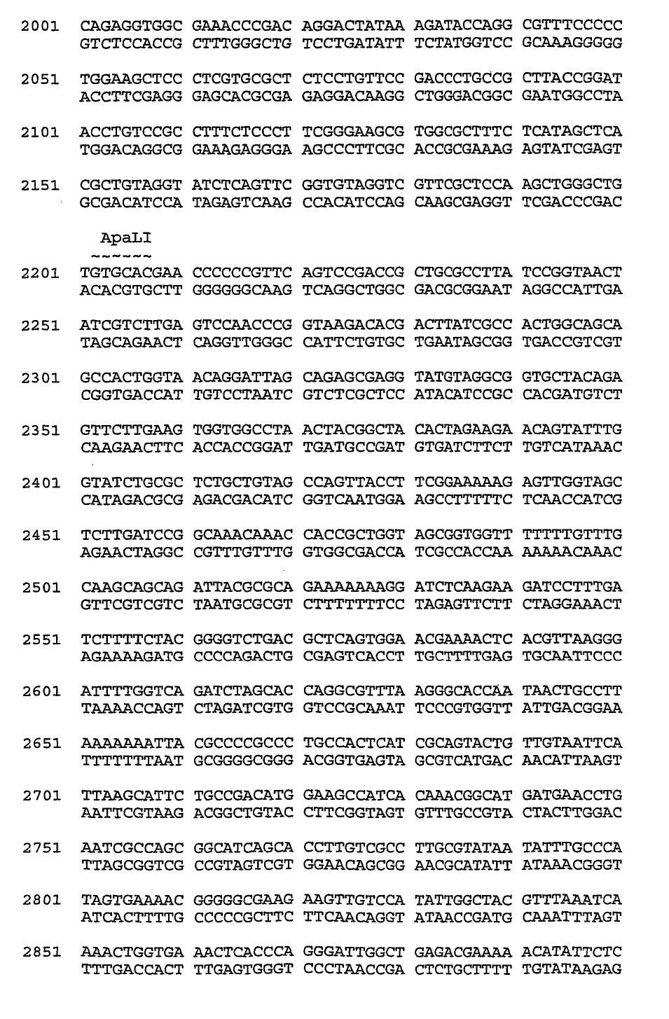

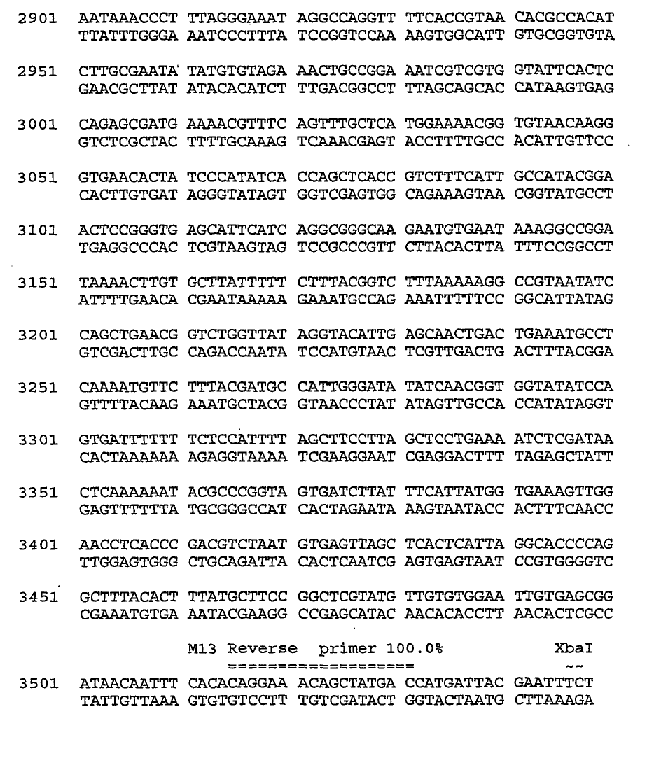

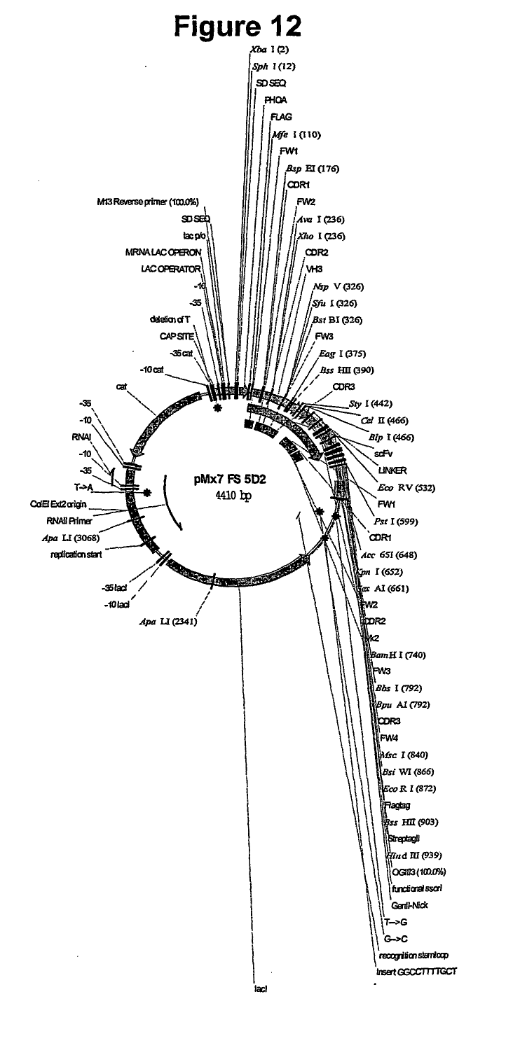

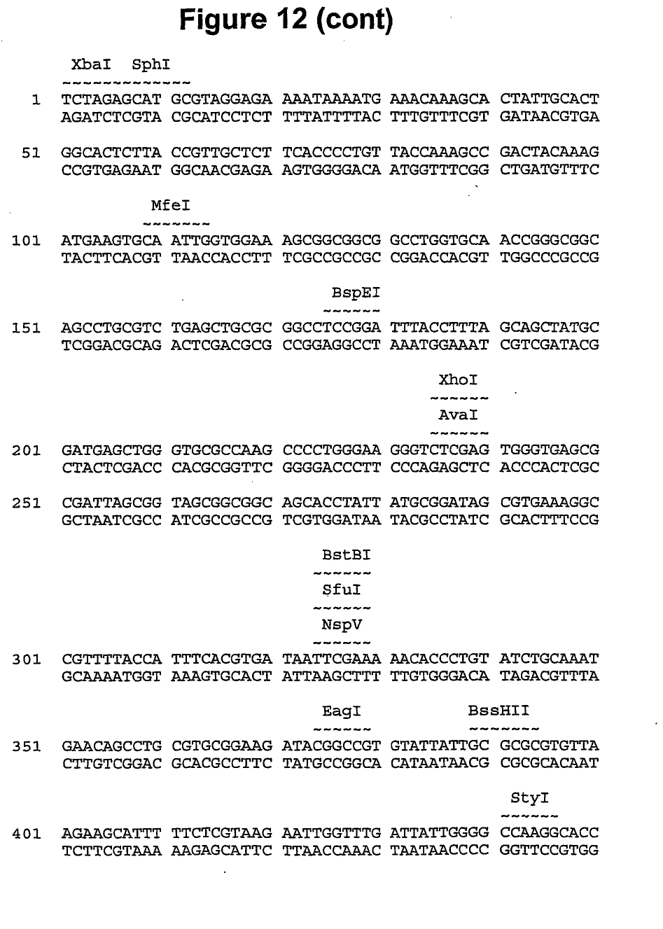

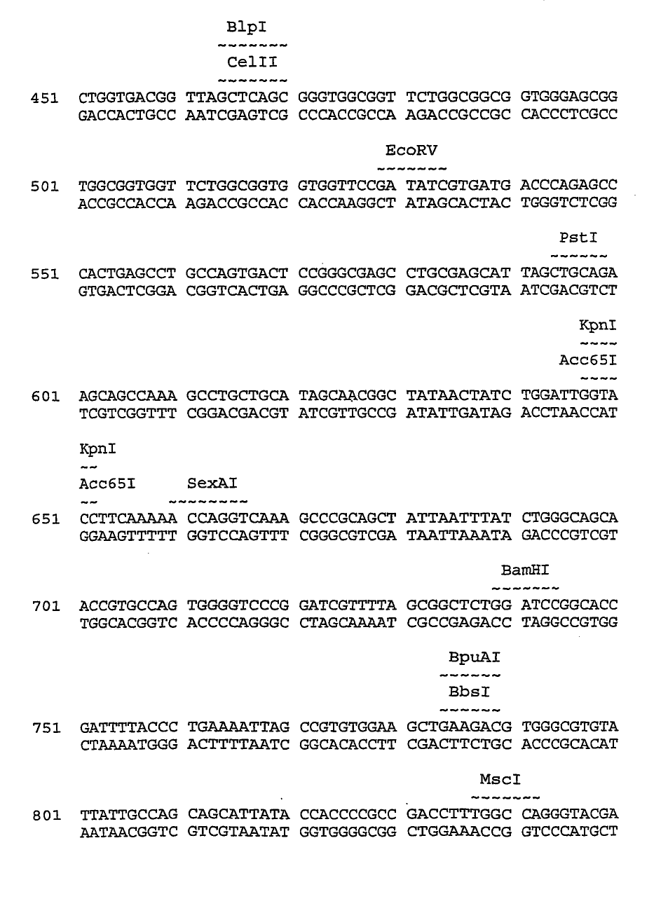

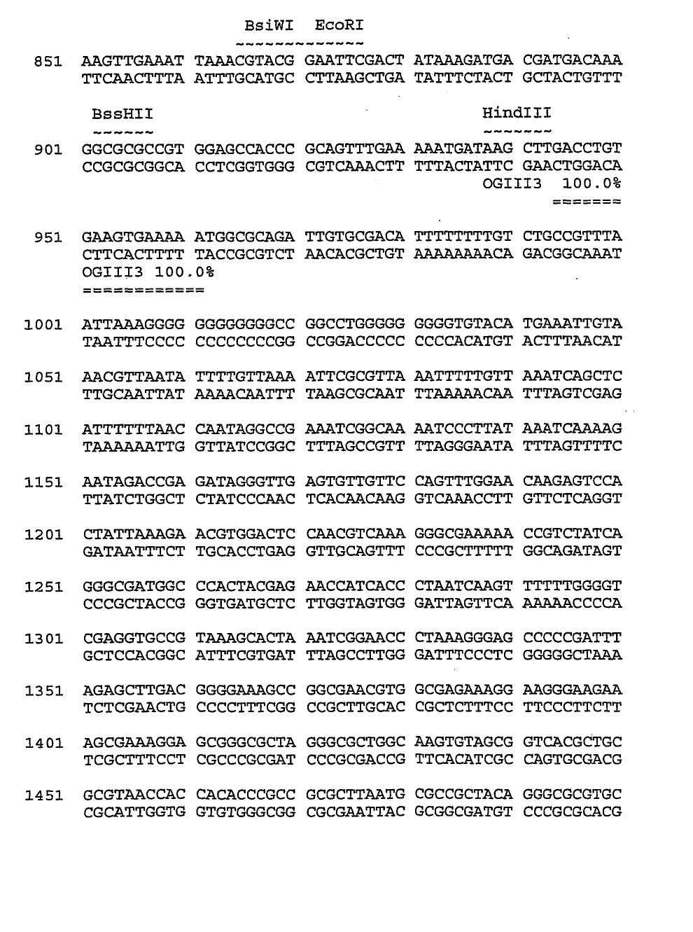

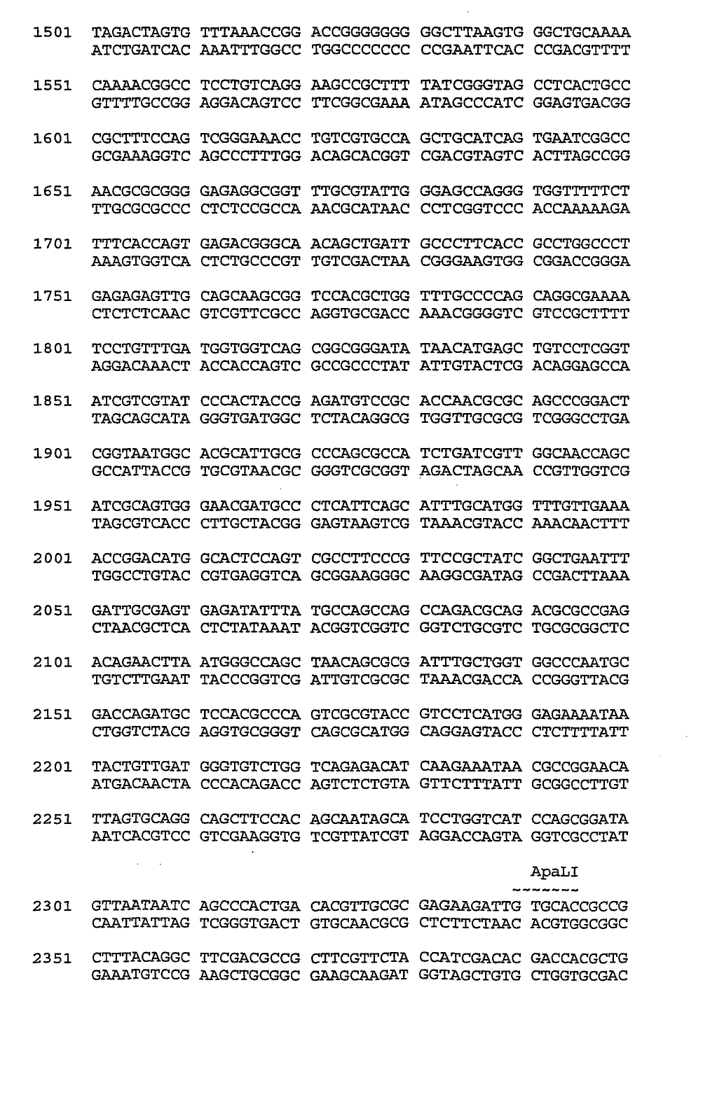

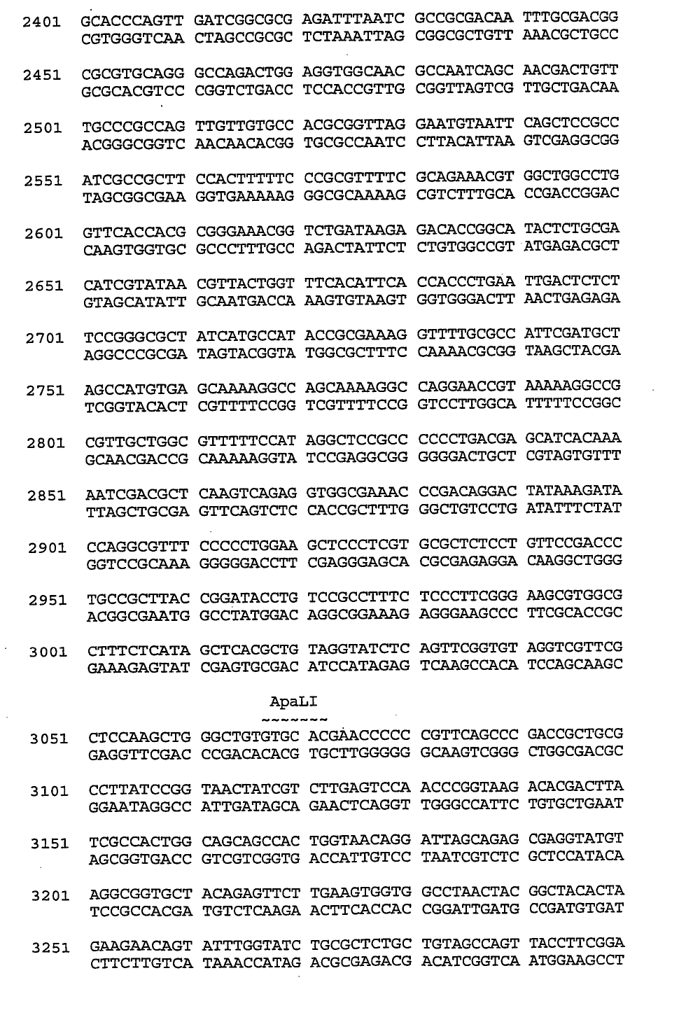

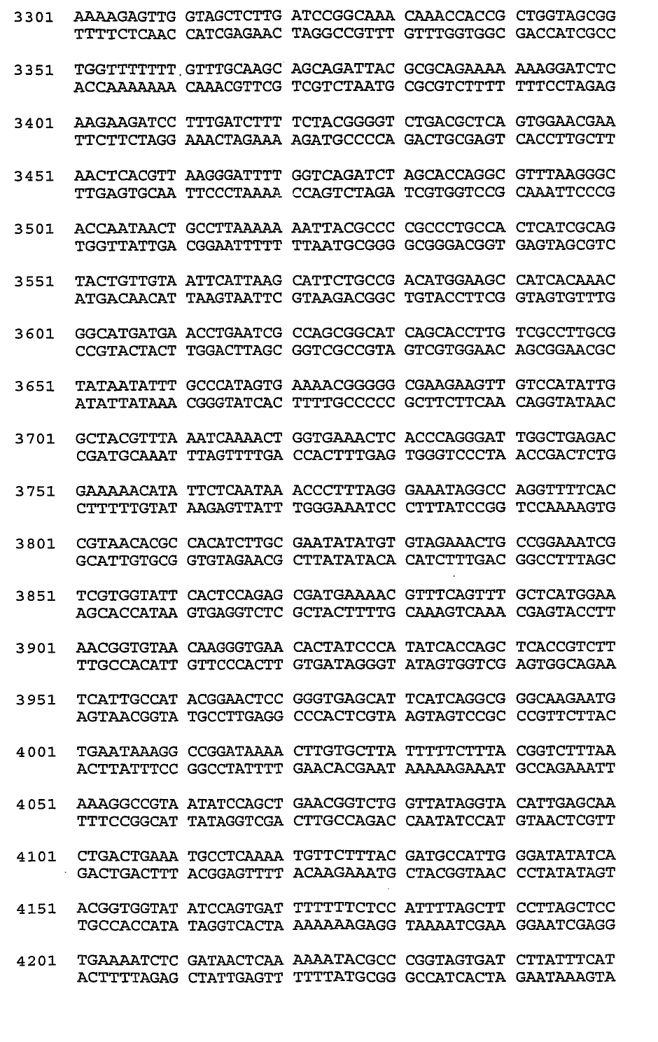

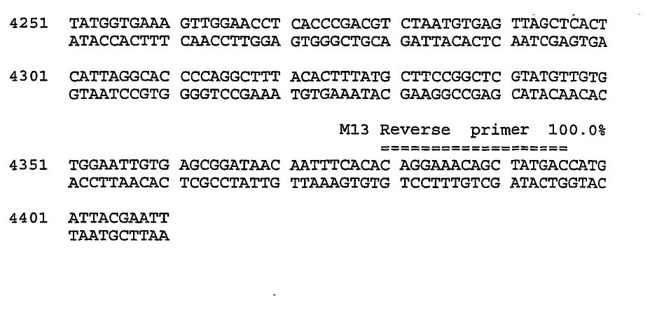

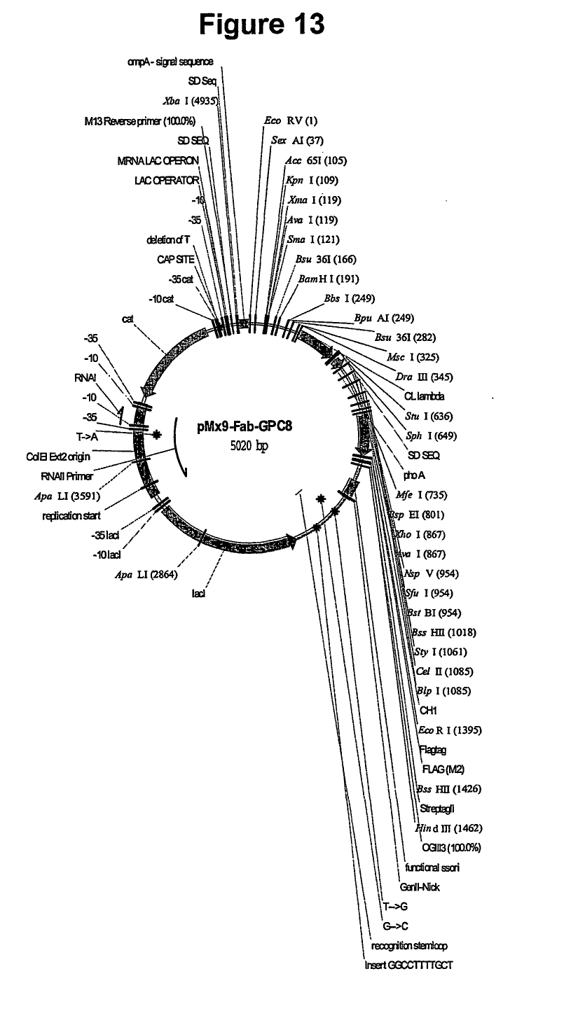

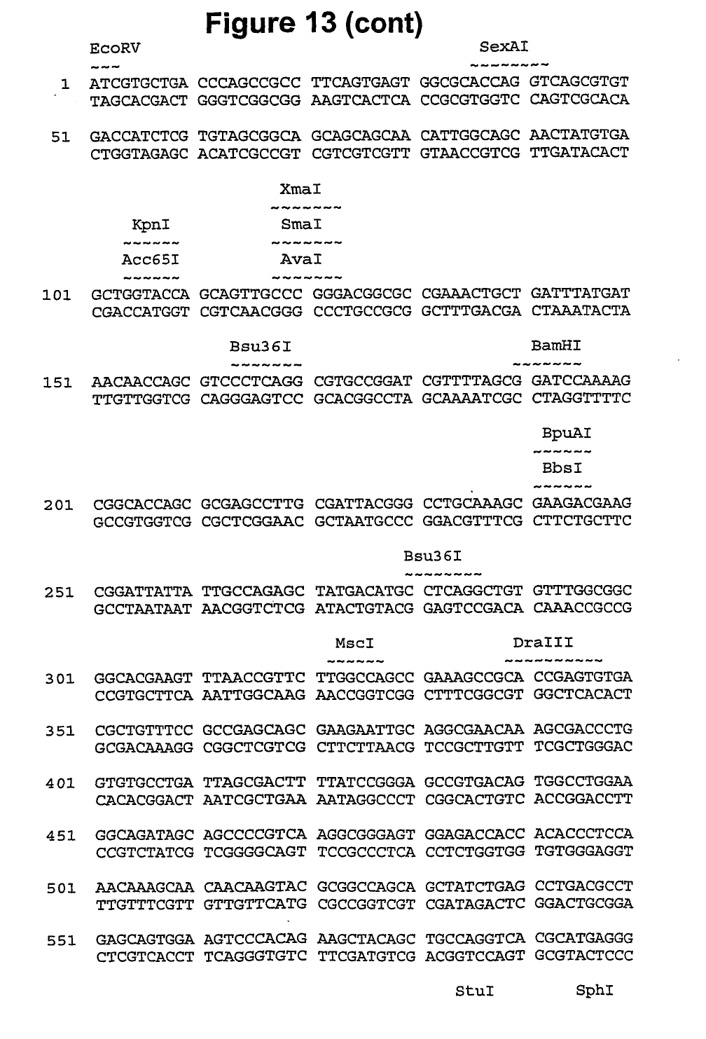

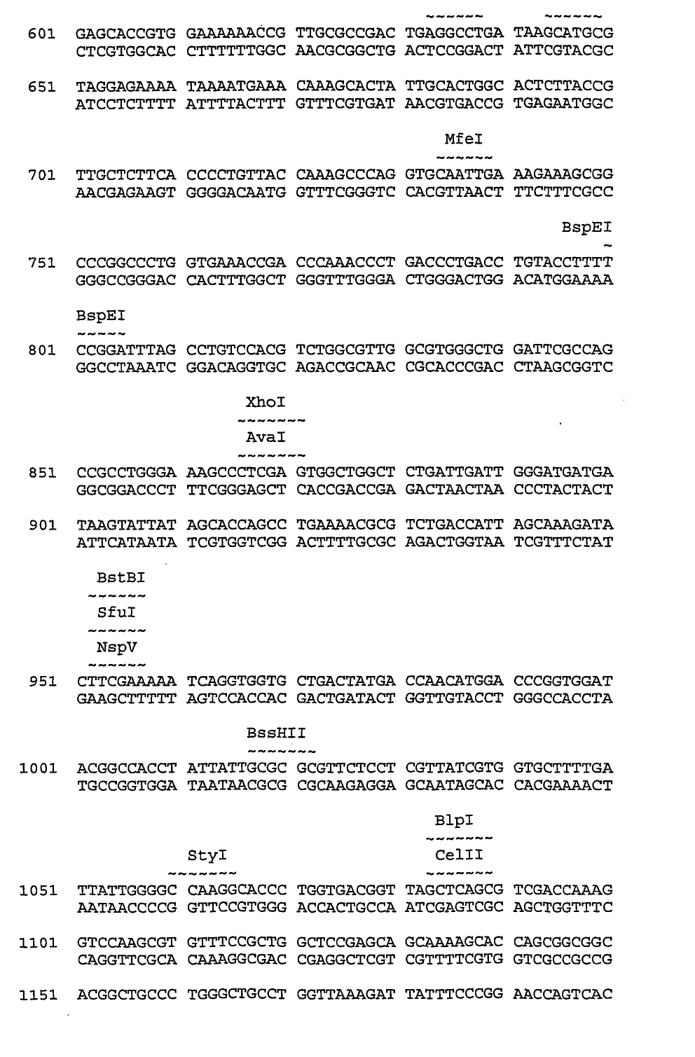

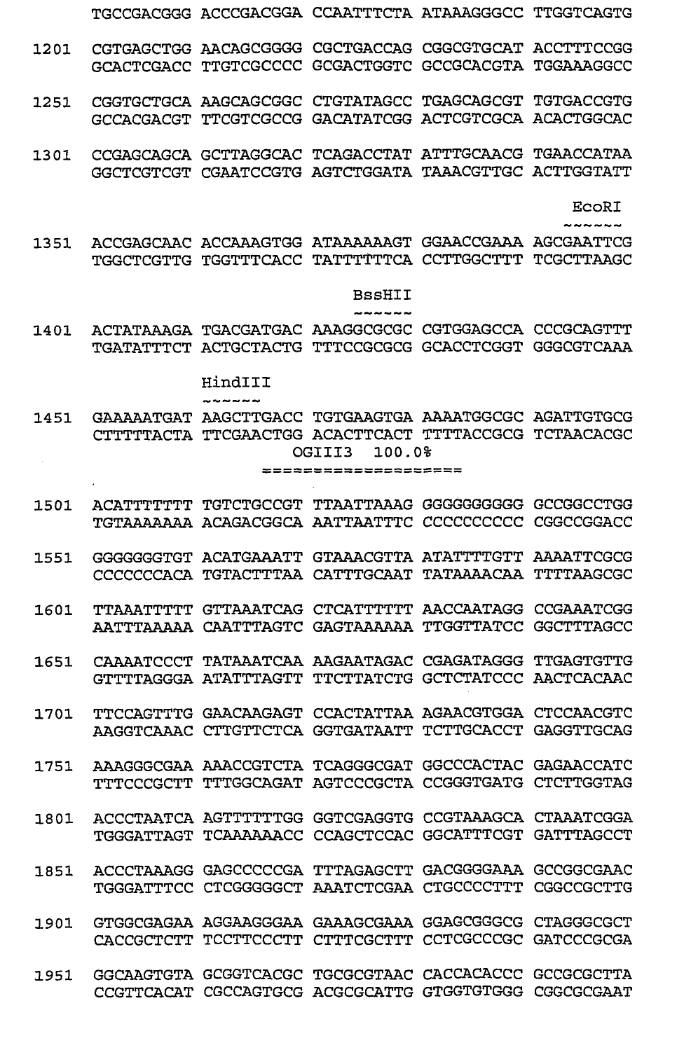

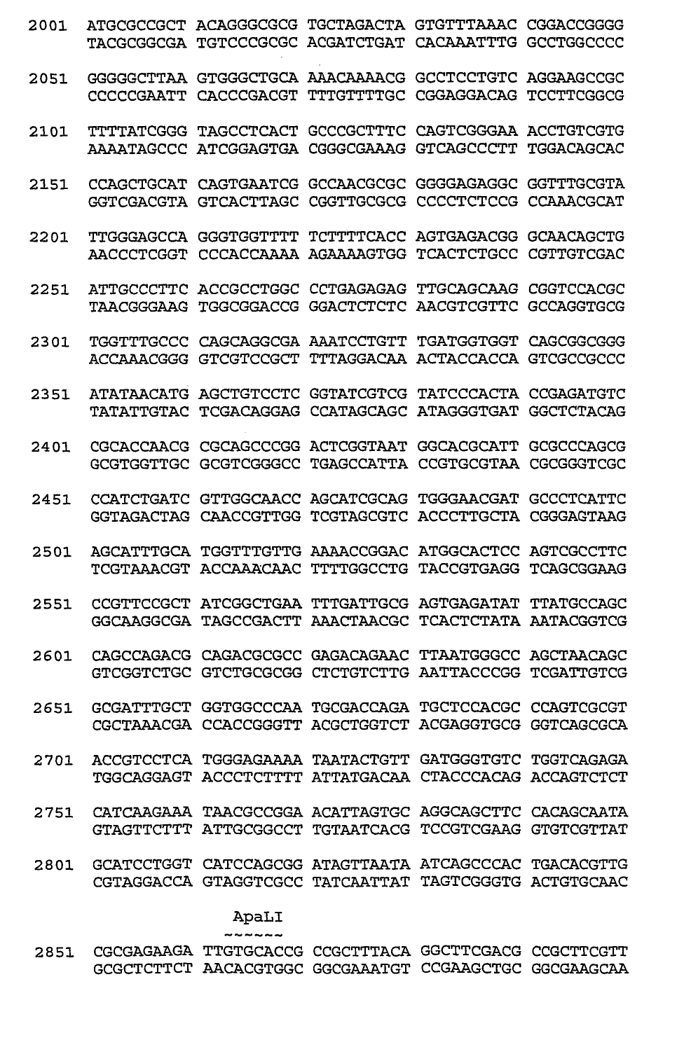

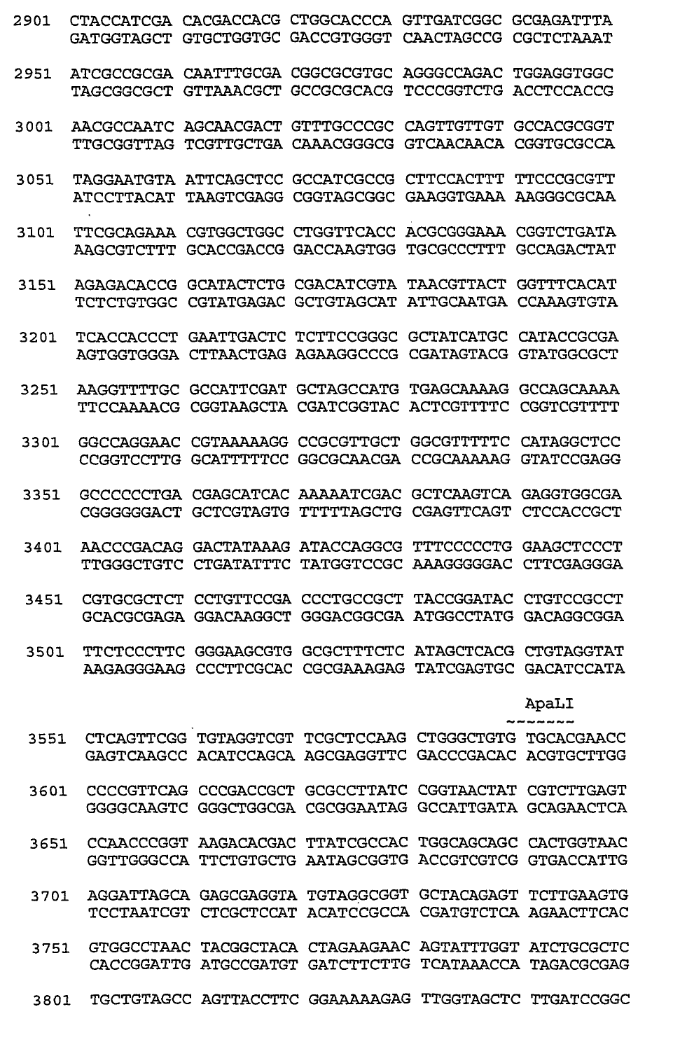

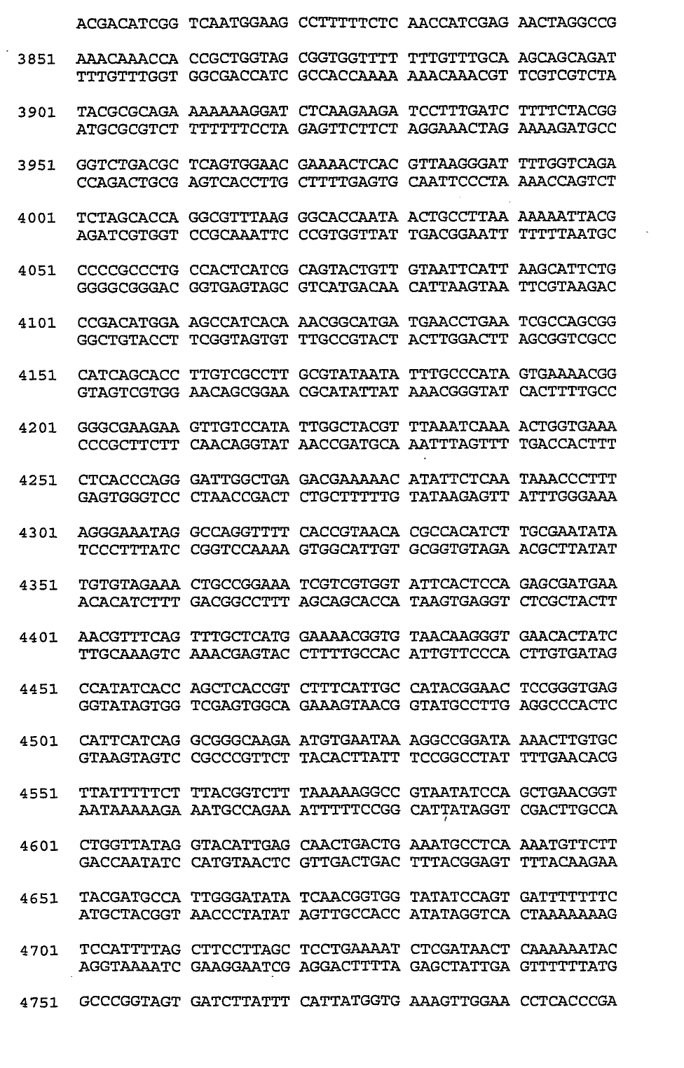

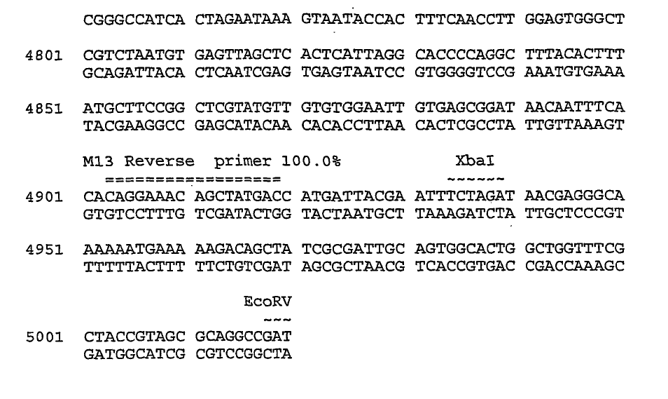

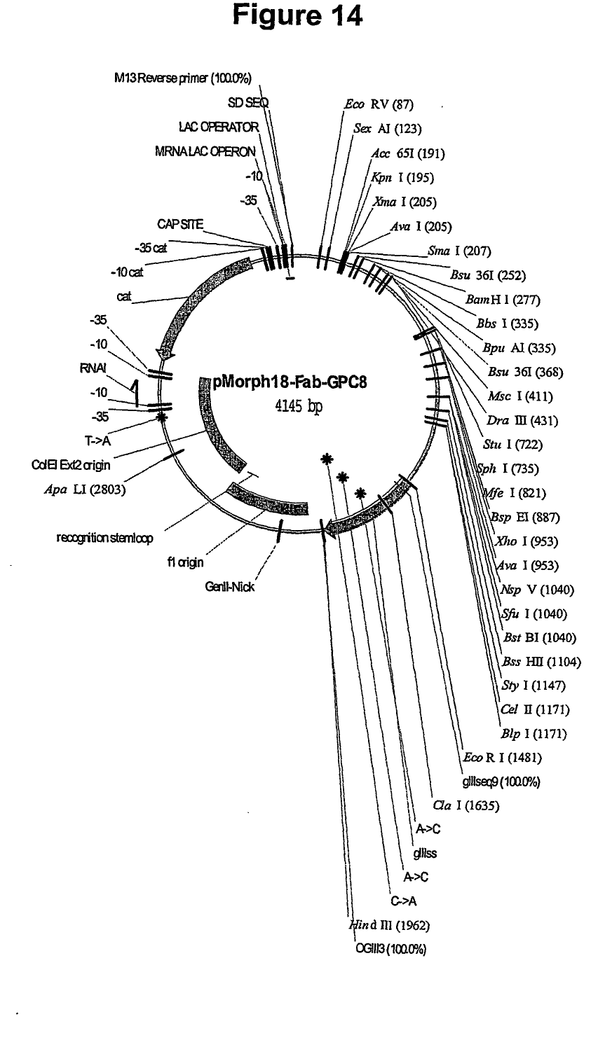

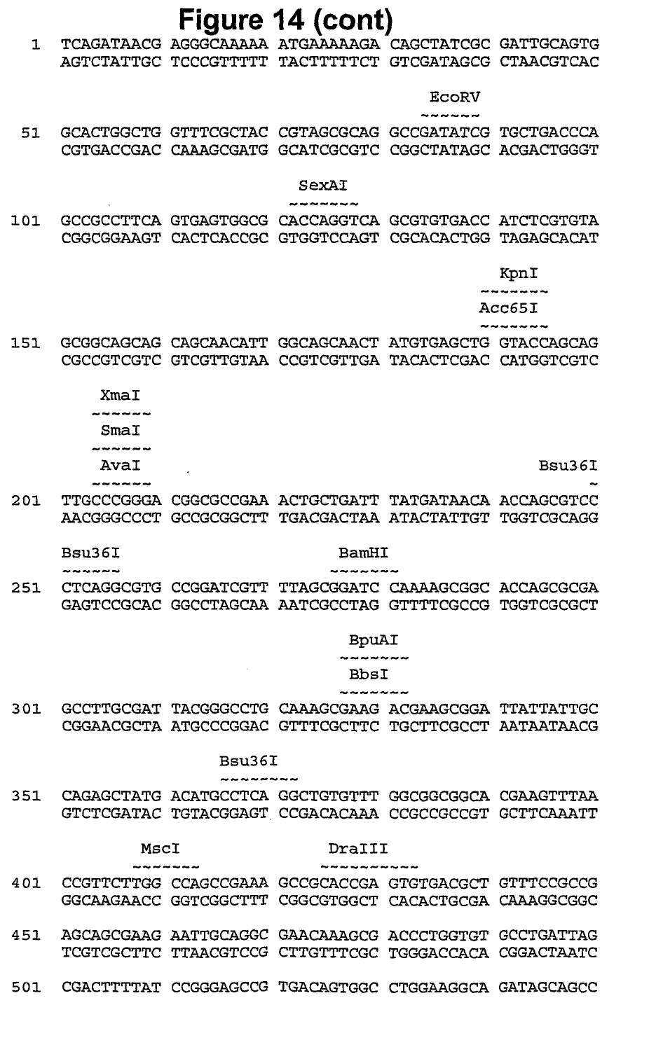

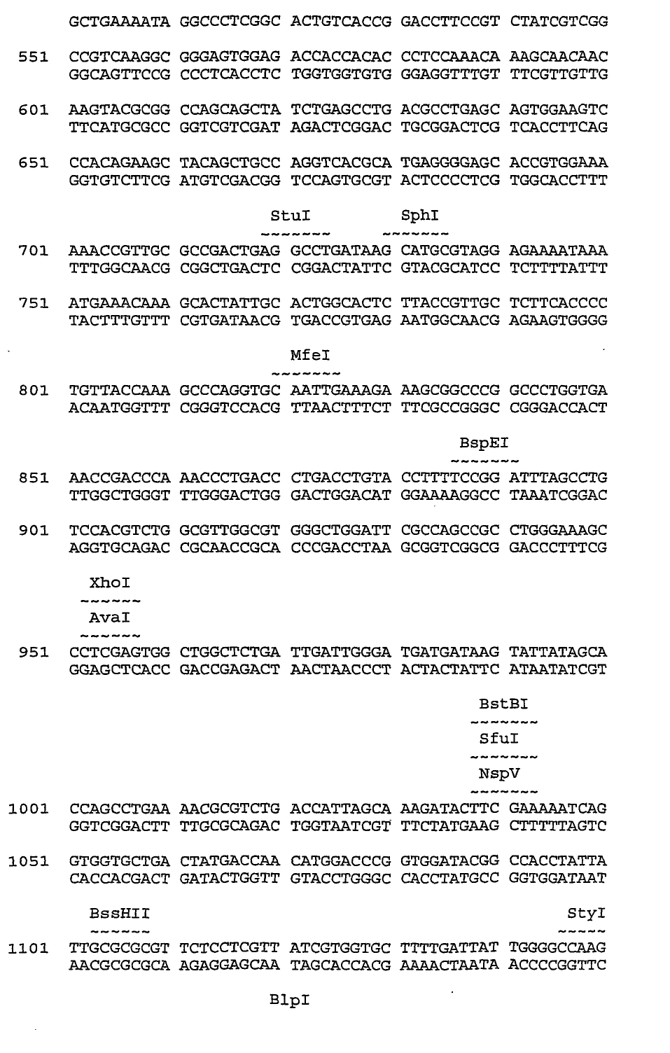

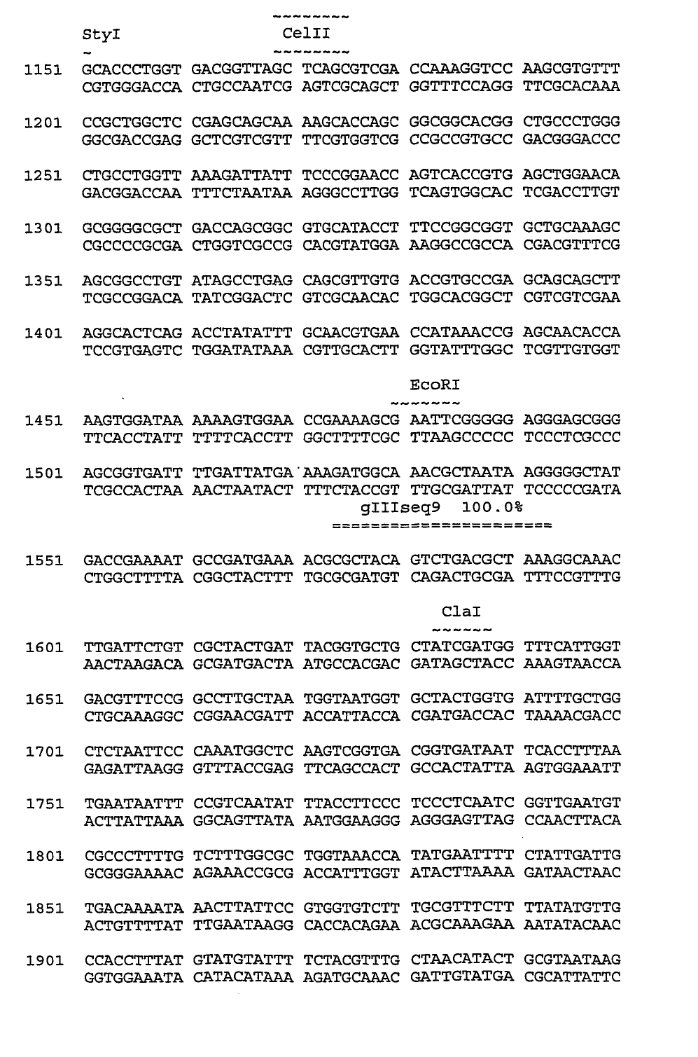

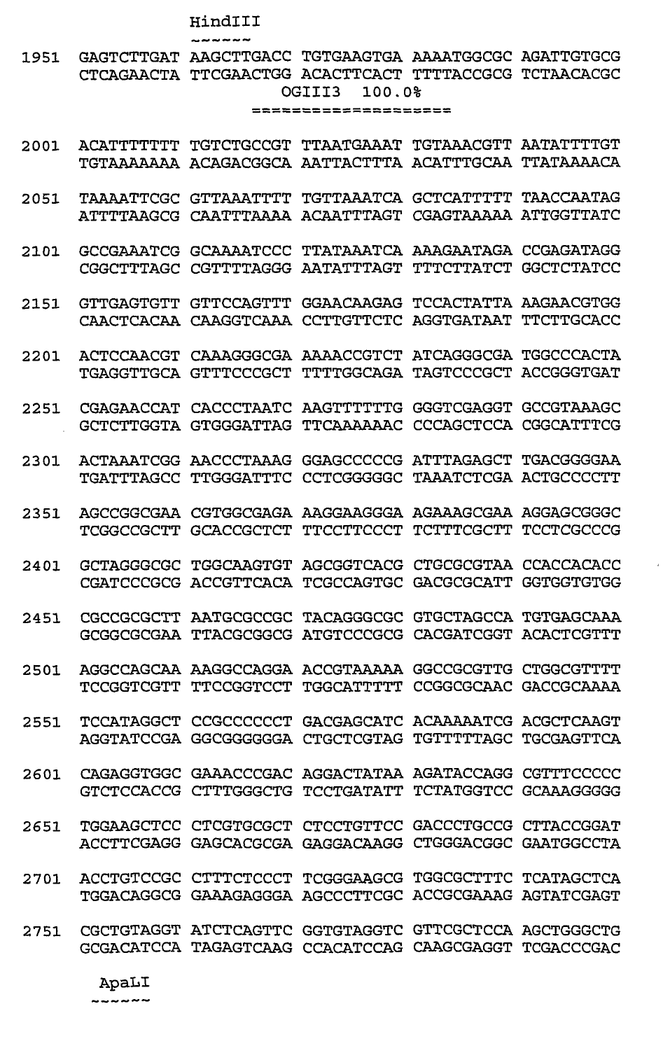

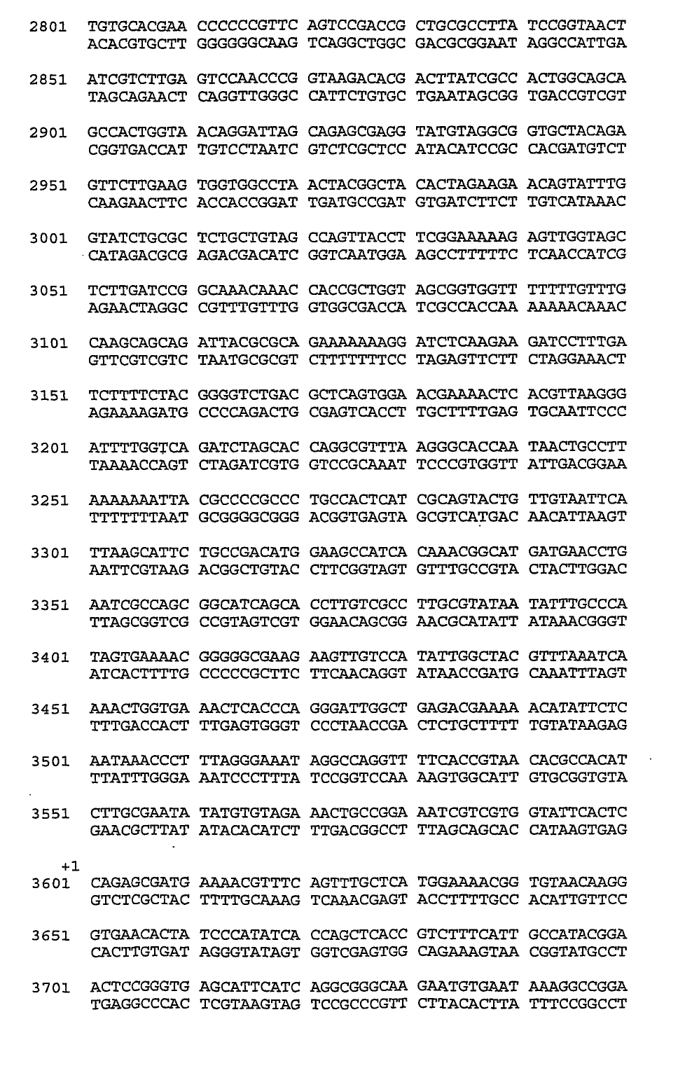

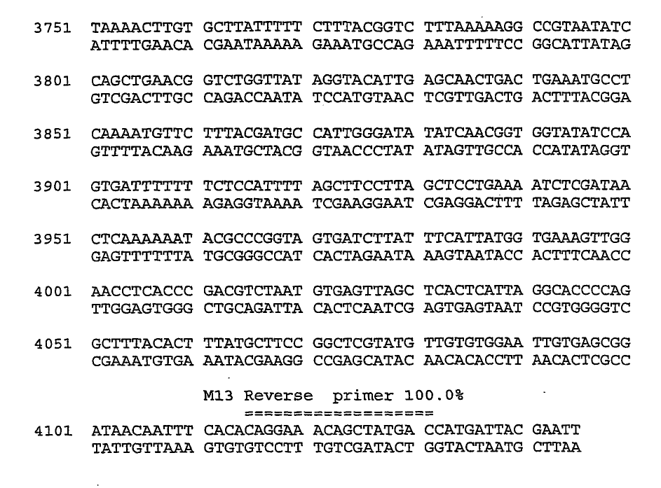

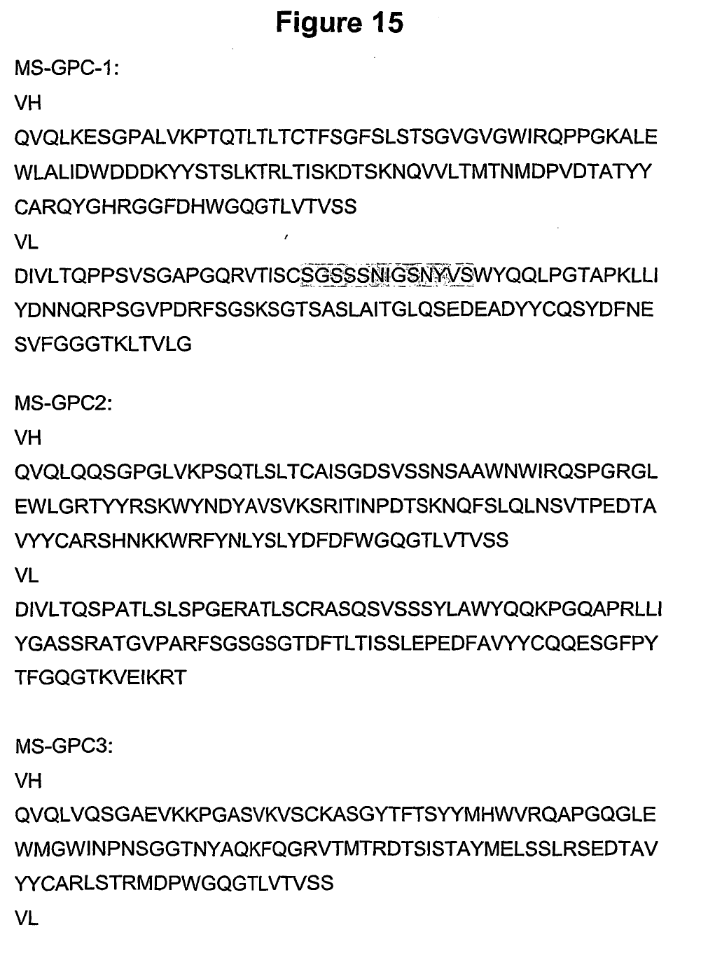

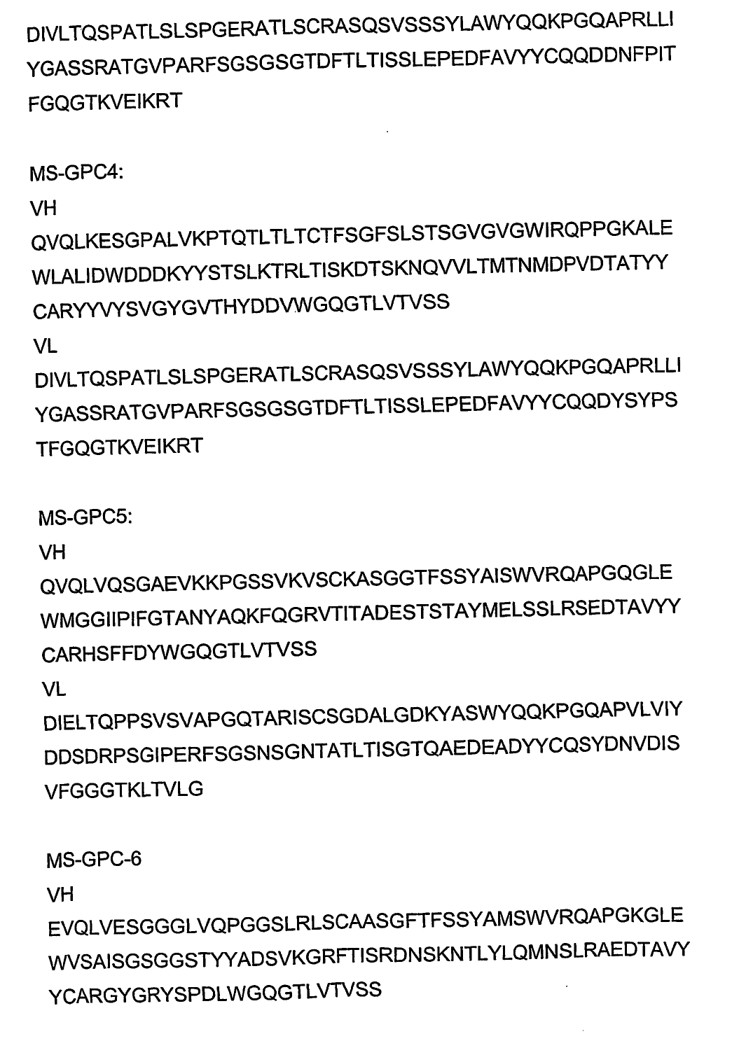

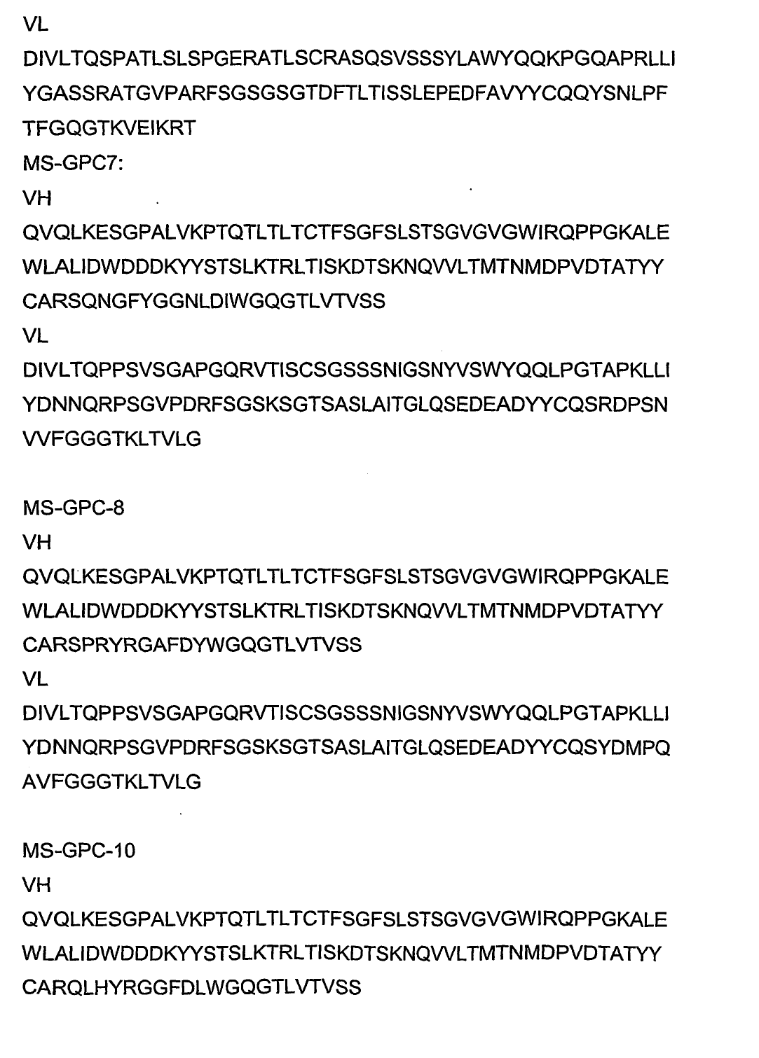

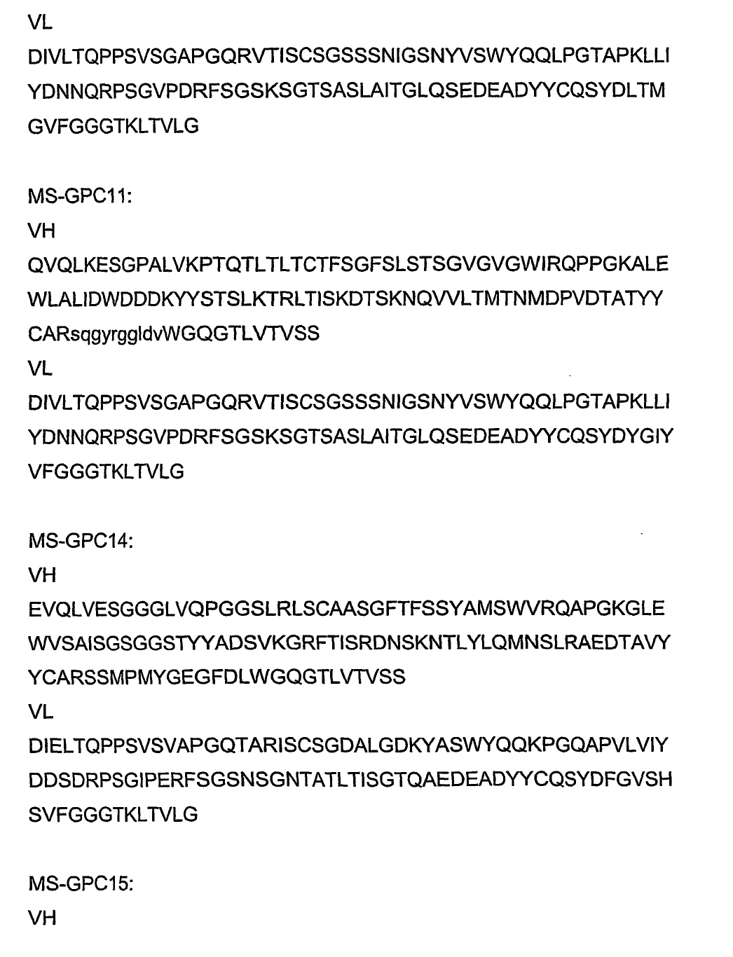

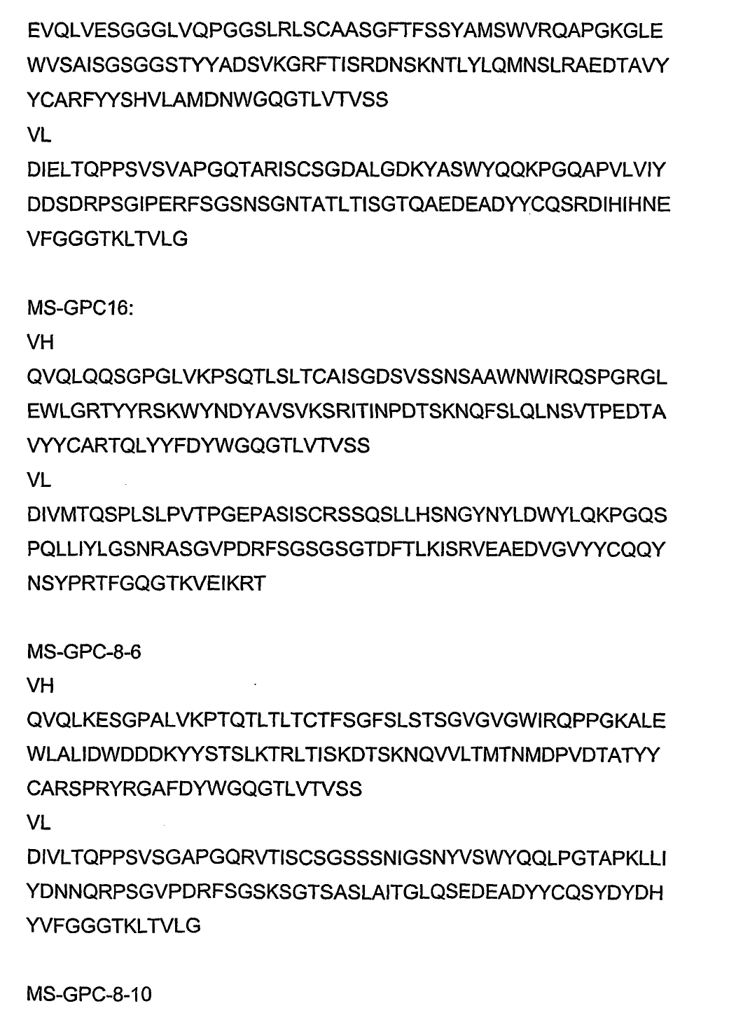

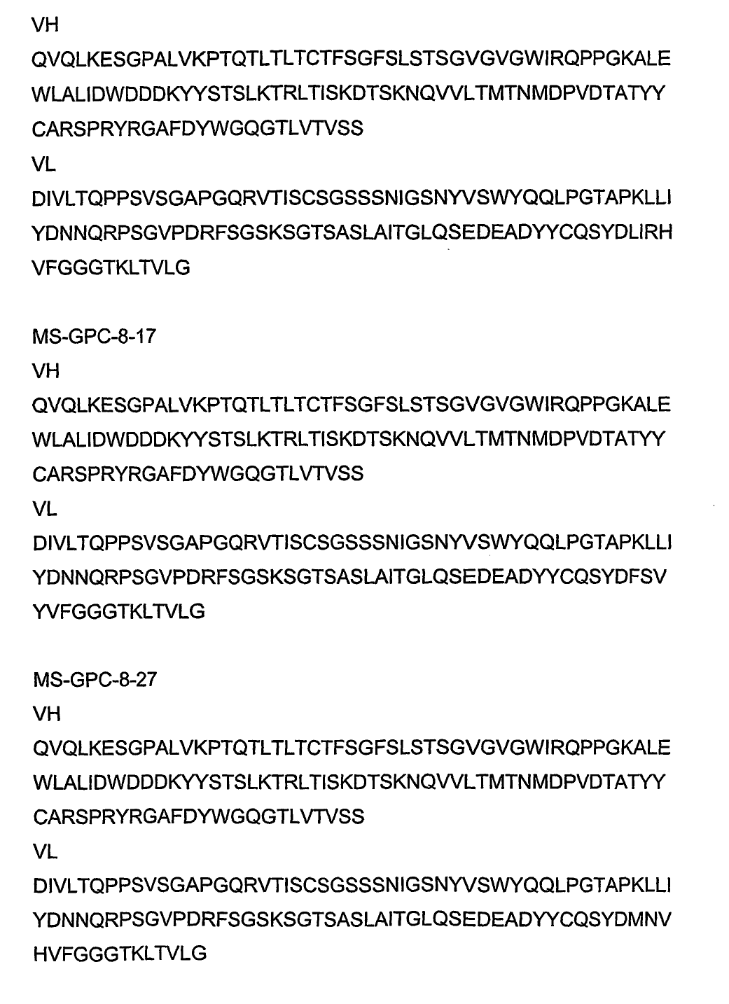

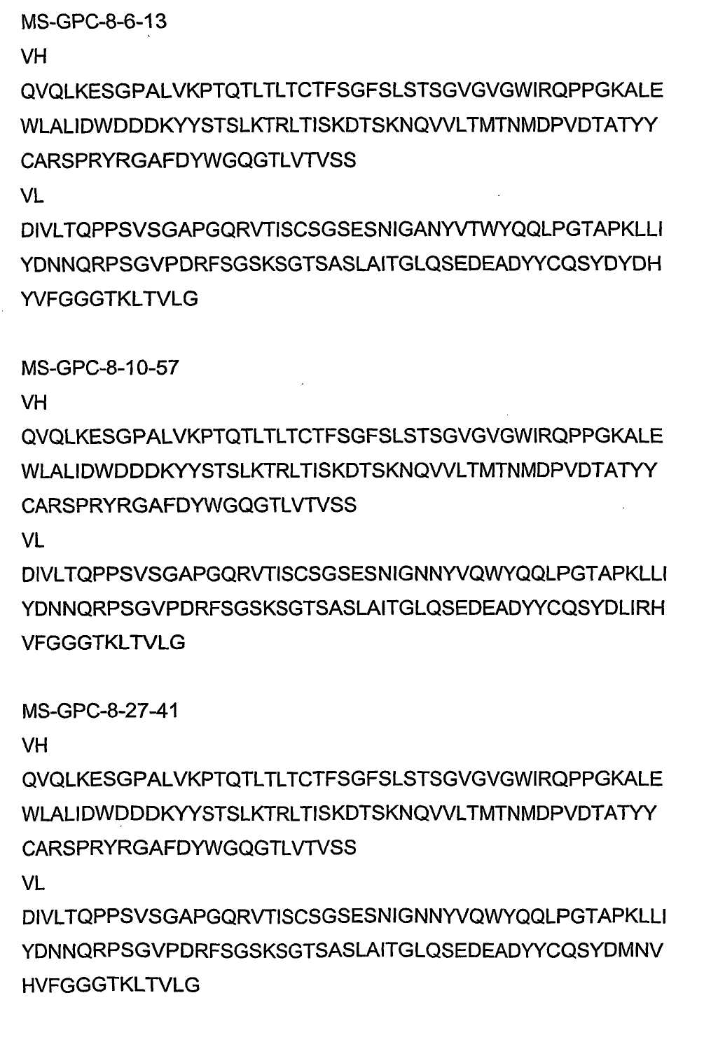

All anti-HLA-DR antibody fragments MS-GPC-8-27-7, MS-GPC-8-27-10, MS-GPC-8-6-13, MS-GPC-8-27-41, MS-GPC-8-6-47, MS-GPC-8-10-57, MS-GPC-8-6-27, MS-GPC-8 and MS-GPC-8-6 demonstrated high specificity for HLA-DR, as evidenced by the much higher fluorescence intensity resulting from incubation of these antibody fragments with HLA-DR derived antigens compared to controls (FIG. 1a). In a similar experiment, the Fab fragments MS-GPC-1, 2, 3, 4, 5, 6, 7, 8, 10, 11, 14, 15 & 16 were found to bind to both the DRA*0101/DRB1*0401 (prepared as above) as well as to a chimeric DR-IE consisting of the N-terminal domains of DRA*0101 and DRB1*0401 with the remaining molecule derived from a murine class II homologue IEd (Ito et al., 1996) (FIG. 1b).

To demonstrate the broad-DR reactivity of anti-HLA-DR antibody fragments and IgGs of the invention, the scFv forms of MS-GPC-1, 2, 3, 4, 5, 6, 7, 8, 10,:11, 14, 15 & 16, and IgG forms of MS-GPC-8, MS-GPC-8-10-57, MS-GPC-8-27-51 & MS-GPC-8-6-13 were tested for reactivity against a panel of Epstein-Barr virus transformed B cell lines obtained from ECACC (Salisbury UK), each homozygous for one of the most frequent DR alleles in human populations (list of cell lines and alleles shown in FIG. 2). The antibody fragments were also tested for reactivity against a series of L cells transfected to express human class II isotypes other than DRB1: L105.1, L257.6, L25.4, L256.12 & L21.3 that express the molecules DRB3*0101, DRB4*0101, DP0103/0402, DP 0202/0201, and DQ0201/0602 respectively (Klohe et al., 1988).

Reactivity of an antigen-binding fragment to the panel of cell-lines expressing various MHC-class II molecules was demonstrated using an immunofluorescence procedure as for example, described by Otten et al (1997). Staining was performed on 2×10⁵ cells using an anti-FLAG M2 antibody as the second reagent against the M2 tag carried by each anti-HLA-DR antibody fragment and a fluorescein labelled goat anti-mouse Ig (BD Pharmingen, Torrey Pine, Calif., USA) as a staining reagent. Cells were incubated at 4° C. for 60 min with a concentration of 200 nM of the anti-HLA-DR antibody fragment, followed by the second and third antibody at concentrations determined by the manufacturers. For the IgG form, the second antibody was omitted and the IgG detected using a FITC-labeled mouse anti-human IgG4 (Serotec, Oxford, UK). Cells were washed between incubation steps. Finally the cells were washed and subjected to analysis using a FACS Calibur (BD Immunocytometry Systems, San Jose, Calif., USA).

FIG. 2 shows that the scFv-fragments MS-GPC-1, 2, 5, 6, 7, 8, 10, 11, 14, 15 & 16, and IgG forms of MS-GPC-8, MS-GPC-8-10-57, MS-GPC-8-27-51 & MS-GPC-8-6-13 react with all DRB1 allotypes tested, while MS-GPC-3 & 4 react with over 3 DRB1 allotypes tested. This observation taken together with the observation that all anti-HLA-DR antibody fragments react with chimeric DR-IE, suggests that all selected anti-HLA-DR antibody fragments recognize the extracellular first domain of the monomorphic DRa chain or a monomorphic epitope on extracellular first domain of the DRβ chain.

We then attempted to localize the binding domains of MS-GPC-8-10-57 and MS-GPC-8-27-41 further by examining competitive binding with murine antibodies for which the binding domains on HLA-DR are known. The murine antibodies L243 and LB3.1 are known to bind to the α1 domain, 1-1C4 and 8D1 to the β1 domain and 10F12 to the β2 domain (Vidovic et al. 1995b). To this end, an assay was developed wherein a DR-expressing cell line (LG-2) was at first incubated with the IgG4 forms of MS-GPC-8-10-57 or MS-GPC-8-27-41, the Fab form of MS-GPC-8-10-57 or the Fab form of GPC 8, and an unrelated control antibody. Subsequently murine antibodies were added and the murine antibodies were detected. If the binding site of MS-GPC-8-10-57 or MS-GPC-8-27-41 overlaps with the binding of a murine antibody, then a reduced detection of the murine antibody is expected.

Binding of the IgG4 forms of GPC-8-27-41 and MS-GPC-8-10-57 and the Fab form of MS-GPC-8-10-57 substantially inhibited (mean fluorescence intensity reduced by >90%) the binding of 1-1C4 and 8D1, whereas L243, LB3.1 and 10F12 and a control were only marginally affected. The Fab form of MS-GPC-8 reduced binding of 1-1C4 by ˜50% (mean fluorescence dropped from 244 to 118), abolished 8D1 binding and only marginally affected binding of L243, LB3.1 and 10F12 or the control. An unrelated control antibody had no effect on either binding. Thus, MS-GPC-8-10-57 and MS-GPC-8-27-41 seem to recognise a β1 domain epitope that is highly conserved among allelic HLA-DR molecules.

The whole staining procedure was performed on ice. 1×10⁷ cells of the human B-lymphoblastoid cell line LG-2 was preblocked for 20 Min. in PBS containing 2% FCS and 35 μg/ml Guinea Pig IgG (“FACS-Buffer”). These cells were divided into 3 equal parts A, B, and C of approximately 3.3×10⁶ cells each, and it was added to A.) 35 μg MS-GPC-8-10-57 or MS-GPC-8-27-41 IgG4, to B.) 35 μg MS-GPC-8-10-57 Fab or MS-GPC-8 Fab, and to C.) 35 μg of an unrelated IgG4 antibody as negative control, respectively, and incubated for 90 min. Subsequently A, B, C were divided in 6 equal parts each containing 5.5×10⁵ cells, and 2 μg of the following murine antibodies were added each to one vial and incubated for 30 min: 1.) purified mIgG; 2.) L243; 3.) LB3.1; 4.) 1-1 C4; 5.)8D1; 6.) 10F12.Subsequently, 4 ml of PBS were added to each vial, the vials were centrifuged at 300 g for 8 min, and the cell pellet resuspended in 50 μl FACS buffer containing a 1 to 25 dilution of a goat-anti-murine Ig-FITC conjugate at 20 μg/ml final concentration (BD Pharmingen, Torrey Pines, Calif., USA). Cells were incubated light-protected for 30 min. Afterwards, cells were washed with 4 ml PBS, centrifuged as above, and resuspended in 500 μl PBS for analysis in the flow cytometer (FACS Calibur, BD Immunocytometry Systems, San Jose, Calif., USA).

The PepSpot technique (U.S. Pat. No. 6,040,423; Heiskanen et al., 1999) is used to further identify the binding epitope for MS-GPC 8-10-57. Briefly, an array of 73 overlapping 15 mer peptides is synthesised on a cellulose membrane by a solid phase peptide synthesis spotting method (WO 00/12575). These peptide sequences are derived from the sequence of the α1 and β1 domains of HLA-DR4Dw14, HLA-DRA1*0101 (residues 1-81) and HLA-DRB1*0401 (residues 2-92), respectively, and overlap by two amino acids. Second, such an array is soaked in 0.1% Tween-20/PBS (PBS-T), blocked with 5% BSA in PBS-T for 3 hours at room temperature and subsequently washed three times with PBS-T. Third, the prepared array is incubated for 90 minutes at room temperature with 50 ml of a 5 mg/l solution of the IgG form of GPC-8-10-57 in 1% BSA/PBS-T. Fourth, after binding, the membrane is washed three times with PBS-T and subsequently incubated for 1 hour at room temperature with a goat anti-human light chain antibody conjugated to horseradish peroxidase diluted 1/5000 in 1% BSA/PBS-T. Finally, the membrane is washed three times with PBS-T and any binding determined using chemiluminescence detection on X-ray film. As a control for unspecific binding of the goat anti-human light chain antibody, the peptide array is stripped by the following separate washings each at room temperature for 30 min: PBS-T (2 times), water, DMF, water, an aequeous solution containing 8M urea, 1 % SDS, 0.5% DTT, a solution of 50% ethanol, 10% acetic acid in water (3 times each) and, finally, methanol (2 times). The membrane is again blocked, washed, incubated with goat anti-human I light chain antibody conjugated to horseradish peroxidase and developed as described above.

7. Affinity of Anti-HLA-DR Antibody and Antibody Fragments

In order to demonstrate the superior binding properties of anti-HLA antibody fragments of the invention, we measured their binding affinities to the human MHC class II DR protein (DRA*0101/DRB1*0401) using standard equipment employing plasmon resonance principles. Surprisingly, we achieved affinities in the sub-nanomolar range for IgG forms of certain anti-HLA-DR antibody fragments of the invention. For example, the affinity of the IgG forms of MS-GPC-8-27-41, MS-GPC-8-6-13 & MS-GPC-8-10-57 was measured as 0.3, 0.5 and 0.6 nM respectively (Table 3a). Also, we observed high affinities in the range of 2-8 nM for Fab fragments affinity matured at the CDR1 and CDR3 light chain regions. (Table 3b). Fab fragments affinity matured at only the CDR3 light chain region showed affinities in the range of 40 to 100 nM (Table 3c), and even Fab fragments of non-optimised HuCAL antigen binding domains showed affinities in the sub μM range (Table 3d). We were surprised to observe that despite only a moderate increase in Kₒₙ (2-fold) following CDR3 optimisation, Kₒₙ remained approximately constant throughout the antibody optimisation process in the order of 1×10⁵ M⁻¹s⁻¹, whilst a significant decrease in K_{off} was a feature of the optimisation process—sub 100 s⁻¹, sub 10 s⁻¹, sub 1 s⁻¹ and sub 0.1 s⁻¹ for the unoptimised Fabs, CDR3 optimised Fabs, CDR3/CDR1 optimised Fabs and IgG forms of anti-HLA-DR antibody fragments of the invention.

The affinities for anti-HLA antibody fragments of the invention were measured as follows. All measurements were conducted in HBS buffer (20 mM HEPES, 150 mM NaCl, pH7.4) at a flow rate of 20 μl/min at 25° C. on a BIAcore3000 instrument (Biacore AB, Sweden). MHC class II DR protein (prepared as example 1) was diluted in 100 mM sodium acetate pH 4.5 to a concentration of 50 -100 mg/ml, and coupled to a CM5 chip (Biacore AB) using standard EDC-NHS coupling chemistry with subsequent ethanolamine treatment as manufacturers directions. The coating density of MHCII was adjusted to between 500 and 4000 RU. Affinities were measured by injection of 5 different concentrations of the different antibodies and using the standard software of the Biacore instrument. Regeneration of the coupled surface was achieved using 10 mM glycine pH2.3 and 7.5 mM NaOH.

8. Multivalent Killing Activity of Anti HLA-DR Antibodies and Antibody Fragments

To demonstrate the effect of valency on cell killing, a cell killing assay was performed using monovalent, bivalent and multivalent compositions of anti-HLA-DR antibody fragments of the invention against GRANTA-519 cells. Anti-HLA-DR antibody fragments from the HuCAL library showed much higher cytotoxic activity when cross-linked to form a bivalent composition (60-90% killing at antibody fragment concentration of 200 nM) by co-incubation with anti-FLAG M2 mAb (FIG. 3) compared to the monovalent form (5-30% killing at antibody fragment concentration of 200 nM). Incubation of cell lines alone or only in the presence of anti-FLAG M2 mAb without co-incubation of anti-HLA-DR antibody fragments did not lead to cytotoxicity as measured by cell viability. Treatment of cells as above but using 50 nM of the IgG4 forms (naturally bivalent) of the antibody fragments MS-GPC-8, MS-GPC-8-6-13, MS-GPC-8-10-57 and MS-GPC-8-27-41 without addition of anti-FLAG M2 mAb showed a killing efficiency after 4 hour incubation of 76%, 78%, 78% and 73% respectively.

Furthermore, we observed that higher order valences of the anti-HLA-DR antibody fragments further decrease cell viability significantly. On addition of Protein G to the incubation mix containing the IgG form of the anti-HLA-DR antibody fragments, the multivalent complexes thus formed further decrease cell viability compared to the bivalent composition formed from incubation of the anti-HLA-DR antibody fragments with only the bivalent IgG form.

The killing efficiency of anti-HLA-DR antibody fragments selected from the HuCAL library was tested on the HLA-DR positive tumor cell line GRANTA-519 (DSMZ, Germany). 2×10⁵ cells were incubated for 4 h at 37° C. under 6% CO₂ with 200 nM anti-HLA-DR antibody fragments in RPMI 1640 (PAA, Germany) supplemented with 2.5% heat inactivated FBS (Biowhittaker Europe, BE), 2 mM L-glutamine, 1% non-essential amino acids, 1 mM sodium pyruvate and 0.1 mg/ml kanamycin. Each anti-HLA-DR antibody fragment was tested for its ability to kill activated tumor cells as a monovalent anti-HLA-DR antibody fragment or as a bivalent composition by the addition of 100 nM of a bivalent cross-linking anti-FLAG M2 mAb. After 4 h incubation at 37° C. under 6% CO₂, cell viability was determined by trypan blue staining and subsequent counting of remaining viable cells (Current Protocols in Immunology, 1997).

The above experiment was repeated using KARPAS-422cells against a multivalent form of IgG forms of MS-GPC-8-10-57 and MS-GPC-8-27-41 prepared by a preincubation with a dilution series of the bacterial protein Protein G. Protein G has a high affinity and two binding sites for IgG antibodies, effectively cross-linking them to yield a total binding valency of 4. In a control using IgG alone without preincubation with Protein G, approximately 55% of cells were killed, while cell killing using IgG preincubated with Protein G gave a maximum of approximately 75% at a molar ratio of IgG antibody/Protein G of ˜6 (based on a molecular weight of Protein G of 28.5 kD). Higher or lower molar ratios of IgG antibody/Protein G approached the cell killing efficiency of the pure IgG antibodies.

9. Killing Efficiency of Anti-HLA-DR Antibody Fragments

Experiments to determine the killing efficiency of the anti-HLA-DR cross-linked antibody fragments against other tumor cell lines that express HLA-DR molecules were conducted analogous to example 8. Tumor cell lines that show greater than 50% cell killing with the cross linked Fab form of MS-GPC-8 after 4 h incubation include MHH-CALL4, MN 60, BJAB, BONNA-12 which represent the diseases B cell acute lymphoid leukemia, B cell acute lymphoid leukemia, Burkitt lymphoma and hairy cell leukemia respectively. Use of the cross-linked Fab form of the anti-HLA-DR antibody fragments MS-GPC-1, 6 and 10 also shows similar cytotoxic activity to the above tumor cell lines when formed as a bivalent agent using the cross-linking anti-FLAG M2 mAb.

The method described in example 8 was used to determine the maximum killing capacity for each of the cross-linked bivalent anti-HLA-DR antibody fragments against Priess cells. The maximum killing capacity observed for MS-GPC-1, MS-GPC-6, MS-GPC-8 & MS-GPC-10 was measured as 83%, 88%, 84% and 88% respectively. Antibody fragments generated according to example 4, when cross linked using anti-FLAG M2 mAb as above, also showed improved killing ability against GRANTA and Priess cells (Table 4).

10. Killing Efficiency of Anti-HLA-DR IgG Antibodies of Human Composition

Compared to corresponding murine antibodies (Vidovic et al, 1995b; Nagy & Vidovic, 1996; Vidovic & Toral; 1998), we were surprised to observe significantly improved killing efficiency of IgG forms of certain anti-HLA-DR antibody fragments of the invention (Table 5). Following the method described in examples 8 and 9 but at 50 nM, repeated measurements (3 to 5 replica experiments where cell number was counted in duplicate for each experiment) were made of the killing efficiency of the IgG forms of certain antibody fragments of the invention. When applied at a final concentration of only 50 nM, IgGs of the antibody fragments MS-GPC-8, MS-GPC-8-6-13, MS-GPC-8-10-57 & MS-GPC-8-27-41 killed more than 50% of cells from 16, 22, 19 and 20 respectively of a panel of 24 human tumor cell lines that express HLA-DR antigen at a level greater than 10 fluorescent units as determined by example 11. Cells were treated with the two murine anti-HLA-DR antibodies L243 (Vidovic et al, 1995b) and 8D1 (Vidovic & Toral; 1998) at a significantly higher final concentration of mAb (200 nM), which reduced cell viability to a level below 50% viable cells in only 13 and 12 of the 24 HLA-DR expressing cells lines, respectively. The cell line MHH-PREB-1 was singled out and not accounted as part of the panel of 24 cell lines despite its expression of HLA-DR antigen at a level greater than 10 fluorescent units due to the inability of any of the above antibodies to induce any significant reduction of cell viability. This is further explained in example 12.

Indeed, even at the significantly increased concentration, the two murine antibodies treated at 200 nM showed significantly efficient killing compared to the IgG forms of anti-HLA DR antibody fragments of the invention. Not only do IgG forms of the human anti-HLA-DR antibody fragments of the invention show an overall increase in cell killing compared to the murine antibodies, but they show less variance in killing efficiency across different cell lines. The coefficient of variance in killing for the human antibodies in this example is 32% (mean % killing=68±22% (SD)), compared to over 62% (mean % killing=49±31% (SD)) for the mouse antibodies. Statistically controlling for the effect on killing efficiency due to HLA expression by fifting logistic regression models to mean percentage killing against log(mean HLA DR expression) supports this observation (FIG. 4). Not only is the fitted curve for the murine antibodies consitently leower than that for the human, but a larger variance in residuals from the murine antibody data (SD=28%) is seen compared to the variance in residuals from the human antibody data (16%).

11. Killing Selectivity of Antigen-Binding Domains Against a Human Antigen for Activated Versus Non-Activated Cells

Human peripheral B cells were used to demonstrate that human anti-HLA-DR mAb-mediated cell killing is dependent on cell-activation. 50 ml of heparinised venous blood was taken from an HLA-DR typed healthy donor and fresh peripheral blood mononuclear cells (PBMC) were isolated by Ficoll-Hypaque Gradient Centrifugation (Histopaque-1077; Sigma) as described in Current Protocols in Immunology (John Wiley & Sons, Inc.; 1999). Purified B cells (−5% of peripheral blood leukocytes) were obtained from around 5×10⁷ PBMC using the B-cell isolation kit and MACS LS⁺/NS⁺ columns (Miltenyi Biotec, Germany) according to manufacturers guidelines. Successful depletion of non-B cells was verified by FACS analysis of an aliquot of isolated B cells (HLA-DR positive and CD19 positive). Double staining and analysis is done with commercially available antibodies (BD Immunocytometry Systems, San Jose, Calif., USA) using standard procedures as for example described in Current Protocols in Immunology (John Wiley & Sons, Inc.; 1999). An aliquot of the isolated B bells was tested for the ability of the cells to be activated by stimulation with Pokeweed mitogen (PWM) (Gibco BRL, Cat. No. 15360-019) diluted 1:25 in RPMI 1640 (PAA, Germany) supplemented with 10% FCS (Biowhittaker Europe, BE), 2 mM L-glutamine, 1% non-essential amino acids, 1 mM sodium pyruvate and 0.1 mg/ml kanamycin by incubation at 37° C. under 6% CO₂ for three days. Successful activation was verified by FACS analysis of HLA-DR expression on the cell surface (Current Protocols in Immunology, John Wiley & Sons, Inc.; 1999).

The selectivity for killing of activated cells versus non-activated cells was demonstrated by incubating 1×10⁶/ml B cells activated as above compared to non-activated cells, respectively with 50 nM of the IgG forms of MS-GPC-8-10-57, MS-GPC-8-27-41 or the murine IgG 10F12 (Vidovic et al., 1995b) in the medium described above but supplemented with 2.5% heat inactivated FCS instead of 10%, or with medium alone. After incubation at 37° C. under 6% CO₂ for 1 or 4 h, cell viability was determined by fluorescein diacetate staining (FDA) of viable and propidium iodide staining (PI) of dead cells and subsequent counting of the green (FDA) and red (PI) fluorescent cells using a fluorescence microscope (Leica, Germany) using standard procedures (Current Protocols in Immunology, 1997).

B cell activation was shown to be-necessary for cell killing. In non-activatedecells after 1 h of incubation with the anti-HLA-DR antibodies, the number of viable cells in the media corresponded to 81%, 117% 126% and 96% of the pre-incubation cell density for MS-GPC-8-10-57 (IgG), MS-GPC-8-27-41 (IgG), 10F12 and medium alone, respectively. In contrast, the number of viable activated B cells after 1 h incubation corresponded to 23%, 42% 83% and 66% of the pre-incubation cell density for MS-GPC-8-10-57 (IgG), MS-GPC-8-27-41 (IgG), 10F12 and medium alone, respectively. After 4 h of incubation, 78%, 83% 95% and 97% of the pre-incubation cell density for MS-GPC-8-10-57 (IgG), MS-GPC-8-27-41 (IgG), 1OF12 and medium alone were found viable in non-activated cells, whereas the cell density had dropped to 23%, 24% 53% and 67% of the pre-incubabon cell density for MS-GPC-8-10-57 (IgG), MS-GPC-8-27-41 (IgG), 10F12 and medium alone, respectively, in activated cells.

12. Killing Activity of Anti-HLA Antibody Fragments Against the Cell Line MHH PreB 1

As evidenced in Table 5, we observed that our cross-linked anti-HLA-DR antibody fragments or IgGs did not readily kill a particular tumor cell line expressing HLA-DR at significant levels. We hypothesized that although established as a stable cell line, cells in this culture were not sufficiently activated. Therefore, we conducted an experiment to stimulate activity of the MHH preB1 cell line, using increased cell-surface expression of HLA-DR molecule as a marker of activation as follows.

Non-adherently growing MHH preB1 cells were cultivated in RPMI medium containing the following additives (all from Gibco BRL and Bio Whittaker): 10% FCS, 2 mM L-glutamine, 1% non-essential amino acids, 1 mM sodium pyruvate and 1× Kanamycin. Aliquots were activated to increase expression of HLA-DR molecule by incubation for one day with Lipopolysaccharide (LPS, 10 μg/ml), Interferon-gamma (IFN-γ, Roche, 40 ng/ml) and phyto-hemagglutinin (PHA, 5 μg/ml). The cell surface expression of HLA-DR molecules was monitored by flow cytometry with the FITC-conjugated mAb L243 (BD Immunocytometry Systems, San Jose, Calif., USA). Incubation of MHH preB1 for one day in the presence of LPS, IFN-γ and PHA resulted in a 2-fold increase in HLA-DR surface density (mean fluorescence shift from 190 to 390). Cell killing was performed for 4 h in the above medium but containing a reduced FCS concentration (2.5%). A concentration series of the IgG forms of MS-GPC-8-27-41 & MS-GPC-8-10-57 was employed, consisting of final antibody concentrations of 3300, 550, 92, 15, 2.5, 0.42 and 0.07 nM, on each of an aliquot of non-activated and activated cells. Viable cells were identified microscopically by exclusion of Trypan blue. Whereas un-activated cell viability remains unaffected by the antibody-up to the highest antibody concentration used, cell viability is dramatically reduced with increasing antibody concentration in activated MHH PreB1 cells (FIG. 5).

13. Killing Efficiency of Anti-HLA-DR IgG Antibodies of Human Composition Against Ex-Vivo Chronic Lymphoid Leukemia Cells

Using B cells isolated and purified from 10 patients suffering from chronic lymphoid leukemia (CLL), we demonstrated that IgG forms of anti-HLA-DR antibody fragments of the invention showed efficacy in killing of clinically relevant cells using an ex-vivo assay. B-cells were isolated and purified from 10 unrelated patients suffering from CLL (samples kindly provided by Prof Hallek, Ludwig Maximillian University, Munich) according to standard procedures (Scandinavian J. of Immunology 1968. I'll need to get that on Monday). 2×10⁵ cells were treated with 100 nM of IgG forms of the anti-HLA-DR antibody fragments MS-GPC-8, MS-GPC-8-10-57 or MS-GPC-8-27-41 and incubated for 4 or 24 hours analogous to examples 8 and 9. A replica set of cell cultures was established and activated by incubation with HeLa-cells expressing CD40 ligand on their surface for three days before treatment with antibody (Buhmann et al., 1999). As controls, the murine IgG 10F12 (Vidovic et al., 1995b) or no antibody was used. Cell viability for each experiment was determined as described in example 12.

Surprisingly, IgG forms of the anti-HLA-DR antibody fragments of the invention showed highly efficient and uniform killing—even across this diverse set of patient material. After only 4 hours of treatment, all three human IgGs gave a significant reduction in cell viability compared to the controls, and after 24 hours only 33% of cells remained viability (FIG. 6). We found that on stimulating the ex-vivo cells further according to Buhmann et al (1999), the rate of killing was increased such that after only 4 hours culture with the human antibodies, only 24% of cells remained viable on average for all patient samples and antibody fragments of the invention.

14. Determination of EC50 for Anti-HLA-DR Antibody Fragments

We demonstrated superior Effective Concentration at 50% effect (EC50) values in a cell-killing assay for certain forms of anti-HLA-DR antibody fragments selected from the HuCAL library compared to cytotoxic murine anti-HLA-DR antibodies (Table 6).

The EC50 for anti-HLA-DR antibody fragments selected from the HuCAL library were estimated using the HLA-DR positive cell line PRIESS or LG2 (ECACC, Salisbury UK). 2×10⁵ cells were incubated for 4 h at 37° C. under 6% CO₂ in RPMI 1640 (PAA, Germany) supplemented with 2.5% heat inactivated FBS (Biowhittaker Europe, BE), 2mM L-glutamine, 1% nonessential amino acids, 1 mM sodium pyruvate and 0.1 mg/ml kanamycin, together with dilution series of bivalent anti-HLA-DR antibody fragments. For the dilution series of Fab antibody fragments, an appropriate concentration of Fab fragment and anti-FLAG M2 antibody were premixed to generate bivalent compositions of the anti-HLA-DR antibody fragments. The concentrations stated refer to the concentration of bivalent composition such that the IgG and Fab EC50 values can be compared.

After 4 h incubation with bivalent antibody fragments at 37° C. under 6% CO₂, cell viability was determined by fluorescein diacetate staining and subsequent counting of remaining viable cells (Current Protocols in Immunology, 1997). Using standard statistical software (R; http://cran.r-project.org), non-linear logistic regression curves were fitted to replica data points and the EC50 estimated for each antibody fragment.

When cross-linked using the anti-FLAG M2 antibody, the Fab fragments MS-GPC-1, MS-GPC-8 & MS-GPC-10 selected from the HuCAL library (Example 4) showed an EC50 of less than 120 nM as expressed in terms of the concentration of the monovalent fragments, which corresponds to a 60 nM EC50 for the bivalent cross-linked (Fab)dimer-anti-Flag M2 conjugate. (FIG. 7a). When cross-linked using the anti-FLAG M2 antibody, anti-HLA-DR antibody fragments optimised for affinity within the CDR3 region (Example 4) showed a further improved EC50 of less than 50 nM, or 25 nM in terms of the bivalent cross-linked fragment (FIG. 7b), and those additionally optimised for affinity within the CDR1 region showed an EC50 of less than 30 nM (15 nM for bivalent fragment). In comparison, the EC50 of the cytotoxic murine anti-HLA-DR antibodies 8D1 (Vidovic & Toral; 1998) and L243 (Vidovic et al; 1995b) showed an EC50 of over 30 and 40 nM, respectively, within the same assay (FIG. 7c).

Surprisingly, the IgG form of certain antibody fragments of the invention showed approximately 1.5 orders of magnitude improvement in EC50 compared to the murine antibodies (FIG. 7d). For example, the IgG forms of MS-GPC-8-10-57 & MS-GPC-8-27-41 showed an EC50 of 1.2 and 1.2 nM respectively. Furthermore, despite being un-optimised for affinity, the IgG form of MS-GPC-8 showed an EC50 of less than 10 nM.

As has been shown in examples 11 and 12, the efficiency of killing of un-activated cells (normal peripheral B and MHH PreB cells respectively) is very low. After treatment with 50 nM of the IgG forms of MS-GPC-8-10-57 & MS-GPC-8-27-41, 78% and 83% of normal peripheral B cells, respectively, remain viable after 4 hours. Furthermore, at only 50 nM concentration or either IgG, virtually 100% viability is seen for MHH PreB1 cells. Indeed, a decrease in the level of viability to below 50% cannot be achieved with these un-activated cells using reasonable concentration ranges (0.1 to 300 nM) of IgG or bivalent cross-linked Fab forms of the anti-HLA DR antibody fragments of the invention. Therefore, the EC50 for these un-activated cell types can be estimated to be at least 5 times higher than that shown for the non-optimised Fab forms (EC50 ˜60 nM with respect to cross-linked bivalent fragment), and at least 10 times and 100 times higher than EC50s shown for the VHCDR3 optimised Fabs (˜25 nM with respect to cross-linked bivalent fragment) and IgG forms of MS-GPC-8-10-57 (˜1.2 nM) & MS-GPC-8-27-41 (˜1.2 nM) respectively.

15. Mechanism of Cell-Killing

The examples described above show that cell death occurs—needing only certain multivalent anti-HLA-DR antibody fragments to cause killing of activated cells. No further cytotoxic entities or immunological mechanisms were needed to cause cell death, therefore demonstrating that cell death is mediated through an innate pre-programmed mechanism of the activated cell. The mechanism of apoptosis is a widely understood process of pre-programmed cell death. We were surprised by certain characteristics of the cell killing we observed that suggested the mechanism of killing for activated cells when exposed to our human anti-HLA-DR antibody fragments was not what is commonly understood in the art as “apoptosis”. For example, the observed rate of cell killing appeared to be significantly greater than the rate reported for apoptosis (reference; I still need to get that from Zoltan on Monday). Two experiments were conducted to demonstrate that the mechanism of cell killing proceeded by a non-apoptotic mechanism.

First, we used Annexin-V-FITC and propidium iodide (PI) staining techniques to distinguish between apoptotic and non-apoptotic cell death—cells undergoing apoptosis, “apoptotic cells”, (Annexin-V positive/PI negative) can be distinguished from necrotic (“Dead”) (Annexin-V positive/PI positive) and fully functional cells (Annexin-V negative/PI negative). Using the procedures recommended by the manufacturers of the AnnexinV and Pi assays, 1×10⁶/ml Priess cells were incubated at 37° C. under 6% CO₂ with or without 200 nM anti-HLA-DR antibody fragment MS-GPC-8 together with 100 nM of the cross-linking anti-FLAG M2 mAb in RPMI 1640 (PAA, DE) supplemented with 2.5% heat inactivated FCS (Biowhittaker Europe, BE), 2 mM L-glutamine; 1% non-essential amino acids, 1 mM sodium pyruvate and 0.1 mg/ml kanamycin. To provide an apoptotic cell culture as control, 1×10⁶/ml Priess cells were induced to enter apoptosis by incubation in the above medium at 37° C. under 6% CO₂ with 50 μg/ml of the apoptosis-inducing anti-CD95 mAb DX2 (BD Pharmingen, Torrey Pine, Calif., USA) cross-linked with 10 μg/ml Protein-G. At various incubation times (1, 15 and 60 min, 3 and 5 h) 200 μl samples were taken, washed twice and stained with Annexin-V-FITC (BD Pharmingen, Torrey Pine, Calif., USA) and PI using Annexin-V binding buffer following the manufacturer's protocol. The amount of staining with Annexin-V-FITC and PI for each group of cells is analysed with a FACS Calibur (BD Immunocytometry Systems, San Jose, Calif., USA).

Cell death induced through the cross-linked anti-HLA-DR antibody fragments shows a significantly different pattern of cell death than that of the anti-CD95 apoptosis inducing antibody or the cell culture incubated with anti-FLAG M2 mAb alone. The percentage of dead cells (as measured by Annexin-V positive/PI positive staining) for the anti-HLA-DR antibody fragment/anti-FLAG M2 mAb treated cells increases far more rapidly than that of the anti-CD95 or the control cells (FIG. 8a). In contrast, the percentage of apoptotic cells (as measured by Annexin-V positive/PI negative staining) increases more rapidly for the anti-CD95 treated cells compared to the cross-linked anti-HLA-DR antibody fragments or the control cells (FIG. 8b).

Second, we inhibited caspase activity using zDEVD-fmk, an irreversible Caspase-3 inhibitor, and zVAD-fmk, a broad spectrum Caspase inhibitor (both obtained from BioRad, Munich, DE). The mechanism of apoptosis is characterized by activity of caspases, and we hypothesized that if caspases were not necessary for anti HLA-DR mediated cell death, we would observe no change in the viability of cells undergoing cell death in the presence of these caspase inhibitors compared to those without. 2×10⁵ Priess cells were preincubated for 3 h at 37° C. under 6% CO₂ with serial dilutions of the two caspase inhibitors ranging from 180 μM to 10 mM in RPMI 1640 (PAA, DE) supplemented with 2.5% heat inactivated FCS (Biowhittaker Europe, BE), 2 mM L-glutamine, 1% non-essential amino acids, 1 mM sodium pyruvate and 0.1 mg/ml kanamycin. HLA-DR mediated cell death was induced by adding 200 nM of the human anti-HLA-DR antibody fragment MS-GPC-8 and 100 nM of the cross-linking anti-M2 mAb. An anti-CD95 induced apoptotic cell culture served as a control for the activity of inhibitors (Drenou et al., 1999). After further incubation at 37° C. and 6% CO₂, cell viability after 4 and 24 h was determined by trypan blue staining and subsequent counting of non-stained cells. As we expected, cell viability of the anti-HLA-DR treated cell culture was not significantly modified by the presence of the Caspase inhibitors, while cell death induced through anti-CD95 treatment was significantly decreased for the cell culture pre-incubated with the Caspase inhibitors. This observation supports our hypothesis that HLA-DR mediated cell death proceeds through a non-apoptotic mechanism that is independent of caspase proteases that can be inhibited by zDEVD-fm or zVAD-fmk.

16. In Vivo Therapy for Cancer Using an HLA-DR Specific Antibody

We demonstrate that antigen-binding domains of human composition can successfully be used as a therapeutic for the treatment of cancer. Immunocompromised mice—such as scid, nude or Rag-1 knockout—are inoculated with a DR+ human lymphoma or leukemia cell line of interest. The tumor cell dose, usually 1×10⁶ to 1×10⁷/mouse, is established for each tumor tested and administered subcutaneously (s.c.) or intravenously (i.v.). The mice are treated i.v. or s.c with the IgG form of the anti-HLA-DR antibody fragments MS-GPC-8, MS-GPC-8-10-57, MS-GPC-8-27-41 or others of the invention prepared as described above, using doses of 1 to 25 mg/kg over 5 days. Survival of anti-HLA-DR treated and control untreated mice is monitored for up to 8 weeks after cessation of treatment. Tumor progression in the mice inoculated s.c. is additionally quantified by measuring tumor surface area. Significant prolongation of survival of up to 80% of anti-HLA-DR treated mice is observed during the experiment, and up to 50% mice survive at the end of the experiment. In s.c. inoculated and untreated mice, the tumor reaches a surface area of 2-3 cm², while in anti-HLA-DR treated animals the tumor surface area is significantly less.

17. Immunosuppression Using Anti-HLA-DR Antibody Fragments Measured by Reduction in IL-2 Secretion

Anti-HLA DR antibody fragments of the invention displayed substantial immunomodulatory properties within an assay measuring IL-2 secretion from immortalized T-cells. IgG forms of the antibody fragments MS-GPC-8-6-13, MS-GPC-8-10-57 & MS-GPC-8-27-41 showed very strong immunosuppressive properties in this assay with sub-nanomolar IC50 values and virtually. 100% maximal inhibition (FIG. 9a). Surprisingly, even monvalent compositions of the antibody fragments of the invention were able to strongly inhibit IL-2 secretion in the same assay. For example, Fab forms of the VHCDR3-selected and VLCDR3/VLCDR1 optimised antibody fragments showed low single-digit nano-M IC50s and also almost 100% maximal inhibition (FIG. 9b). Other monvalent anti-HLA DR antibody fragments of the invention showed significant immunosuppressive properties in the assay compared to control IgG and Fab fragments (Table 7).

The immunomodulatory properties of anti-HLA DR antibody fragments was investigated by measuring IL-2 secretion from the hybridoma cell line T-Hyb 1 stimulated using DR-transgenic antigen presenting cells (APC) under conditions of half-maximal antigen stimulation. IL-2 secretion was detected and measured using a standard ELISA method provided by the OptiEIA mouse IL-2 kit of Pharmingen (Torrey Pine, Calif., USA). APCs were isolated from the spleen of unimmunized chimeric 0401-IE transgenic mice (Ito et al. 1996) according to standard procedures.

1.5×10⁵ APCs were added to 0.2 ml wells of 96well in RPMI medium containing the following additives (all from Gibco BRL and PAA): 10 % FCS, 2 mM L-glutamine, 1% non-essential amino acids, 1 mM sodium pyruvate and 0.1 g/l kanamycin. Hen egg ovalbumin was added to a final concentration of 200 μg/ml in a final volume of 100 μl of the above medium, the cells incubated with this antigen for 30 min at 37° C. under 6% CO₂. Anti-HLA DR antibody fragments were added to each well at various concentrations (typically in a range from 0.1 to 200 nM), the plate incubated for 1 h at 37° C./6% CO₂ and 2×10⁵ T-Hyb 1 cells added to give a final volume of 200 μl in the above medium. After incubation for 24 h, 100 μl of supernatant was transferred to an ELISA plate (Nunc-Immuno Plate MaxiSorp surface, Nunc, Roskilde, DK) previously coated with IL-2 Capture Antibody (BD Pharmingen, Torrey Pine, Calif., USA), the amount of IL-2 was quantified according to the manufacturer's directions using the OptiEIA Mouse IL-2 kit and the plate read using a Victor V reader (Wallac, Finland). Secreted IL-2 in pg/ml was calibrated using the IL-2 standards provided in the kit.

The T-cell hybridoma line T-Hyb1 was established by fusion of a T-cell receptor negative variant of the thymoma line BW 5147 (ATCC) and lymph node cells from chimeric 0401-IE transgenic mice previously immunized with hen egg ovalbumin (Ito et al. 1996). The clone T-Hyb1 was selected for the assay since it responded to antigen specific stimulation with high IL-2 secretion.

18. Immunosuppression Using an HLA-DR Specific Antibody Measured by T Cell Proliferation

Immunomodulatory properties of anti-HLA DR antibody fragments were confirmed using a second assay that measures T cell proliferation. The IC50 value for inhibition of T cell proliferation of the IgG form of MS-GPC-8-10-57 and MS-GPC-8-27-41 were 11 and 20 nM respectively (FIG. 10). The anti-H LA DR antibody fragments were tested as follows to inhibit the proliferative T cell response of antigen-primed lymph node cells from mice carrying a chimeric mouse-human class II transgene with an RA-associated peptide binding site, and lack murine class II molecules (Muller et al., 1990; Woods et al., 1994; Current Protocols in Immunology, Vol. 2, 7.21; Ito et al., 1996). Here, the immunization takes place in vivo, but the inhibition and readout are ex vivo. Transgenic mice expressing MHC class II molecules with binding sites of the RA associated molecule, DRB1* 0401 were previously generated (Ito et al 1996).

These mice lack murine MHC class II, and thus, all Th responses are channelled through a single human RA-associated MHC class II molecule (Ito et al. 1996). These transgenic mice represent a model for testing human class II antagonists.

The inhibitory effect of the anti-HLA-DR antibody fragments and their IgG forms were tested on T-cell proliferation measured using chimeric T-cells and antigen presenting cells isolated from the lymph nodes of chimeric 0401-IE transgenic mice (Taconic, USA) previously immunized with hen egg ovalbumin (Ito et al. 1996) according to standard procedures. 1.5×10⁵ cells are incubated in 0.2 ml wells of 96-well tissue culture plates in the presence of ovalbumin (30 μg per well—half-maximal stimulatory concentration) and a dilution series of the anti-HLA DR antibody fragment or IgG form under test (0.1 nM-200 nM) in serum free HL-1 medium containing 2 mM L-glutamine and 0.1 g/l Kanamycin for three days. Antigen specific proliferation is measured by ³H-methyl-thymidin(1 μCi/well) incorporation during the last 16h of culture (Falcioni et al., 1999). Cells are harvested, and ³H incorporation measured using a scintillation counter (TopCount, Wallac Finland). Inhibition of T-cell proliferation on treatment with the anti-HLA DR antibody fragment and its IgG form may be observed by comparison to control wells containing antigen.

19. Selection of Useful Polypeptide for the Treatment of Cancers

In order to select the most appropriate protein/peptide to enter further experiments and to assess its suitability for use in a therapeutic composition for the treatment of cancers, additional data are collected. Such data for each IgG form of the anti-HLA antigen antibody fragments can include the binding affinity, in vitro killing efficiency as measured by EC50 and cytotoxicity across a panel of tumor cell lines, the maximal percentage cell killing as estimated in vitro, and tumor reduction data and mouse survival data from in vivo animal models.

The IgG form of the anti-HLA antigen antibody fragments that shows the highest affinity, the lowest EC50 for killing, the highest maximal percentage cell killing and broadest across various tumor cell lines, the best tumor reduction data and/or the best mouse-survival data may be chosen to enter further experiments. Such experiments may include, for example, therapeutic profiling and toxicology in animals and phase I clinical trials in humans.

20. Selection of Useful Polypeptide for the Treatment of Diseases of the Immune System

In order to select the most appropriate protein/peptide to enter further experiments and to assess its suitability for use in a therapeutic composition for the treatment of diseases of the immune system, additional data are collected. Such data for each monovalent antibody fragment or IgG form of the anti-HLA antigen antibody fragments can include the affinity, reactivity, specificity, IC50-values, for inhibition of IL-2 secretion and of T-cell proliferation, or in vitro killing efficiency as measured by EC50 and the maximal percentage cell killing as estimated in vitro, and DR-transgenic models of transplant rejection and graft vs. host disease.

The antibody fragment or IgG form of the anti-HLA antigen antibody fragments that shows the lowest EC50, highest affinity, highest killing, best specificity and/or greatest inhibition of T-cell proliferation or IL-2 secretion, and high efficacy in inhibiting transplant rejection and/or graft vs. host disease in appropriate models, might be chosen to enter further experiments. Such experiments may include, for example, therapeutic profiling and toxicology in animals and phase I clinical trials in humans.

TABLE 1
VH and VL families, VL CDR1 and VH/VL CDR 3 sequences of HLA-DR-specific
polypeptides

| | | CDR3 | | | | CDR3 | | |
|---|---|---|---|---|---|---|---|---|
| Clone | VH | Length | VH-CDR3-Seq. | VL | VL-CDR1-Seq. | Length | VL-CDR3-Seq. | Families |
| MS-GPC-1 | H2 | 10 | QYGHRGGFDH | Λ1 | SGSSSNIGSNYVS | 8 | QSYDFNES | H2 λ1 |
| MS-GPC2 | H6 | 19 | SHNKKWRFYNLY | κ3 | RASQSVSSSYLA | 8 | QQESGFPY | H6κ3 |
| | | | SLYDFDF | | | | | |
| MS-GPC3 | H1B | 7 | LSTRMDP | κ3 | RASQSVSSSYLA | 8 | QQDDNFPI | H1Bκ3 |
| MS-GPC4 | H2 | 16 | YYVYSVGYGVTHY | κ3 | RASQSVSSSYLA | 8 | QQDYSYPS | H2κ3 |
| | | | DDV | | | | | |
| MS-GPC5 | H1A | 6 | HSFFDY | λ3 | | 9 | QSYDNVDIS | H1Aλ3 |
| MS-GPC-6 | H3 | 9 | GYGRYSPDL | K3 | RASQSVSSSYLA | 8 | QQYSNLPF | H3 K3 |
| MS-GPC7 | H2 | 13 | SQNGFYGGNLDI | λ1 | SGSSSNIGSNYVS | 8 | QSRDPSNV | H2λ1 |
| MS-GPC-8 | H2 | 10 | SPRYRGAFDY | λ1 | SGSSSNIGSNYVS | 8 | QSYDMPQA | H2 λ1 |
| MS-GPC-10 | H2 | 10 | QLHYRGGFDL | λ1 | SGSSSNIGSNYVS | 8 | QSYDLTMG | H2 λ1 |
| MS-GPC11 | H2 | 10 | SQGYRGGLDV | λ1 | SGSSSNIGSNYVS | 8 | QSYDYGIY | H2λ1 |
| MS-GPC14 | H3 | 12 | SSMPMYGEGFDL | λ3 | | 10 | QSYDFGVSHS | H3λ3 |
| MS-GPC15 | H3 | 10 | FYYSHVLAMDN | λ3 | | 10 | QSRDIHIHNE | H3λ3 |
| MS-GPC16 | H6 | 8 | TQLYYFDY | κ2 | | 8 | QQYNSYPR | H6κ2 |
| MS-GPC-8-1 | H2 | 10 | SPRYRGAFDY | λ1 | SGSSSNIGSNYVS | 8 | QSYDFSHY | H2 λ1 |
| MS-GPC-8-6 | H2 | 10 | SPRYRGAFDY | λ1 | SGSSSNIGSNYVS | 8 | QSYDYDHY | H2 λ1 |
| MS-GPC-8-9 | H2 | 10 | SPRYRGAFDY | λ1 | SGSSSNIGSNYVS | 8 | QSYDIQLH | H2 λ1 |
| MS-GPC-8-10 | H2 | 10 | SPRYRGAFDY | λ1 | SGSSSNIGSNYVS | 8 | QSYDLIRH | H2 λ1 |
| MS-GPC-8-17 | H2 | 10 | SPRYRGAFDY | λ1 | SGSSSNIGSNYVS | 8 | QSYDFSVY | H2 λ1 |
| MS-GPC-8-18 | H2 | 10 | SPRYRGAFDY | λ1 | SGSSSNIGSNYVS | 8 | QSYDFSIY | H2 λ1 |
| MS-GPC-8-27 | H2 | 10 | SPRYRGAFDY | λ1 | SGSSSNIGSNYVS | 8 | QSYDMNVH | H2 λ1 |
| MS-GPC-8-6-2 | H2 | 10 | SPRYRGAFDY | λ1 | SGSESNIGSNYVH | 8 | QSYDYDHY | H2 λ1 |
| MS-GPC-8-6-19 | H2 | 10 | SPRYRGAFDY | λ1 | SGSESNIGSNYVA | 8 | QSYDYDHY | H2 λ1 |
| MS-GPC-8-6-27 | H2 | 10 | SPRYRGAFDY | λ1 | SGSDSNIGANYVT | 8 | QSYDYDHY | H2 λ1 |
| MS-GPC-8-6-45 | H2 | 10 | SPRYRGAFDY | λ1 | SGSEPNIGSNYVF | 8 | QSYDYDHY | H2 λ1 |
| MS-GPC-8-6-13 | H2 | 10 | SPRYRGAFDY | λ1 | SGSESNIGANYVT | 8 | QSYDYDHY | H2 λ1 |
| MS-GPC-8-6-47 | H2 | 10 | SPRYRGAFDY | λ1 | SGSESNIGSNYVS | 8 | QSYDYDHY | H2 λ1 |
| MS-GPC-8-10-57 | H2 | 10 | SPRYRGAFOY | λ1 | SGSESNIGNNYVQ | 8 | QSYDLIRH | H2 λ1 |
| MS-GPC-8-27-7 | H2 | 10 | SPRYRGAFDY | λ1 | SGSESNIGNNYVG | 8 | QSYDMNVH | H2 λ1 |
| MS-GPC-8-27-10 | H2 | 10 | SPRYRGAFDY | λ1 | SGSESNIGANYVN | 8 | QSYDMNVH | H2 λ1 |
| MS-GPC-8-27-41 | H2 | 10 | SPRYRGAFDY | λ1 | SGSESNIGNNYVQ | 8 | QSYDMNVH | H2 λ1 |

TABLE 2

| Steps in Antibody | | kₒₙ [s⁻¹M⁻¹] × 10⁵ | k_{off} [S⁻¹] × 10⁻³ | K_{D} [nM] | | |
|---|---|---|---|---|---|---|
| optimisation | Fab | +/− SD | +/− SD | +/− SD | L-CDR3 | L-CRD1 |
| Parental Fab | MS-GPC-8 | 0.99 ± 0.40 | 29.0 ± 8.40 | 346.1 ± 140.5^{a)} | QSYDMPQA | SGSSSNIGSNYVS |
| L-CDR3-optim. | -8-1 | 1.93 | 20.9 | 108^{e)} | | |
| L-CDR3-optlm. | -8-6 | 0.96 ± 0.14 | 5.48 ± 0.73 | 58.6 ± 11.7^{b)} | | |
| L-CDR3-optim. | -8-9 | 1.85 | 16.6 | 90.1^{e)} | | |
| L-CDR3-optim. | -8-10 | nd | 7.0^{a)} | nd | | |
| L-CDR3-optlm. | -8-17 | 1.0 | 5.48 | 54.7^{e)} | | |
| L-CDR3-optim. | -8-18 | 1.06 | 8.3 | 78.3^{e)} | | |
| L-CDR3-optim. | -8-27 | nd | 6.6^{e)} | nd | | |
| L-CDR3-optim. | -8-6 | 0.96 ± 0.14 | 5.48 ± 0.73 | 58.6 ± 11.7^{b)} | QSYDYDHY | SGSSSNIGSNYVS |
| L-CDR3 + 1-opt. | -8-6-2 | 1.23 ± 0.11 | 0.94 ± 0.07 | 7.61 ± 0.25^{c)} | QSYDYDHY | SGSESNIGSNYVH |
| L-CDR3 + 1-opt. | -8-6-19 | 1.10 ± 0.08 | 0.96 ± 0.15 | 8.74 ± 1.33^{c)} | QSYDYDHY | SGSESNIGSNYVA |
| L-CDR3 + 1-opt. | -8-6-27 | 1.80 ± 0.24 | 1.10 ± 0.15 | 6.30 ± 0.63^{d)} | OSYDYDHY | SGSDSN1GANYVT |
| L-CDR3 + 1-opt. | -8-6-45 | 1.20 ± 0.07 | 1.03 ± 0.04 | 8.63 ± 0.61^{c)} | QSYDYDHY | SGSEPNIGSNYVF |
| L-CDR3 + 1-opt. | -8-6-13 | 1.90 ± 0.26 | 0.55 ± 0.05 | 2.96 ± 0.46^{c)} | QSYDYDHY | SGSESNIGANYVT |
| L-CDR3 + 1-opt. | -8-6-47 | 1.97 ± 0.29 | 0.62 ± 0.04 | 3.18 ± 0.33^{c)} | QSYDYDHY | SGSESNIGSNYVS |
| L-CDR3 + 1-opt. | -8-10-57 | 1.65 ± 0.21 | 0.44 ± 0.06 | 2.67 ± 0.25^{c)} | QSYDLIRH | SGSESNIGNNYVQ |
| L-CDR3 + 1-opt. | -8-27-7 | 1.74 ± 0.21 | 0.57 ± 0.07 | 3.30 ± 0.34^{d)} | QSYDMNVH | SGSESNIGNNYVG |
| L-CDR3 + 1-opt. | -8-27-10 | 1.76 ± 0.21 | 0.53 ± 0.05 | 3.01 ± 0.21^{c)} | QSYDMNVH | SGSESNIGANYVN |
| L-CDR3 + 1-opt. | -8-27-41 | 1.67 ± 0.16 | 0.49 ± 0.03 | 2.93 ± 0.27^{d)} | QSYDMNVH | SGSESNIGNNYVQ |

^{a)}Affinity data of MS-GPC-8 are based on 8 different Fab-preparations which were measured on 4 different chips (2 × 500, 1000, 4000RU)
^{b)}For MS-GPC-8-6 mean and standard deviation of 3 different preparations on 3 different chips (500, 4000, 3000RU) is shown.
^{c)}3000RU MHCII were immobilized on a CM5-chip. For each measurement 7 different concentrations from 1 μM to 16 nM were injected on the surface.
Dissociation time: 150 sec, regeneration was reached by 6 μl 10 mM Glycine pH 2.3 followed by 8 μl 7.5 mM NaOH. For MS-GPC-8-6-19 mean and standard deviation of 4 different preparations are shown whereas for all other binders mean and standard devIation of 3 different preparations are shown.
^{d)}One protein preparation is measured on 3 different chips (3000, 2800 and 6500RU).
^{e)}Affinity determination of maturated MHCII binder on a 4000RU density chips; single measurement.
Molecular weights were determined after size exclusion chromatography and found 100% monomeric with the right molecular weight between 45 and 48 kDa.

TABLE 3a
Affinities of selected lgG4 monoclonal antibodies constructed from F_{ab}'s.
Errors represent standard deviations

| Binder (lgG₄) | kₒₙ [M⁻¹s⁻¹] × 10⁵ | k_{off} [s⁻¹] × 10⁻⁵ | K_{D} [nM] |
|---|---|---|---|
| MS-GPC-8-27-41 | 1.1 ± 0.2 | 3.1 ± 0.4 | 0.31 ± 0.06 |
| MS-GPC-8-6-13 | 0.7 ± 0.1 | 3 ± 1 | 0.5 ± 0.2 |
| MS-GPC-8-10-57 | 0.7 ± 0.2 | 4 ± 1 | 0.6 ± 0.2 |

TABLE 3b
Affinities of binders obtained out of affinity maturation of CDR1 light
chain optimisation following CDR3 heavy chain optimisation. Errors
represent standard deviations

| Binder (F_{ab}) | kₒₙ [M⁻¹s⁻¹] × 10⁵ | k_{off} [s⁻¹] × 10⁻³ | K_{D} [nM] |
|---|---|---|---|
| MS-GPC-8-6-2 | 1.2 ± 0.1 | 0.94 ± 0.07 | 7.6 ± 0.3 |
| MS-GPc-8-6-19 | 1.1 ± 0.1 | 1.0 ± 0.2 | 9 ± 1 |
| MS-GPC-8-6-27 | 1.8 ± 0.2 | 1.1 ± 0.2 | 6.3 ± 0.6 |
| MS-GPC-8-6-45 | 1.20 ± 0.07 | 1.03 ± 0.04 | 8.6 ± 0.6 |
| MS-GPC-8-6-13 | 1.9 ± 0.3 | 0.55 ± 0.05 | 3.0 ± 0.5 |
| MS-GPC-8-6-47 | 2.0 ± 0.3 | 0.62 ± 0.04 | 3.2 ± 0.3 |
| MS-GPC-8-10-57 | 1.7 ± 0.2 | 0.44 ± 0.06 | 2.7 ± 0.3 |
| MS-GPC-8-27-7 | 1.7 ± 0.2 | 0.57 ± 0.07 | 3.3 ± 0.3 |
| MS-GPC-8-27-10 | 1.8 ± 0.2 | 0.53 ± 0.05 | 3.0 ± 0.2 |
| MS-GPC-8-27-41 | 1.7 ± 0.2 | 0.49 ± 0.03 | 2.9 ± 0.3 |

TABLE 3c
Binders obtained out of affinity maturation of GPC8 by CDR3 light
chain optimisation

| Binder (F_{ab}) | kₒₙ [M⁻¹s⁻¹] × 10⁵ | k_{off} [s⁻¹] × 10⁻³ | K_{D} [nM] |
|---|---|---|---|
| MS-GPC 8-18 | 1.06 | 8.3 | 78.3 |
| MS-GPC 8-9 | 1.85 | 16.6 | 90.1 |
| MS-GPC 8-1 | 1.93 | 20.9 | 108 |
| MS-GPC 8-17 | 1.0 | 5.48 | 54.7 |
| MS-GPC-8-6^{a)} | 1.2 +/− 0.1 | 5.5 +/− 0.7 | 8 +/− 12 |

Chip density 4000RU MHCII
^{a)}For MS-GPC-8-6 mean and standard deviation of 3 different preparations on 3 different chips (500, 4000, 3000RU) is shown.

TABLE 3d
Binders obtained out of HuCAL In scFv form and their converted Fabs

| | scFᵥ | | | F_{ab} | | |
|---|---|---|---|---|---|---|
| | kₒₙ | k_{off} [s⁻¹] × | | kₒₙ [M⁻¹s⁻¹] × | k_{off} [s⁻¹] × | |
| Binder | [M⁻¹s⁻¹] × 10⁵ | 10⁻³ | K_{D} [nM] | 10⁵ | 10⁻³ | K_{D} [nM] |
| MS-GPC 1 | 0.413 | 61 | 1500 | 0.639 | 53 | 820 |
| MS-GPC 3 | 0.445 | 530 | 11800 | | | |
| MS-GPC 4 | 0.55 | 550 | 10000 | | | |
| MS-GPC 6 | 0.435 | 200 | 4600 | 0.135 | 114 | 8470 (1 curve) |
| MS-GPC 7 | 0.312 | 254 | 8140 | 0.783 | 190 | 2410 |
| MS-GPC 8 | 0.114 | 76 | 560 | 0.99 +/− | 29.0 +/− | 346^{a) +/-} |
| | | | | 0.40 | 8.4 | 141 |
| MS-GPC 10 | 0.187 | 180 | 9625 | 0.22 | 63 | 2860 |
| MS-GPC 11 | 0.384 | 100 | 2500 | 0.361 | 65 | 1800 |

Chip density 500RU MHCII
^{a)}Affinity data of MS-GPC-8 are based on 8 different Fab-preparations which were measured on 4 different chips (2 × 500, 1000, 4000RU) and are shown with standard deviation.

TABLE 4
Killing efficiency after 4 hour incubation of cells with cross-linked
anti-HLA-DR antibody fragments, and maximum killing after 24
hour incubation

| Cross-linked | Killing efficiency against | Maximum killing against |
|---|---|---|
| Fab fragment | GRANTA | Priess |
| MS-GPC-1 | + | + |
| MS-GPC-6 | + | + |
| MS-GPC-8 | + | + |
| MS-GPC-10 | + | + |
| MS-GPC-8-6 | ++ | ++ |
| MS-GPC-8-17 | ++ | ++ |
| MS-GPC-8-6-13 | +++ | +++ |
| MS-GPC-8-10-57 | +++ | +++ |
| MS-GPC-8-27-41 | +++ | +++ |

TABLE 5
Killing efficiency of anti-HLA-DR lgG antibodies of human composition compared to murine anti-HLA-DR
antibodies against a panel of lymphoid tumor cell lines.

| | | | HLA-DR | % Killing by mAb | | | | | |
|---|---|---|---|---|---|---|---|---|---|
| | | | expression | murine | | | | | |
| | Cell line | | mean-FL | mAbs | | human mAbs | | | |
| Name | DR type | Type | L243 | L243 | 8D1 | MS-GPC-8 | 8-27-41 | 8-10-57 | 8-6-13 |
| LG-2 | 1.1 | B-lymphoblastoid | 458 | 79 | 85 | 86 | 87 | 88 | 82 |
| Prless | 4.4 | B-lymphoblastoid | 621 | 87 | 83 | 85 | 88 | 93 | 74 |
| ARH-77 | 12 | B-lymphoblastoid | 301 | 88 | 73 | 84 | 85 | 88 | 87 |
| GRANTA-519 | 2.11 | B cell non-Hodgkin | 1465 | 83 | 56 | 76 | 78 | 78 | 73 |
| KARPAS-422 | 2.4 | B cell non-Hodgkin | 186 | 25 | 32 | 51 | 66 | 68 | 71 |
| KARPAS-299 | 1.2 | T cell non-HodgkIn | 919 | 78 | 25 | 81 | 82 | 79 | 76 |
| DOHH-2 | 1.2 | B cell lymphoma | 444 | 29 | 23 | 58 | 59 | 60 | 53 |
| SR-786 | 1.2 | T cell lymphoma | 142 | 3 | 8 | 1 | 53 | 44 | 26 |
| MHH-CALL-4 | 1.2 | B-ALL | 348 | 35 | 41 | 43 | 63 | 46 | 43 |
| MN-60 | 10.13 | B-ALL | 1120 | 46 | 22 | 71 | 69 | 66 | 67 |
| BJAB | 12.13 | Burkitt lymph. | 338 | 53 | 59 | 49 | 71 | 67 | 64 |
| RAJI | 10, 17 | Burkitt lymph. | 617 | 69 | 64 | 81 | 84 | 86 | 83 |
| L-428 | 12 | Hodgkin's lymph. | 244 | 82 | 81 | 82 | 91 | 91 | 92 |
| HDLM-2 | | Hodgkin's lymph. | 326 | 77 | 73 | 89 | 88 | 84 | 90 |
| HD-MY-Z | | Hodgkin's lymph. | 79 | 35 | 39 | 49 | 69 | 57 | 72 |
| KM-H2 | | Hodgkin's lymph. | 619 | 81 | 56 | 75 | 86 | 88 | 87 |
| L1236 | | Hodgkin's lymph. | 41 | 52 | 62 | 44 | 63 | 66 | 66 |
| BONNA-12 | | hairy cell leuk. | 2431 | 92 | 91 | 91 | 92 | 91 | 86 |
| HC-1 | | hairy cell leuk. | 372 | 88 | 89 | 89 | 93 | 86 | 93 |
| NALM-1 | 1.4 | CML | 1078 | 44 | 4 | 83 | 82 | 78 | 65 |
| L-363 | | plasma cell leu. | 49 | 6 | 5 | 26 | 26 | 24 | 19 |
| EOL-1 | | AML (eosinophll) | 536 | 22 | 13 | 36 | 69 | 49 | 53 |
| LP-1 | | multiple myeloma | 315 | 12 | 0 | 61 | 73 | 70 | 73 |
| RPMI-8226 | | multiple myeloma | 19 | 6 | 0 | 14 | 29 | 26 | 19 |
| MHH-PREB-1 | | B cell non-Hodgkin | 175 | 3 | 3 | 2 | 4 | 8 | 11 |
| MHH-CALL-2 | | B cell precursor leu. | + | 5 | 5 | | | | |
| OPM-2 | | multiple myeloma | 3 | 13 | 0 | 8 | 1 | 4 | 5 |
| KASUMI-1 | | AML | 5 | 0 | 0 | 8 | 10 | 10 | 6 |
| HL-60 | | AML | 3 | 18 | 0 | 3 | 15 | 9 | 22 |
| LAMA-84 | | CML | 7 | 7 | 9 | 5 | 11 | 5 | 7 |

% Killing: 100-% viable cells after a 4 h treatment with 200 nM murine or 50 nM human mAb at 37° C.

TABLE 6
EC50 values for certain anti-HLA-DR antibody fragments of the invention
in a cell-killing assay against lymphoid tumor cells. All EC50 refer to
nanomolar concentrations of the bivalent agent (lgG or cross-linked Fab)
such that values for cross-linked Fab and lgG forms can be compared.

| | | Cell | EC50 of cell killing (nM) +/− |
|---|---|---|---|
| Antibody fragment | Form | line tested | SE for bivalent agent |
| MS-GPC-1 | Fab | PRIESS | 54 ± 14 |
| MS-GPC-8 | Fab | PRIESS | 31 ± 9 |
| MS-GPC-10 | Fab | PRIESS | 33 ± 5 |
| MS-GPC-8-17 | Fab | PRIESS | 16 ± 4 |
| MS-GPC-8-6-2 | Fab | PRIESS | 8 ± 2 |
| MS-GPC-8-10-57 | Fab | LG2 | 7.2 |
| MS-GPC-8-27-41 | Fab | LG2 | 7.2 |
| MS-GPC-8-27-41 | Fab | PRIESS | 7.7 |
| MS-GPC-8 | lgG4 | PRIESS | 8.3 |
| MS-GPC-8-27-41 | lgG4 | PRIESS | 1.1 ± 0.1 |
| MS-GPC-8-10-57 | lgG4 | PRIESS | 1.1 ± 0.2 |
| MS-GPC-8-27-41 | lgG4 | LG2 | 1.23 ± 0.2 |
| MS-GPC-8-10-57 | lgG4 | LG2 | 1.0 ± 0.1 |
| 8D1 | mlgG | PRIESS | 33 |
| L243 | mlgG | PRIESS | 47 |

TABLE 7
IC50 values for certain anti-HLA-DR antibody fragments of the invention
in an assay to determine IL-2 secretion after antigen-specific stimulation of
T-Hyb 1 cells. IC50 for the lgG forms (bivalent) are represented as molar
concentrations, while in order to provide easy comparison, IC50s for the
Fab forms (monovalent) are expressed in terms of half the concentration of
the Fab to enable direct comparison to lgG forms.

| | | |
|---|---|---|
| | IC50 | |
| | (lgG/nM) | |
| Anti-HLA-DR | ((Fab)/2/nM) | Maximum |

| antibody fragment | Form | Mean | SE | inhibition (%) |
|---|---|---|---|---|
| MS-GPC-8-10-57 | lgG | 0.31 | 0.01 | 100 |
| MS-GPC-8-27-41 | lgG | 0.28 | 0.07 | 100 |
| MS-GPC-8-6-13 | lgG | 0.42 | 0.06 | 100 |
| MS-GPC-8-6-2 | lgG | 3.6 | 1.1 | 100 |
| MS-GPC-8-6 | lgG | 6.7 | 2.0 | 100 |
| MS-GPC-8 | lgG | 11.0 | 0.8 | 100 |
| MS-GPC-8-6-2 | Fab | 4.7 | 1.9 | 100 |
| MS-GPC-8-6-13 | Fab | 2.1 | 0.8 | 100 |
| MS-GPC-8-6-19 | Fab | 5.3 | 0.2 | 100 |
| MS-GPC-8-10-57 | Fab | 2.9 | 1.0 | 100 |
| MS-GPC-8-6-27 | Fab | 3.0 | 1.2 | 100 |
| MS-GPC-8-6-47 | Fab | 2.6 | 0.6 | 100 |
| MS-GPC-8-27-7 | Fab | 5.9 | 2.2 | 100 |
| MS-GPC-8-27-10 | Fab | 7.3 | 1.9 | 100 |
| MS-GPC-8-27-41 | Fab | 3.6 | 0.7 | 100 |
| MS-GPC-8-6 | Fab | 20 | | 100 |
| MS-GPC-8 | Fab | 110 | | 100 |

References

Ausubel, F. M., Brent, R., Kingston, R. E., Moore, D. D., Seidman, J. G., Smith, J. A. and Struhl, K. (1998) Current protocols in molecular biology. John Wiley & Sons, Inc., New York, U.S.A.

Babbitt B, Allen P M, Matsueda G, Habe E, Unanue E R, (1985), Nature 317:359.

Baxevanis, C. N., Wemet, D., Nagy, Z. A., Maurer, P. H., and Klein, J. (1980). Immunogenetics, 11, 617.

Billing, R., and Chatterjee, S. (1983). Transplant. Proc. 15, 649.

Bird, R. E. et al. Single-chain antigen-binding proteins [published erratum appears in Science April 1989 28;244(4903):409]. Science 242, 423-6 (1988).

Bonagura, V. R., Ma, a., McDowell, J., Lewison, A., King, D. W. and Suciu-Foca, N. (1987). Cell. Immunolo., 108(2), 356.

Brinkmann, U., Reiter, Y., Jung, S., Lee, B. & Pastan, I. (1993). A recombinant immunotoxin containing a disulfide-stabilized Fv fragment. Proc. Natl. Acad. Sci. U.S.A 90, 7538-7542.

Brown J H, Jardetsky T S, Gorga J C, Stern U, Urban R G, Strominger J L, Wiley D C., (1993), Nature 364: 33.

Buhmann R, Nolte A, Westhaus D, Emmerich B, Hallek M., (1999) Blood 93: 1992 Current Protocols in Immunology, Vol. 2, 7.21.

Drenou B, Blancheteau V, Burgess D H, Fauchet R, Charron D J, Mooney N A., (1999), J. Immunol. 163: 4115.

Falcioni et al. (1999). Nat Biotechnol. 17: 562-567.

Glockshuber, R., Malia, M., Pfitzinger, I. & Plückthun, A. (1990). A comparison of strategies to stabilize immunoglobulin Fv-fragments. Biochemistry 29, 1362-1367.

Gorga J. C., Foran, J., Burakoff, S. J., Strominger, J. L., (1984) Meth Emzym., 108, 607-613.

Gorga, J. C., Horejsi, V., Johnson, D. R., Raghupathy, R., Strominger, J. L., J. Biol. Chem. 262 (1987)16087-94.

Gorga, J. C., Knudsen, P. J., Foran, J. A., Strominger, J. L., Burakoff, S. J., (1986), Cell. Immunol. 103 160-73.

Heiskanen T, Lundkvist A, Soliymani R, Koivunen E, Vaheri A, Lankinen H (1999) Virology, 262(2), 321.

Hopp, T. P., Prickett, K. S., Price, V. L., Libby, R. T., March, C. J., Cerretti, D. P., Urdal, D. L. & Conlon, P. J. (1988), Bio/Technology 6, 1204-1210.

Huston, J. S. et al. Protein engineering of antibody binding sites: recovery of specific activity in an anti-digoxin single-chain Fv analogue produced in Escherichia coli. Proc Natl Acad Sci USA 85, 5879-83 (1988).

Ito K, Bian H. -J, Molina M, Han J, Magram J, Saar E, Belunis C, Bolin D R, Arceo R, Campbell R, Falcioni F, Vidovic' D, Nagy Z A., (1996), J. Exp. Med. 183: 2635-2644.

Jones et al., (1986), Nature 321: 522-525.

Jonker, M., Schellekens, P. T., Harpprecht, J., and Slingerland, W. (1991), Transplant. Proc., 23, 264.

Jonker, M., van Lambalgen, R., Mitchell, D. J., Durham, S. K., and Steinman, L. (1988), Autoimmunity, 1, 399.

Kahoury E. L. and Marshall L. A., (1990) Cell. Tissue Res., 262(2):217-24

Kashmiri S. V., Iwahashi, M., Tamura., Padlan, E. A., Milenic, D. E. & Sclom, J (2001) Crit Rev Oncol Hematol. 38: 3-16.

Klohe E P, Watts R, Bahl M, Alber C, Yu W -Y, Anderson R, Silver J, Gregersen P K, Karr R K., (1988), J. Immunol. 141: 2158-2164.

Knappik, A. & Plückthun, A., (1994), Biotechniques 17, 754-761.

Knappik, A., Ge, L., Honegger, A., Pack, P., Fischer, M., Wellnhofer, G., Hoess A., Wölle, J., Plückthun, A. and Virnekäs, B., (2000), J. Mol. Biol. 296, 55.

Kozak, M. (1987) J. Mol. Biol. 196, 947.

Kuby, J. Immunology:1994, 2^{nd} edition.

McMichael, S. J., Sasazuki, T., McDevitt, H. O., and Payne, R. O., (1977), Arthritis Rheum., 20, 1037.

Muller et al., (1990), J. Immunol., 145: 4006.

Nagy, Z & Vidovic, D. (1996) WO9617874.

Naquet, P., Marchetto, S., and Pierres, M., (1983), Immunogenetics, 18, 559.

Nepom, G. T., Benacerraf, B., and Germain, R. N., (1981), J. Immunol., 127, 31.

Nepom, G. T., Byers, P., Seyfried, C., Healey, L. A., Wilske, K. R., Stage, D., and Nepom, B. S., (1989), Arthritis Rheum., 32,15.

Ohta, N., Nishimura, Y. K., Tanimoto, K., Horiuchi, Y., Abe, C., Shiokawa, Y., Abe, T., Katigari, M., Yoshiki, T., and Sasazuki, T., (1982), Hum. Immunol., 5,123.

Otten et al (1997) pp 5.4.1-5.4.19 in Current Protocols in Immunology, Eds. Coligan et al. Green & Wiley, New York.

Pack, P. and Plückthun, A., (1992), Biochemistry 31, 1579-1584.

Pack, P., (1994), Ph.D. thesis, Ludwig-Maximilians-Universität München.

Pack, P., Kujau, M., Schroeckh, V., Knüpfer, U., Wenderoth, R., Riesenberg D. and Plückthun, A. (1993), Bio/Technology 11, 1271-1277.

Pichia, S. L., Murali, R. & Bumett, R. M (1997) J Struct Biol. 119:6-16.

Presta, (1992), Curr. Op. Struct. Biol. 2: 593-596.

Riechmann et al., (1988), Nature 332: 323-329.

Rheinnecker, M., Hardt, C., Ilag, L. L., Kufer, P., Gruber, R., Hoess, A., Lupas, A., Rottenberger, C., Plückthun, A. and Pack, P., (1996), J. Immunol. 157, 2989.

Rosenbaum, J. T., Adelman, N. E., and McDevitt, H. O., (1983), J. Exp. Med., 154, 1694.

Sambrook et al., 1989, Molecular Cloning: a Laboratory Manual, 2nd ed.

Schiff, B., Mizrachi, Y., Orgad, S., Yaron, M., and Gazit, I., (1982), Ann. Rheum. Dis., 41, 403.

Schmidt, T. G. M. & Skerra, A. (1993). Prot. Engineering 6, 109-122.

Schmidt, T. G. M. & Skerra, A. (1994). J. Chromatogr. A 676, 337-345.

Schmidt, T. G. M. et al. (1996). J. Mol. Biol. 255, 753-766.

Skerra, A. and Plückthun, A. (1988). Science 240, 1038.

Slavin-Chiorini, D. C., Kashmiri, S. V., Milenic, D. E., Poole, D. J., Bernono, E., Schlom, J. & Hand, P. H (1997) Cancer Biother Radiopharm 12: 305-316.

Smith, R. M., Morgan, A., and Wraith, D. C. (1994). Immunology, 83, 1.

Stasny, P. (1978). N. Engl. J. Med., 298, 869.

Stausbøl-Grøn, B., Wind, T., Kjaer, S., Kahns, L., Hansen, N. J. V., Kristensen, P. and Clark, B. F. C. (1996) FEBS Left. 391, 71.

Stern, A. S: and Podlaski, F. J, (1993) Techniques in Protein Chemistry IV, Academic Press Inc., San Diego, Calif.

Stevens, H. P., Roche, N., Hovius, S. E., and Jonker, M., (1990), Transplant. Proc., 22, 1783.

Tiwari, J., and Terasaki, P., (1985), HLA and disease association. (New York: Springer Verlag).

Vidovic D, Falcioni F, Bolin D R, Nagy Z A., (1995a), Eur. J. Immunol., 25:1326.

Vidovic D, Falcioni F, Siklodi B, Belunis C J, Bolin D R, Ito K, Nagy Z A., (1995b), Eur J. Immunol., 25:3349.

Vidovic D, & Toral, J. (1998) Cancer Letters 128: 127-135.

Virnekäs, B., Ge, L., Plukthun, A., Schneider, K. C., Wellenhofer, G. & Moroney, S. E. (1994) Nucleic Acids Res 22: 5600-5607.

Vose, J. M., Colcher, D., Gobar, L., Bierman, P. J., Augustine, S., Tempero, M., Leichner, P., Lynch, J. C., Goldenberg, D. & Armitage, J. O. (2000) Leuk Lymphoma 38: 91-101.

Voss, S. & Skerra, A. (1997). Protein Eng. 10, 975-982.

Waldor, M. K., Sriram, S., McDevitt, H. O., and Steinman, L. (1983). Proc. Natl. Acad. Sci. USA, 80, 2713.

Winter, G., Griffiths, A. D., Hawkins, R. E. and Hoogenboom, H. R. (1994) Making antibodies by phage display technology. Annu. Rev. Immunol. 12, 433.

Woods et al., (1994), J Exp Med. 180: 173-81.

1. A composition including a polypeptide comprising at least one antibody-based antigen-binding domain of human composition with a binding specificity for an antigen expressed on the surface of a cell, wherein treating cells-expressing said antigen with one or more of said polypeptides causes or leads to suppression of an immune response, and wherein the IC50 for said suppression of an immune response is 1 μM or lower.

2. A composition including a polypeptide comprising at least one antibody-based antigen-binding domain with a binding specificity for human HLA DR antigen, wherein treating cells expressing HLA DR with said polypeptide causes or leads to suppression of an immune response, and wherein said antibody based antigen-binding domain includes a combination of a VH domain and a VL domain, wherein said combination is found in one of the clones taken from the list of MS-GPC-1, MS-GPC-2, MS-GPC-3, MS-GPC-4, MS-GPC-5, MS-GPC-6, MS-GPC-7, MS-GPC-8, MS-GPC-10, MS-GPC-11, MS-GPC-14, MS-GPC-15, MS-GPC-16, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

3. The composition of claim 1, wherein said antigen expressed on the surface of said cell is a human MHC class II antigen.

4. A composition including a polypeptide comprising at least one antibody-based antigen-binding domain with a binding specificity for a human MHC class II antigen with a K_{d} of 1 μM or less, wherein treating cells expressing said antigen with said polypeptide causes or leads to suppression of an immune response.

5. A composition including a polypeptide comprising at least one antibody-based antigen-binding domain with a binding specificity for a human MHC class II antigen with a K_{d} of 1 μM or less, said antibody based antigen-binding domain being isolated by a method which includes isolation of VL and VH domains of human composition from a recombinant antibody library by ability to bind to human MHC class II antigen, wherein treating cells expressing MHC Class II with said polypeptide causes or leads to suppression of an immune response.

6. The composition of claim 5, wherein the method for isolating the antibody based antigen-binding domain includes the further steps of:
a. generating a library of variants at least on of the CDR1, CDR2 and CDR3 s quences of one or both of the VL and VH domains, and
b. isolation of VL and VH domains from the library of variants by ability to bind to human MHC class II antigen with a K_{d} of 1 μM or less.

7. The composition of any of claims 3 to 6, wherein said antibody based antigen-binding domain binds to HLA-DR.

8. The composition of any of claims 2 to 7 wherein said antibody based antigen-binding domain binds to the β-chain of HLA-DR.

9. The composition of claim 8, wherein said antibody based antigen-binding domain binds to an epitope of the first domain of the β-chain of HLA-DR.

10. The composition of any of claims 1 to 9, wherein said cells are lymphoids cells.

11. The composition of any of claims 1 to 9, wherein said cells are non-lymphoid cells and express MHC class II antigens.

12. The composition of any of claims 1 to 11, having an IC₅₀ for suppressing an immune response of 1 μM or less.

13. The composition of any of claims 1 to 11, having an IC50 of inhibition of IL-2 secretion of 1 μM or less.

14. The composition of any of claims 1 to 11, having an IC50 of inhibition of T cell proliferation of 1 μM or less.

15. The composition of any of claims 1 to 14, wherein said antibody based antigen-binding domain binds to one or more HLA-DR types selected from the group consisting of DR1-0101, DR2-15021, DR3-0301, DR4Dw4-0401, DR4Dw10-0402, DR4Dw14-0404, DR6-1302, DR6-1401, DR8-8031, DR9-9012, DRw53-B4*0101 and DRw52-B3*0101.

16. The composition of claim 15, wherein said antibody based antigen-binding domain binds to at least 3 different of said HLA-DR types, preferably to at least 5 different of said HLA-DR types, and more preferably to at least 7 different of said HLA-DR types.

17. The composition of any of claims 3 to 16, wherein said antibody based antigen-binding domain includes a combination of a VH domain and a VL domain, wherein said combination is found in one of the clones taken from the list MS-GPC-1, MS-GPC-2, MS-GPC-3, MS-GPC-4, MS-GPC-5, MS-GPC-6, MS-GPC-7, MS-GPC-8, MS-GPC-10, MS-GPC-11, MS-GPC-14, MS-GPC-15, MS-GPC-16, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

18. The composition of any one of claims 3 to 16, wherein said antibody based antigen-binding domain includes of a combination of HuCAL VH2 and HuCAL Vλ1, wherein the VH CDR3, VL CDR1 and VL CDR3 is found in one of the clones taken from the list of list MS-GPC-1, MS-GPC-4, MS-GPC-7, MS-GPC-8, MS-GPC-10, MS-GPC-11, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.

19. The composition of any of claims 3 to 16, wherein said antibody based antigen-binding domain includes a combination of HUCAL VH2 and HuCAL Vλ1, wherein the VH CDR3 sequence is taken from the consensus CDR3 sequence
nnnnRGnFDn
wherein each n independently represents any amino acid residue; and/or wherein the VL CDR3 sequence is taken from the consensus CDR3 sequence
QSYDnnnn
wherein each n independently represents any amino acid residue.

20. The composition of claim 19, wherein the VH CDR3 sequence is SPRYGAFDY and/or the VL CDR3 sequence is QSYDLIRH or QSYDMNVH.

21. The composition of any of claims 3 to 16, wherein said antibody based antigen-binding domain competes for antigen-binding with an antibody including a combination of HuCAL VH2 and HuCAL Vλ1, wherein the VH CDR3 sequence is taken from the consensus CDR3 sequence
nnnnRGnFDn
each n independently represents any amino acid residue; and/or the VL CDR3 sequence is taken from the consensus CDR3 sequence
QSYDnnnn
each n independently represents any amino acid residue.

22. The composition of claim 21, wherein the VH CDR3 sequence is SPRYGAFDY and/or the VL CDR3 sequence is QSYDLIRH or QSYDMNVH.

23. The composition of any of claims 3 to 22, wherein said antibody based antigen-binding domain includes a VL CDR1 sequence represented in the general formula
SGSnnNIGnNYVn
wherein each n independently represents any amino acid residue.

24. The composition of claim 23, wherein the CDR1 sequence is SGSESNIGNNYVQ.

25. The composition of any one of claims 1 to 24, wherein said suppression of an immune response is brought about by or manifests itself in down-regulation of expression of said antigen expressed on the surface of said cell.

26. The composition of any one of claims 1 to 24, wherein said suppression of an immune response is brought about by or manifests itself in inhibition of the interaction between said cell and other cells, wherein said interaction would normally lead to an immune response.

27. The composition of any one of claims 1 to 24, wherein said suppression of an immune response is brought about by or manifests itself in the killing of said cells.

28. The composition of claim 27, wherein said killing is mediated by treating said cells expressing antigen with a plurality of antibody based antigen-binding domains, wherein said antibody based antigen-binding domains are part of at least one multivalent polypeptide, and where neither cytotoxic entities nor immunological mechanisms are needed to causes or leads to said killing.

29. The composition of claim 27 or 28, wherein said killing affects at least 50%, preferably at least 75%, more preferably at least 85% of activated cells compared to killing of less than 30%, preferably less than 20%, more preferably less than 10% of non activated cells.

30. The composition of claim 27 to 29, wherein said killing is mediated by an innate, pre-programmed process of said cells.

31. The composition of claim 30, wherein said killing is non-apoptotic.

32. The composition of claim 30, wherein said killing is dependent (only?) on the action of non-caspase proteases.

33. The composition of claim 30, wherein said killing is independent of caspases that can be inhibited by zVAD-fmk or zDEVD-fmk.

34. The composition of any one of claims 1 to 33, wherein said composition comprises antibody fragments selected from Fv, scFv, dsFv and Fab fragments.

35. The composition of any one of claims 1 to 33, wherein said composition comprises a F(ab′)2 antibody fragment or a mini-antibody fragment.

36. The composition of any one of claims 1 to 33, wherein said composition comprises at least one full antibody selected from the antibodies of classes IgG_{1, 2}a, 2b, 3, 4, IgA, and IgM.

37. The composition of any one of claims 34 to 36, wherein said composition further comprises a cross-linking moiety or moieties.

38. The composition of claim 37, wherein the antigen-binding sites are cross-linked to a polymer.

39. The composition of any one of claims 1 to 38, formulated in a pharmaceutically acceptable carrier and/or diluent.

40. A pharmaceutical preparation comprising the composition of claim 12 in an amount sufficient to suppress an immune response in an animal, such as where said animal is human.

41. A pharmaceutical preparation comprising the composition of claim 13 in an amount sufficient to inhibit IL-2 secretion in an animal, such as where said animal is human.

42. A pharmaceutical preparation comprising the composition of claim 14 in an amount sufficient to inhibit T cell proliferation in an animal, such as where said animal is human.

43. A diagnostic composition including the composition of any of claims 1 to 38.

44. The use of a composition of any one of claims 1 to 38, for preparing a pharmaceutical preparation for the treatment of animals, such as where said animals are human.

45. A nucleic acid including a protein (need to check definition) coding sequence for a polypeptide of the composition of any of claims 1 to 38.

46. A vector comprising the nucleic acid of claim 45, and a transcriptional regulatory sequence operably linked thereto.

47. A host cell harboring a nucleic acid of claim 45 or the vector of claim 46.

48. A method for the production of an immunosuppressive composition, comprising culturing the cells of claim 47 under conditions wherein the nucleic acid is expressed.

49. A method for suppressing activation of a cell of the immune system, comprising treating the cell with a composition of any of claims 1 to 39.

50. A method for suppressing proliferation of a cell of the immune system, comprising treating the cell with a composition of any of claims 1 to 39.

51. A method for suppressing IL-2 secretion by a cell of the immune system, comprising treating the cell with a composition of any of claims 1 to 39.

52. A method of suppressing the interaction of a cell of the immune system with another cell, comprising contacting the cell with the composition of any of claims 1 to 39.

53. A method for immunosuppressing a patient, comprising administering to the patient an effective amount of a composition of any of claims 1 to 39.

54. A method for killing a cell expressing an antigen on the surface of said cell comprising the step of treating the cell with a composition including a plurality of antibody based antigen-binding domains of any one of claims 1 to 39, wherein said antibody based antigen-binding domains are part of at least one multivalent polypeptide, and where neither cytotoxic entities nor immunological mechanisms are needed to cause or lead to said killing.

55. The method according to claim 54, wherein said antigen is HLA DR.

56. The use according to claim 44, wherein said treatment is the treatment of a disorder selected from rheumatoid arthritis, juvenile arthritis, multiple sclerosis, Grave's disease, insulin-dependent diabetes, narcolepsy, psoriasis, systemic lupus erythematosus, ankylosing spondylitis, transplant rejection, graft vs. host disease, Hashimoto's disease, myasthenia gravis, pemphigus vulgaris, glomerulonephritis, thyroiditis, pancreatitis, insulitis, primary biliary cirrhosis, irritable bowel disease and Sjogren syndrome.

57. The use according to claim 44, wherein said treatment is the treatment of a disorder selected from myasthenia gravis, rheumatoid arthritis, multiple sclerosis, transplant rejection and graft vs. host disease.

58. A method to identify patients that can be treated with a composition of any one of claims 1 to 38, formulated in a pharmaceutically acceptable carrier and/or diluent, comprising the steps of
a. Isolating cells from a patient;
b. Contacting said cells with the composition of any one of claims 1 to 39
c. Measuring the degree of killing, immunosuppression, IL2 secretion or proliferation of said cells.

59. A kit to identify patients that can be treated with a composition of any of claims 1 to 38, formulated in a pharmaceutically acceptable carrier and/or diluent, comprising
d. A composition of any of claims 1 to 39
e. Means to measure the degree of killing or immunosuppression, IL2 secretion or proliferation of said cells.

60. A kit comprising
f. a composition according to any one of claims 1 to 39, and
g. a cross-linking moiety.

61. A kit comprising
h. a composition according to any one of claims 1 to 39 or the diagnostic composition of claim 43, and
i. a detectable moiety or moieties, and
j. reagents and/or solutions to effect and/or detect binding of (i.) to an antigen.

62. A cytotoxic composition comprising a composition of any one of claims 1 to 38 operably linked to a cytotoxic agent.

63. An immunogenic composition comprising a composition of any one of claims 1 to 38 operably linked to an immunogenic agent.

64. A method to kill a cell expressing an antigen on the surface of said cell, comprising contacting said cell with a composition of any one of claims 1 to 38 operably linked to a cytotoxic or immunogenic agent.

65. The use of a composition of any one of claims 1 to 38 operably linked to a cytotoxic or immunogenic agent for the preparation of a pharmaceutical composition for the treatment of animals.

66. A method for conducting a pharmaceutical business comprising:
(i) isolating one or more antibody based antigen-binding domains that bind to MHC class II expressed on the surface of human cells with a Kd of 1 μM or less;
(ii) generating a composition comprising said antibody based antigen-binding domains, which composition is immunosuppressant with an IC50 of 100 nM or less;
(iii) conducting therapeutic profiling of said composition for efficacy and toxicity in animals;
(iv) preparing a package insert describing the use of said composition for immunosuppression therapy; and
(v) marketing said composition for use as an immunosuppressant.

67. A method for conducting a life science business comprising:
(i) isolating one or more antibody based antigen-binding domains that bind to MHC class II expressed on the surface of human cells with a Kd of 1 μM or less;
(ii) generating a composition comprising said antibody based antigen-binding domains, which composition is immunosuppressant with an IC50 of 100 nM or less;
(iii) licensing, jointly developing or selling, to a third party, the rights for selling said composition.

68. The method of any of claims 66 or 67, wherein the antibody based antigen-binding domain is isolated by a method which includes
a. isolation of VL and VH domains of human composition from a recombinant antibody library by ability to bind to HLA DR,
b. generating a library of variants at least one of the CDR1, CDR2 and CDR3 sequences of one or both of the VL and VH domains, and
c. isolation of VL and VH domains from the library of variants by ability to bind to HLA DR with a Kd of 1 μM or less.

69. The method of any of claims 66 to 68, wherein antibody based antigen-binding domain is a combination of VH and VL domains found in the clones taken from the list of MS-GPC-1, MS-GPC-2, MS-GPC-3, MS-GPC-4, MS-GPC-5, MS-GPC-6, MS-GPC-7, MS-GPC-8, MS-GPC-10, MS-GPC-11, MS-GPC-14, MS-GPC-15, MS-GPC-16, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10 and MS-GPC-8-27-41.
